SEQUENCE LISTING (15 sequences)

SEQ ID NO: 1 - DNA, 3399 bp, human
atggaaagatttcgtctggagaagaagttacctggtcctgatgaagaagctgttgtggatcttggcaaaactagctcaactgtgaacaccaagtttgaaaaagaagaactagaaagtcatagagctgtatatattggtgttcacgtcccgtttagtaaagagagtcgtcggcgtcataggcatcgcggacacaaacatcaccaccggagaagaaaagataaagaatcagataaagaagatggacgggaatctccttcttatgatacaccatcccagagagttcagtttatccttggtactgaagatgatgatgaagaacatattccccatgatctcttcacggaaatggatgaactgtgttacagagatggagaagaatatgaatggaaagaaactgctagatggctgaaatttgaagaggatgttgaagatggcggtgaccgatggagtaaaccttatgtggcaactctctctttgcacagtctttttgaactaaggagttgcatcctcaatggaacagtcatgctggatatgagagcaagcactctagatgaaatagcagatatggtattagacaacatgatagcttctggccaattagacgagtccatacgagagaatgtcagagaagctcttctgaagagacatcatcatcagaatgagaaaagattcaccagtcggattcctcttgttcgatcttttgcagatataggcaagaaacattctgaccctcacttgcttgaaaggaatggtattttggcctctccccagtctgctcctggaaacttggacaatagtaaaagtggagaaattaaaggtaatggaagtggtggaagcagagaaaatagtactgttgacttcagcaaggttgatatgaatttcatgagaaaaattcctacgggtgctgaggcatccaacgtcctggtgggcgaagtagactttttggaaaggccaataattgcatttgtgagactggctcctgctgtcctccttacagggttgactgaggtccctgttccaaccaggtttttgtttttgttattgggtccagcgggcaaggcaccacagtaccatgaaattggacgatcaatagccactctcatgacagatgagattttccatgatgtagcttataaagcaaaagacagaaatgacctcttatctggaattgatgaatttttagatcaagtaactgtcctacctccaggagagtgggatccttctatacgcatagaaccaccaaaaagtgtcccttctcaggaaaagagaaagattcctgtgtttcacaatggatctacccccacactgggtgagactcctaaagaggccgctcatcatgctgggcctgagctacagaggactggacggctttttggtggtttgatacttgacatcaaaaggaaagcaccttttttcttgagtgacttcaaggatgcattaagcctgcagtgcctggcctcgattcttttcctatactgtgcctgtatgtctcctgtaatcacttttggagggctgcttggagaagctacagaaggcagaataagtgcaatagagtctctttttggagcatcattaactgggattgcctattcattgtttgctgggcaacctctaacaatattggggagcacaggtccagttctagtgtttgaaaaaattttatataaattctgcagagattatcaactttcttatctgtctttaagaaccagtattggtctgtggacttcttttttgtgcattgttttggttgcaacagatgcaagcagccttgtgtgttatattactcgatttacagaagaggcttttgcagcccttatttgcatcatattcatctacgaggctttggagaagctctttgatttaggagaaacatatgcatttaatatgcacaacaacttagataaactgaccagctactcatgtgtatgtactgaacctccaaaccccagcaatgaaactctagcacaatggaagaaagataatataacagcacacaatatttcctggagaaatcttactgtttctgaatgtaaaaaacttcgtggtgtattcttggggtcagcttgtggtcatcatggaccttatattccagatgtgctcttttggtgtgtcatcttgtttttcacaacattttttctgtcttcattcctcaagcaatttaagaccaagcgttactttcctaccaaggtgcgatcgacaatcagtgattttgctgtatttctcacaatagtaataatggttacaattgactaccttgtaggagttccatctcctaaacttcatgttcctgaaaaatttgagcctactcatccagagagagggtggatcataagcccactgggagataatccttggtggaccttattaatagctgctattcctgctttgctttgtaccattctcatctttatggatcaacaaatcacagctgtaattataaacagaaaggaacacaaattgaagaaaggagctggctatcaccttgatttgctcatggttggcgttatgttgggagtttgctctgtcatgggacttccatggtttgtggctgcaacagtgttgtcaataagtcatgtcaacagcttaaaagttgaatctgaatgttctgctccaggggaacaacccaagtttttgggaattcgtgaacagcgggttacagggctaatgatttttattctaatgggcctctctgtgttcatgacttcagtcctaaagtttattccaatgcctgttctgtatggtgttttcctttatatgggagtttcctcattaaaaggaatccagttatttgaccgtataaaattatttggaatgcctgctaagcatcagcctgatttgatatacctccgttatgtgccgctctggaaggtccatattttcacagtcattcagcttacttgtttggtccttttatgggtgataaaagtttcagctgctgcagtggtttttcccatgatggttcttgcattagtgtttgtgcgcaaactcatggacctgtgtttcacgaagagagaacttagttggcttgatgatcttatgccagaaagtaagaaaaagaaagaagatgacaaaaagaaaaaagagaaagaggaagctgaacggatgcttcaagatgatgatgatactgtgcaccttccatttgaagggggaagtctcttgcaaattccagtcaaggccctaaaatatagtcctgataaacctgtgagtgtgaaaataagttttgaagatgaaccaagaaagaaatacgtggatgctgaaacttcattatagaattgaaccaagaggcattatacatatagatatatacatatgtaatgtgtgcgtatcatgtcactatatataagaatattgtatgtcatgctgtttatgtgtgactaccgggtttttaaaagtagt

SEQ ID NO: 2 - DNA, 3600 bp, human
atggaggctgatggggccggcgagcagatgagaccgctactcacccggggtcctgatgaagaagctgttgtggatcttggcaaaactagctcaactgtgaacaccaagtttgaaaaagaagaactagaaagtcatagagctgtatatattggtgttcacgtcccgtttagtaaagagagtcgtcggcgtcataggcatcgcggacacaaacatcaccaccggagaagaaaagataaagaatcagataaagaagatggacgggaatctccttcttatgatacaccatcccagagagttcagtttatccttggtactgaagatgatgatgaagaacatattccccatgatctcttcacggaaatggatgaactgtgttacagagatggagaagaatatgaatggaaagaaactgctagatggctgaaatttgaagaggatgttgaagatggcggtgaccgatggagtaaaccttatgtggcaactctctctttgcacagtctttttgaactaaggagttgcatcctcaatggaacagtcatgctggatatgagagcaagcactctagatgaaatagcagatatggtattagacaacatgatagcttctggccaattagacgagtccatacgagagaatgtcagagaagctcttctgaagagacatcatcatcagaatgagaaaagattcaccagtcggattcctcttgttcgatcttttgcagatataggcaagaaacattctgaccctcacttgcttgaaaggaatggtattttggcctctccccagtctgctcctggaaacttggacaatagtaaaagtggagaaattaaaggtaatggaagtggtggaagcagagaaaatagtactgttgacttcagcaaggttgatatgaatttcatgagaaaaattcctacgggtgctgaggcatccaacgtcctggtgggcgaagtagactttttggaaaggccaataattgcatttgtgagactggctcctgctgtcctccttacagggttgactgaggtccctgttccaaccaggtttttgtttttgttattgggtccagcgggcaaggcaccacagtaccatgaaattggacgatcaatagccactctcatgacagatgagattttccatgatgtagcttataaagcaaaagacagaaatgacctcttatctggaattgatgaatttttagatcaagtaactgtcctacctccaggagagtgggatccttctatacgcatagaaccaccaaaaagtgtcccttctcaggaaaagagaaagattcctgtgtttcacaatggatctacccccacactgggtgagactcctaaagaggccgctcatcatgctgggcctgagctacagaggactggacggctttttggtggtttgatacttgacatcaaaaggaaagcaccttttttcttgagtgacttcaaggatgcattaagcctgcagtgcctggcctcgattcttttcctatactgtgcctgtatgtctcctgtaatcacttttggagggctgcttggagaagctacagaaggcagaataagtgcaatagagtctctttttggagcatcattaactgggattgcctattcattgtttgctgggcaacctctaacaatattggggagcacaggtccagttctagtgtttgaaaaaattttatataaattctgcagagattatcaactttcttatctgtctttaagaaccagtattggtctgtggacttcttttttgtgcattgttttggttgcaacagatgcaagcagccttgtgtgttatattactcgatttacagaagaggcttttgcagcccttatttgcatcatattcatctacgaggctttggagaagctctttgatttaggagaaacatatgcatttaatatgcacaacaacttagataaactgaccagctactcatgtgtatgtactgaacctccaaaccccagcaatgaaactctagcacaatggaagaaagataatataacagcacacaatatttcctggagaaatcttactgtttctgaatgtaaaaaacttcgtggtgtattcttggggtcagcttgtggtcatcatggaccttatattccagatgtgctcttttggtgtgtcatcttgtttttcacaacattttttctgtcttcattcctcaagcaatttaagaccaagcgttactttcctaccaaggtgcgatcgacaatcagtgattttgctgtatttctcacaatagtaataatggttacaattgactaccttgtaggagttccatctcctaaacttcatgttcctgaaaaatttgagcctactcatccagagagagggtggatcataagcccactgggagataatccttggtggaccttattaatagctgctattcctgctttgctttgtaccattctcatctttatggatcaacaaatcacagctgtaattataaacagaaaggaacacaaattgaagaaaggagctggctatcaccttgatttgctcatggttggcgttatgttgggagtttgctctgtcatgggacttccatggtttgtggctgcaacagtgttgtcaataagtcatgtcaacagcttaaaagttgaatctgaatgttctgctccaggggaacaacccaagtttttgggaattcgtgaacagcgggttacagggctaatgatttttattctaatgggcctctctgtgttcatgacttcagtcctaaagtttattccaatgcctgttctgtatggtgttttcctttatatgggagtttcctcattaaaaggaatccagttatttgaccgtataaaattatttggaatgcctgctaagcatcagcctgatttgatatacctccgttatgtgccgctctggaaggtccatattttcacagtcattcagcttacttgtttggtccttttatgggtgataaaagtttcagctgctgcagtggtttttcccatgatggttcttgcattagtgtttgtgcgcaaactcatggacctgtgtttcacgaagagagaacttagttggcttgatgatcttatgccagaaagtaagaaaaagaaagaagatgacaaaaagaaaaaagagaaagaggaagctgaacggatgcttcaagatgatgatgatactgtgcaccttccatttgaagggggaagtctcttgcaaattccagtcaaggccctaaaatatagtgttgatccctcaattgttaacatatcagatgaaatggccaaaactgcacagtggaaggcactttccatgaatactgagaatgccaaagtaaccagatctaacatgagtcctgataaacctgtgagtgtgaaaataagttttgaagatgaaccaagaaagaaatacgtggatgctgaaacttcattatagaattgaaccaagaggcattatacatatagatatatacatatgtaatgtgtgcgtatcatgtcactatatataagaatattgtatgtcatgctgtttatgtgtgactaccgggtttttaaaagtagtgtctggagtttgtaatgagcaccgtggagactatgtatttaatgaaatgctctctttgaagtgaggtacatggttctt

SEQ ID NO: 3 - protein, 1090 aa, human
MERFRLEKKLPGPDEEAVVDLGKTSSTVNTKFEKEELESHRAVYIGVHVPFSKESRRRHRHRGHKHHHRRRKDKESDKEDGRESPSYDTPSQRVQFILGTEDDDEEHIPHDLFTEMDELCYRDGEEYEWKETARWLKFEEDVEDGGDRWSKPYVATLSLHSLFELRSCILNGTVMLDMRASTLDEIADMVLDNMIASGQLDESIRENVREALLKRHHHQNEKRFTSRIPLVRSFADIGKKHSDPHLLERNGILASPQSAPGNLDNSKSGEIKGNGSGGSRENSTVDFSKVDMNFMRKIPTGAEASNVLVGEVDFLERPIIAFVRLAPAVLLTGLTEVPVPTRFLFLLLGPAGKAPQYHEIGRSIATLMTDEIFHDVAYKAKDRNDLLSGIDEFLDQVTVLPPGEWDPSIRIEPPKSVPSQEKRKIPVFHNGSTPTLGETPKEAAHHAGPELQRTGRLFGGLILDIKRKAPFFLSDFKDALSLQCLASILFLYCACMSPVITFGGLLGEATEGRISAIESLFGASLTGIAYSLFAGQPLTILGSTGPVLVFEKILYKFCRDYQLSYLSLRTSIGLWTSFLCIVLVATDASSLVCYITRFTEEAFAALICIIFIYEALEKLFDLGETYAFNMHNNLDKLTSYSCVCTEPPNPSNETLAQWKKDNITAHNISWRNLTVSECKKLRGVFLGSACGHHGPYIPDVLFWCVILFFTTFFLSSFLKQFKTKRYFPTKVRSTISDFAVFLTIVIMVTIDYLVGVPSPKLHVPEKFEPTHPERGWIISPLGDNPWWTLLIAAIPALLCTILIFMDQQITAVIINRKEHKLKKGAGYHLDLLMVGVMLGVCSVMGLPWFVAATVLSISHVNSLKVESECSAPGEQPKFLGIREQRVTGLMIFILMGLSVFMTSVLKFIPMPVLYGVFLYMGVSSLKGIQLFDRIKLFGMPAKHQPDLIYLRYVPLWKVHIFTVIQLTCLVLLWVIKVSAAAVVFPMMVLALVFVRKLMDLCFTKRELSWLDDLMPESKKKKEDDKKKKEKEEAERMLQDDDDTVHLPFEGGSLLQIPVKALKYSPDKPVSVKISFEDEPRKKYVDAETSL

SEQ ID NO: 4 - protein, 1131 aa, human
MEADGAGEQMRPLLTRGPDEEAVVDLGKTSSTVNTKFEKEELESHRAVYIGVHVPFSKESRRRHRHRGHKHHHRRRKDKESDKEDGRESPSYDTPSQRVQFILGTEDDDEEHIPHDLFTEMDELCYRDGEEYEWKETARWLKFEEDVEDGGDRWSKPYVATLSLHSLFELRSCILNGTVMLDMRASTLDEIADMVLDNMIASGQLDESIRENVREALLKRHHHQNEKRFTSRIPLVRSFADIGKKHSDPHLLERNGILASPQSAPGNLDNSKSGEIKGNGSGGSRENSTVDFSKVDMNFMRKIPTGAEASNVLVGEVDFLERPIIAFVRLAPAVLLTGLTEVPVPTRFLFLLLGPAGKAPQYHEIGRSIATLMTDEIFHDVAYKAKDRNDLLSGIDEFLDQVTVLPPGEWDPSIRIEPPKSVPSQEKRKIPVFHNGSTPTLGETPKEAAHHAGPELQRTGRLFGGLILDIKRKAPFFLSDFKDALSLQCLASILFLYCACMSPVITFGGLLGEATEGRISAIESLFGASLTGIAYSLFAGQPLTILGSTGPVLVFEKILYKFCRDYQLSYLSLRTSIGLWTSFLCIVLVATDASSLVCYITRFTEEAFAALICIIFIYEALEKLFDLGETYAFNMHNNLDKLTSYSCVCTEPPNPSNETLAQWKKDNITAHNISWRNLTVSECKKLRGVFLGSACGHHGPYIPDVLFWCVILFFTTFFLSSFLKQFKTKRYFPTKVRSTISDFAVFLTIVIMVTIDYLVGVPSPKLHVPEKFEPTHPERGWIISPLGDNPWWTLLIAAIPALLCTILIFMDQQITAVIINRKEHKLKKGAGYHLDLLMVGVMLGVCSVMGLPWFVAATVLSISHVNSLKVESECSAPGEQPKFLGIREQRVTGLMIFILMGLSVFMTSVLKFIPMPVLYGVFLYMGVSSLKGIQLFDRIKLFGMPAKHQPDLIYLRYVPLWKVHIFTVIQLTCLVLLWVIKVSAAAVVFPMMVLALVFVRKLMDLCFTKRELSWLDDLMPESKKKKEDDKKKKEKEEAERMLQDDDDTVHLPFEGGSLLQIPVKALKYSVDPSIVNISDEMAKTAQWKALSMNTENAKVTRSNMSPDKPVSVKISFEDEPRKKYVDAETSL

SEQ ID NO: 5 - protein, 30 aa, human
SPVITFGGLLGEATEGRISAIESLFGASLT

SEQ ID NO: 6 - protein, 78 aa, human
KLFDLGETYAFNMHNNLDKLTSYSCVCTEPPNPSNETLAQWKKDNITAHNISWRNLTVSECKKLRGVFLGSACGHHGP

SEQ ID NO: 7 - protein, 78 aa, mouse
KLFHLGEIYAFNMHNNLDELTSYTCVCAEPSNPSNETLELWKRKNITAYSVSWGNLTVSECKTFHGMFVGSACGPHGP

SEQ ID NO: 8 - protein, 30 aa, human
PSPKLHVPEKFEPTHPERGWIISPLGDNPW

SEQ ID NO: 9 - protein, 30 aa, mouse
PSPKLHVPEKFEPTDPSRGWIISPLGDNPW

SEQ ID NO: 10 - protein, 30 aa, human
SISHVNSLKVESECSAPGEQPKFLGIREQR

SEQ ID NO: 11 - protein, 608 aa, human
MERFRLEKKLPGPDEEAVVDLGKTSSTVNTKFEKEELESHRAVYIGVHVPFSKESRRRHRHRGHKHHHRRRKDKESDKEDGRESPSYDTPSQRVQFILGTEDDDEEHIPHDLFTEMDELCYRDGEEYEWKETARWLKFEEDVEDGGDRWSKPYVATLSLHSLFELRSCILNGTVMLDMRASTLDEIADMVLDNMIASGQLDESIRENVREALLKRHHHQNEKRFTSRIPLVRSFADIGKKHSDPHLLERNGEGLSASRHSLRTGLSASNLSLRGESPLSLLLGHLLPSSRAGTPAGSRCTTPVPTPQNSPPSSPSISRLTSRSSQESQRQAPELLVSPASDDIPTVVIHPPEEDLEAALKGEEQKNEENVDLTPGILASPQSAPGNLDNSKSGEIKGNGSGGSRENSTVDFSKVDMNFMRKIPTGAEASNVLVGEVDFLERPIIAFVRLAPAVLLTGLTEVPVPTRFLFLLLGPAGKAPQYHEIGRSIATLMTDEIFHDVAYKAKDRNDLLSGIDEFLDQVTVLPPGEWDPSIRIEPPKSVPSQEKRKIPVFHNGSTPTLGETPKEAAHHAGPELQRTGRLFGGLILDIKRKAPFFLSDFKDALSLQC

SEQ ID NO: 12 - protein, 36 aa, human
ATVLSISHVNSLKVESECSAPGEQPKFLGIREQRVT

SEQ ID NO: 13 - protein, 37 aa, human
DRIKLFGMPAKHQPDLIYLRYVPLWKVHIFTVIQLTC

SEQ ID NO: 14 - protein, 35 aa, human
HNNLDKLTSYSCVCTEPPNPSNETLAQWKKDNITA

SEQ ID NO: 15 - protein, 35 aa, human
LAQWKKDNITAHNISWRNLTVSECKKLRGVFLGSA

(19) United States
(12) Patent Application Publication
(10) Pub. No.: US 2018/0057588 A1
(43) Pub. Date: Mar. 1, 2018

(54) Antibodies Towards an Extracellular Region of NBCn1

(71) Applicant: Aarhus Universitet, Aarhus (DK)

(72) Inventor: Ebbe Briggs BØDTKJER, Skanderborg (DK)

(21) Appl. No.: 15/558,392

(22) PCT Filed: Mar. 16, 2016
(86) PCT No.: PCT/DK2016/050073

§ 371 Date: Sep. 14, 2017

(30) Foreign Application Priority Data
Mar. 16, 2015 (DK) PA 2015 70148

(51) Int. Cl. C07K 16/28; A61K 39/395; A61K 45/06

(52) U.S. Cl. CPC C07K 16/28; A61K 39/39558; A61K 2039/505; C07K 2317/76; A61K 45/06

(57) ABSTRACT

The present application discloses antibodies or antigen binding fragment thereof specifically recognizing and binding an extracellular polypeptide region of human NBCn1. Experimental data are detailed for mouse NBCn1 antibodies. The medical use of such antibodies is claimed, in particular for the treatment of hyperproliferative disorders, such as cancer, or atherosclerosis and/or restenosis.

### FIELD OF INVENTION

The present invention relates to the use of NBCn1 (SLC4A7) as a target for treatment of breast cancer, atherosclerosis and/or restenosis.

### BACKGROUND OF INVENTION

The lifetime risk of developing breast cancer is more than 1 in 8 for women in the Western world and globally more than 500,000 women die of breast cancer each year. Like most solid cancers, breast carcinomas are characterized by high metabolic rates and prominent glycolytic activities. Metabolic production of CO₂ and lactic acid represent substantial sources of intracellular acid loading that in breast cancer cells are compensated by upregulated cellular net acid extrusion. High rates of net transmembrane acid efflux in breast carcinomas maintain intracellular pH (pHᵢ) equal to or higher than in normal breast tissue. In combination with an insufficient blood supply, the upregulated cellular acid extrusion also creates an acidic and hypoxic extracellular tumor microenvironment. This characteristic compartmentalization of acid-base equivalents has been proposed to promote cancer progression; and as a consequence, cancer metabolism and mechanisms of acidic waste product elimination could be relevant targets for anti-cancer treatment.

The high proliferative activity and growth rate of cancer tissue are energy demanding, and inhibition of glycolytic activity is a possible therapeutic approach. Phosphofructokinase 1 (PFK1) is the rate limiting step of glycolysis and has prominent pH sensitivity in vitro. In vivo, pHᵢ may also regulate glycolytic activity. Thus, interfering with acid-base regulation—to lower pHᵢ selectively in cancer cells—may potentially inhibit glycolytic energy production in cancer tissue without major effects on normal tissue.

Targeting Na⁺/H⁺-exchangers (Loo S Y, Chang M K, Chua C S, Kumar A P, Pervaiz S, Clement M V. NHE-1: a promising target for novel anti-cancer therapeutics. Curr. Pharm. Des 2012;18:1372-1382), monocarboxylate transporters (Chiche J et al. In vivo pH in metabolic-defective Ras-transformed fibroblast tumors: key role of the monocarboxylate transporter, MCT4, for inducing an alkaline intracellular pH. Int. J. Cancer 2012; 130:1511-1520) or H⁺-pumps (De Milito A, Marino M L, Fais S. A rationale for the use of proton pump inhibitors as antineoplastic agents. Curr. Pharm. Des 2012; 18:1395-1406), interfering with acid-base buffering by NaHCO₃ supplementation (Silva A S, Yunes J A, Gillies R J, Gatenby R A. The potential role of systemic buffers in reducing intratumoral extracellular pH and acid-mediated invasion. Cancer Res. 2009; 69:2677-2684) or inhibiting carbonic anhydrases (Robertson N, Potter C, Harris A L. Role of carbonic anhydrase IX in human tumor cell growth, survival, and invasion. Cancer Res. 2004; 64:6160-6165; Swietach P, Patiar S, Supuran C T, Harris A L, Vaughan-Jones R D. The role of carbonic anhydrase 9 in regulating extracellular and intracellular ph in three-dimensional tumor cell growths. J. Biol. Chem. 2009; 284:20299-20310) have been suggested as possible treatment strategies for different types of cancer. For human breast cancer, Na⁺,HCO₃⁻-cotransport is involved in acid extrusion and is also functionally upregulated during breast carcinogenesis (Lee S, Mele M, Vahl P, Christiansen P M, Jensen V E D, Boedtkjer E. Na⁺,HCO₃⁻-cotransport is functionally upregulated during human breast carcinogenesis and required for the inverted pH gradient across the plasma membrane. Pflugers Arch. 2014; Boedtkjer E et al. Contribution of Na⁺,HCO₃⁻-cotransport to cellular pH control in human breast cancer: a role for the breast cancer susceptibility locus NBCn1 (SLC4A7). Int. J. Cancer 2013; 132:1288-1299). However, nothing is known about the consequences of inhibiting Na⁺,HCO₃⁻-cotransport for development and progression of breast cancer or for atherosclerosis or restenosis.

Known and putative Na³⁰-dependent HCO₃⁻-transporters are gathered in the SLC4 family, which comprise 7 members. There are no suitable selective pharmacological inhibitors of Na⁺,HCO₃⁻-cotransporters. Thus, there is a need for identifying selective inhibitors of Na⁺,HCO₃⁻-cotransporters and evaluation of their potential as anticancer agents.

### SUMMARY OF INVENTION

The invention relates to the use of the Na⁺,HCO₃⁻-cotransporter NBCn1 (SLC4A7) as a target for breast cancer, atherosclerosis and/or restenosis treatment. The inventor has found in a mouse breast cancer model that knockout of NBCn1 leads to reduced tumor growth and increased survival. NBCn1 is involved in intracellular pH control and the inventor has found that the intracellular pH in breast tumors of NBCn1 knockout mice is lower compared to wild type mice. These observations support a role of NBCn1 in maintaining a steady state intracellular pH in breast cancer tissue, which thereby allows a high metabolic activity even in hypoxic tumors.

In particular, the invention relates to antibody-based therapy for breast cancer, where the antibodies specifically target extracellular domains of NBCn1. The present disclosure provides novel antibodies with specificity against specific extracellular regions of the NBCn1 transmembrane protein.

### DESCRIPTION OF DRAWINGS

FIG. 1. Disrupting NBCn1 expression delays breast cancer development and tumor growth rate following treatment with MPA and DMBA. A. Breast tumor-free survival of NBCn1 KO and WT mice. Day 0 corresponds to the day of the last DMBA treatment by gavage. The Kaplan-Meier curves were compared by a Gehan-Breslow-Wilcoxon test. B. Representative images of breast tumors from WT and NBCn1 KO mice and average tumor sizes (n=13-16) measured 14 days after the tumors were first detectable. Tumors were typically 3-4 mm at first detection consistent with previous studies. Due to the delayed breast tumor development in the NBCn1 KO mice, they were on average 41 days older than the WT mice on the day of tumor size determination. Log-transformed data for tumor latency and tumor burden were compared between genotypes by unpaired two-tailed Student's t-test. *P<0.05, **P<0.01 vs. WT.

FIG. 2. Histopathology of chemically induced breast cancers differs between WT and NBCn1 KO mice. A. The distribution between histopathological breast cancer subtypes in WT and NBCn1 KO mice (n=13-16). B-F. Hematoxylin and eosin stained histological images representative of the identified breast cancer subtypes: adenosquamous carcinoma (B), squamous carcinoma (C), Wnt type tumor (D), adenomyoepithelioma (E) and adenocarcinoma (F). G. Representative images of hematoxylin and eosin stained (upper panel) and anti-CD45 labeled (lower panel) lymphomas. H. The left panel displays matched values for tumor burden and tumor latency for the individual histopathological breast cancer subtypes. The right panel shows ‘aggressiveness score’ defined as the ratio between tumor burden and tumor latency for each breast tumor subtype. The aggressiveness score was significantly lower (P<0.05, two-way ANOVA) for tumors of NBCn1 KO than WT mice. The scale bar in B represents 200 μm; all images are shown at the same magnification.

FIG. 3. NBCn1 is responsible for net acid extrusion from breast cancer and for the alkaline shift in pHᵢ occurring during breast carcinogenesis. A. Epithelial organoids freshly isolated from breast cancer and matched normal breast tissue. Similar images were obtained from NBCn1 KO and WT mice (not shown). The left panels show cytokeratin 19 (CK-19) immunofluorescence images, center panels show negative control images without primary antibody (no 1° AB), and the right panels show images of BCECF-loaded organoids. B. Average traces of the pH, dynamics during NH₄³⁰ prepulse experiments (n=7). For simplicity, standard errors are shown for a few points only. Recordings were collected at 0.2 Hz. C. Buffering capacities calculated from the change in pH, upon washout of NH₄Cl (n=10-11). D+E. Na⁺-dependent cellular net acid extrusion (or equivalently, base uptake) in epithelial organoids from WT (D) and NBCn1 KO (E) mice as functions of pHᵢ. Experiments were performed on organoids isolated from breast cancer or matched normal breast tissue (n=7-9) in the presence and absence of CO₂/HCO₃⁻. F. Steady-state pHᵢ of epithelial organoids isolated from breast cancer and matched normal breast tissue from NBCn1 KO and WT mice (n=7-9). Comparisons were performed by least-squares linear regression analyses (panel D+E) or by two-tailed unpaired Student's t-test (panel F). *P<0.05 vs. normal breast tissue under similar conditions or as specified.

FIG. 4. Protein expression of NBCn1, MCT1 and MCT4—but not of NHE1—is upregulated during breast carcinogenesis. A-D. Representative immunoblots and average protein expression levels of NBCn1 (A), NHE1 (B), MCT1 (C) and MCT4 (D) in organoids isolated from breast cancer and matched normal breast tissue from NBCn1 KO and WT mice (n=8-10). In the right hand panels, the tumor values are re-plotted as functions of tumor volume. β-actin and dynactin subunit p150^{Glued} were used as loading controls. The representative immunoblots are from the same gels and illustrate samples of matched breast cancer and normal breast tissue. Expression levels are expressed relative to normal breast tissue from WT mice. Log-transformed data were compared by repeated measures two-way ANOVA followed by Bonferroni post-tests (left panels) or by least-squares liner regression analyses (right panels). If the fitted lines were not significantly different between WT and NBCn1 KO mice, only the combined line is shown. *P<0.05, **P<0.01, ***P<0.001, NS: not significantly different vs. organoids of similar source (normal breast or breast cancer) from WT mice or between organoids from normal and cancer tissue as indicated.

FIG. 5. In vivo, glycolytic metabolism is inhibited in breast cancer of NBCn1 KO compared to WT mice. A+B. Interstitial concentrations of glucose (A, n=11-12) and lactate (B, n=12) measured in microdialysis samples obtained with probes placed in breast cancer and matched normal breast tissue of NBCn1 KO and WT mice. C. [lactate]/[glucose] ratios in breast cancer and normal breast tissue (n=11-12; left panel). In the right hand panel, the cancer values are re-plotted as a function of tumor volume (right panel). D+E. Interstitial concentrations of pyruvate (D, n=6-13) and glycerol (E, n=12) measured in microdialysis samples obtained with probes placed in breast cancer and matched normal breast tissue of NBCn1 KO and WT mice. Log-transformed data were compared by repeated measures two-way ANOVA followed by Bonferroni post-tests or by least-squares linear regression analysis (right part of panel C). Because the fitted lines were not significantly different between WT and NBCn1 KO mice, only the combined line is shown. *P<0.05, **P<0.01, NS: not significantly different vs. WT under similar conditions or between organoids from normal and cancer tissue as indicated.

FIG. 6. Protein expression levels and phosphorylation states differ in breast cancer from NBCn1 KO and WT mice. A-E. Representative immunoblots and average protein expression levels of phosphorylated ErbB2 (A), cleaved PARP-1 (B), phosphorylated AMPK (C), phosphorylated Akt (D) and phosphorylated ERK1/2 (E) in organoids isolated from breast cancer and matched normal breast tissue from NBCn1 KO and WT mice (n=8-10). For each protein, the right hand panel shows the tumor values re-plotted as a function of tumor volume. β-actin was used as loading control. The representative immunoblots are from the same gels and illustrate samples of matched breast cancer and normal breast tissue. Expression levels are expressed relative to normal breast tissue from WT mice. Log-transformed data were compared by repeated measures two-way ANOVA followed by Bonferroni post-tests (left panels) or by least-squares linear regression analyses (right panels). Because the fitted lines were not significantly different between WT and NBCn1 KO mice, only the combined lines are shown. *P<0.05, **P<0.01, NS: not significantly different vs. WT under similar conditions or between organoids from normal and cancer tissue as indicated.

FIG. 7. Cell proliferation is reduced in breast cancer from NBCn1 KO compared to WT mice. A+B. Representative immunohistochemical staining for Ki-67 (A) and pHH₃ (B) in sections of breast cancer tissue from NBCn1 KO and WT mice. Examples of positively stained cells are indicated by arrowheads. C+D. Fraction of epithelial cells positive for Ki-67 (C) or pHH₃ (D) in sections of cancer tissue and normal breast tissue from NBCn1 KO and WT mice (n=12-13). From each of the cancer samples, we evaluated 3174±1147 (mean±SD) cells for Ki-67 and 5043±1609 cells for pHH₃ staining. The data were compared by two-way ANOVA followed by Bonferroni post-tests. E. The cancer values from panel C and D were re-plotted as functions of tumor volume. The shaded area highlights the tumors within the interval between the smallest tumor from WT mice and the largest tumor from NBCn1 KO mice. Following normalization to the average cancer value, the matched data for Ki-67 and pHH₃ within the shaded area were compared between WT and NBCn1 KO by repeated measures two-way ANOVA. **P<0.01, ***P<0.001, NS: not significantly different vs. WT under similar conditions, between organoids from normal and cancer tissue, or as indicated. The scale bar in A represents 50 μm; all images are shown at the same magnification.

FIG. 8. NBCn1 mediates the Na⁺,HCO₃⁻-cotransport in VSMCs of mouse carotid arteries and is important for steady-state pH, regulation. A+B. Average pHᵢ traces from NH₄⁺-prepulse experiments showing the response of carotid arteries from NBCn1 KO and WT mice (n=3-5) investigated in the presence (A) or absence (B) of CO₂/HCO₃⁻. SEM is shown for a few points only to improve readability; measurements were performed at 0.2 Hz. C. Rates of pHᵢ recovery in VSMCs of carotid arteries from NBCn1 KO and WT mice after NH₄⁺-prepulses in presence or absence of CO₂/HCO₃⁻(n=3-5). D. Resting steady-state pHᵢ in carotid arteries from NBCn1 KO and WT mice (n=3-9) in presence and absence of CO₂/HCO₃⁻. Data were compared by two-way ANOVA followed by Bonferroni post-tests. *P<0.05, NS: Not significantly different.

FIG. 9. NBCn1 KO inhibits remodeling following carotid artery ligation. A. Representative images of non-ligated, partially ligated, and completely ligated common carotid arteries four weeks after surgery. Arrows indicate the outer boundaries of the lumen (green), media (red) and adventitia (black). The asterisks indicate intraluminal blood clots. The scale bar represents 100 μm, all images are shown at the same magnification. B-D. Relative differences in internal diameter (B), media thickness (C) and media area (D) between ligated and contralateral, non-ligated common carotid arteries. Effects of partial ligation (n=12-13), complete ligation (n=12-14) and sham operation (n=5-6) are shown. For the decrease in lumen diameter, the effect of partial ligation was greater than the effect of complete ligation (P<0.05, two-way ANOVA). The increase in media thickness (P=0.88) and area (P=0.36) did not differ significantly between the complete and partial ligation models (two-way ANOVA). E. Cross-sectional areas of adherent adventitia in ligated common carotid arteries (n=9-13). The amount of adherent adventitia was significantly larger after partial than complete carotid artery ligation (P<0.05, two-way ANOVA) but did not differ between NBCn1 KO and WT mice (P=0.50). Non-ligated common carotid arteries had negligible amounts of adherent adventitia (see panel A). Comparisons were performed by two-way ANOVA and the overall significance level reported. *P<0.05, NS: Not significantly different.

FIG. 10. Na⁺,HCO₃⁻-cotransport via NBCn1 is important for pH, regulation in primary aortic explants. A. Rates of pHᵢ recovery in primary explants investigated in presence of CO₂/HCO₃⁻ and 1 mM amiloride (n=8). Na⁺-dependent pHᵢ recovery rates were calculated at average pHᵢ levels of 6.54±0.12 (WT) and 6.53±0.13 (NBCn1 KO). B. Rates of pHᵢ recovery in primary explants investigated in absence of CO₂/HCO₃⁻ (n=7-12). Na⁺-dependent pHᵢ recovery rates were calculated at average pHᵢ levels of 6.59±0.03 (WT) and 6.59±0.06 (NBCn1 KO). C. Steady-state pHᵢ in primary explants (n=8). D. Rates of pH, recovery in primary explants from WT mice in presence of CO₂/HCO₃⁻ with or without S0859 (n=4-5). Na⁺-dependent pHᵢ recovery rates were calculated at average pHᵢ levels of 6.19±0.08 (control), 6.10±0.02 (5 μM S0859), and 6.10±0.03 (30 μM S0859). E. Effects of S0859 on steady-state pHᵢ (n=6). Curves show mean values obtained five minutes after addition of S0859. Asterisks indicate significant difference vs. WT (panel A and B), vs. control conditions (panel D), vs. baseline and washout conditions (panel E), or as indicated (panel C). Comparisons were performed by paired (panel C) or unpaired (panel A,B) two-tailed Student's t-test, or one-way ANOVA followed by Bonferroni post-tests (panel D,E). *P<0.05, **P<0.01, NS: Not significantly different.

FIG. 11. Inhibition of Na⁺,HCO₃⁻-cotransport attenuates wound healing in scratch assays. A. Representative images showing the extent of wound healing over a 3-hour period in primary aortic explants from WT and NBCn1 KO mice. Red arrows indicate the migrating front. Original series of time-lapse images are included in the online supplement. The scale bar represents 200 μm, all images are shown at the same magnification. B-G. Progression of wound healing after scratch infliction through primary explants (n=5-11). Panels B and D-G report data from aortic explants, whereas panel C reports data from carotid artery explants. Genotype and experimental conditions are given in the individual panels. PSS was used to avoid binding of S0859 to medium and serum components. Comparisons were performed by repeated measures two-way ANOVA. ***P<0.001, NS: Not significantly different.

FIG. 12. Global pHᵢ modulates VSMC proliferation in vitro. A+B. Fraction of cells from primary aortic (A) and carotid (B) explants undergoing mitosis in serum-containing medium or PSS during 6-hour time-lapse recordings (n=5-12). The relative number of cell divisions was significantly lower in the absence (shaded blue) than presence of CO₂/HCO₃⁻ (P<0.01, two-way ANOVA). The image sequence illustrates a cell division. The scale bar represents 20 μm, all images are shown at the same magnification. C. Fraction of BrdU-positive cells in primary explants incubated for six hours with the thymidine-analogue in serum-containing medium (n=7). Representative images are shown. The scale bar represents 200 μm, all images are shown at the same magnification. Red represents anti-BrdU fluorescence; green represents SYTO® 16 nuclear fluorescence. D. Media hypertrophy (i.e., difference in media area between ligated and contralateral, non-ligated carotid arteries; left ordinate) and VSMC proliferation (i.e., fraction of dividing cells in primary aortic explants; right ordinate) as function of global pHᵢ measured in carotid arteries and primary aortic explants, respectively, under the given conditions. Open symbols indicate experiments performed in PSS rather than serum-containing culture medium but otherwise under conditions corresponding to the filled symbols. E. Projected cell areas in primary aortic explants from WT and NBCn1 KO mice (n=16-19) investigated in serum-supplemented medium. F. Images of primary explants from WT and NBCn1 KO mice 18 hours after scratch infliction. Images are representative of 10-11 experiments performed in serum-containing medium with CO₂/HCO₃⁻. The scale bar represents 200 μm, both images are shown at the same magnification. Numbers in individual panels represent the relative number of cells with normal cell morphology after 18 hours of imaging compared to immediately after scratch infliction. G+H. Images of primary explants under control conditions or during exposure to S0859. The scale bars represent 200 μm, all images are shown at same magnification. Experiments were performed in PSS with (G) or without (H) CO₂/HCO3 to avoid binding of S0859 to medium and serum components. The images are representative of 5-10 experiments. Numbers in individual panels represent the relative number of cells with normal cell morphology after 18 hours of imaging compared to immediately after scratch infliction; the gradual decay in proportion of viable cells in presence of 30 μM S0859 is displayed in Supplemental FIG. 2. Data in panels A (medium+serum), B, C, F and H were compared by unpaired two-tailed Student's t-test, data in panel A (PSS) and G by one-way ANOVA, and data in panel D by linear regression analysis. ***P<0.001, NS: Not significantly different vs. WT under similar conditions (without S0859).

FIG. 13. NBCn1 KO inhibits pHᵢ gradients and filopodia in migrating VSMCs. A. Relative lengths of cellular protrusions at the migrating front of primary aortic explants quantified two hours after scratch infliction. Representative phase contrast images are shown for experiments performed in presence and absence of CO₂/HCO₃⁻ as indicated. The scale bar represents 100 μm, all images are shown at the same magnification. The experiments were performed in medium containing serum (n=6-10). B. Relative lengths of cellular protrusions at the migrating front of primary carotid explants quantified three hours after scratch infliction. The experiments were performed in medium containing serum (n=8-11). C. Relative lengths of cellular protrusions at the migrating front of primary aortic explants quantified two hours after scratch infliction. Experiments were performed in PSS (n=5-11) to avoid binding of S0859 to medium and serum components. D. Fluorescence images of migrating VSMCs recorded two hours after scratch infliction through primary aortic explants from WT and NBCn1 KO mice. From the time of scratch infliction until image acquisition, explants were incubated in presence or absence of CO₂/HCO₃⁻ as specified. The scale bar represents 50 μm, all images are shown at the same magnification. E. Fluorescence image of filopodium and original traces of pHᵢ along filopodia in explants from WT and NBCn1 KO mice. F. Average pHᵢ levels at the tip of filopodia (upper horizontal line) and in the perinuclear cytosolic region (lower horizontal line); n=4-10, each represented by the average of ˜5 filopodia. The height of the bars represents the magnitude of the axial pHᵢ gradient. Acetazolamide (ATZ, 100 μM) was applied to inhibit carbonic anhydrase activity. For the first two hours after scratch infliction, the primary aortic explants were incubated in absence of CO₂/HCO₃⁻ to allow for equivalent filopodia formation in explants from NBCn1 KO and WT mice. Then, explants were exposed to the specified condition (i.e., CO₂/HCO₃⁻, CO₂/HCO₃⁻-free, S0859, ATZ) for 15 minutes prior to initiation of pHᵢ recordings. Data were compared using one-way ANOVA or two-tailed Student's t-tests (paired or unpaired, as appropriate). Bonferroni corrections were applied to adjust for multiple testing. In panel A, B and C: *P<0.05, **P<0.01, ***P<0.001, NS: Not significantly different vs. WT under similar conditions. In panel F: *P<0.05 comparing pHᵢ at tip of filopodia vs. WT with CO₂/HCO₃⁻, **P<0.05 comparing the pHᵢ gradient vs. WT with CO₂/HCO₃⁻, ##P<0.01 comparing the pHᵢ gradient vs. NBCn1 KO with CO₂/HCO₃⁻, NS: not significantly different vs. WT without CO₂/HCO₃⁻.

FIG. 14. Carbonic anhydrase inhibitor acetazolamide promotes wound healing and filopodia in explants from NBCn1 KO mice. A. Progression of wound healing after scratch infliction through primary aortic explants from NBCn1 KO mice with and without 100 μM acetazolamide (ATZ, n=9). Experiments were performed in CO₂/HCO₃⁻-containing medium added serum. B. Original images showing the extent of wound healing over a 3-hour period in primary explants from NBCn1 KO mice in presence or absence of 100 μM acetazolamide. The scale bar represents 200 μm, all images are shown at the same magnification. Experiments were performed in CO₂/HCO₃⁻-containing medium added serum. C. Average relative lengths of cellular protrusions at the migrating front quantified two hours after scratch infliction and original phase-contrast images illustrating the cellular morphology during wound healing in aortic explants from NBCn1 KO mice with or without 100 μM acetazolamide in presence of CO₂/HCO₃⁻. The scale bar represents 100 μm, both images are shown at the same magnification. D. Summary of the association between the axial pHᵢ gradient and the relative rate of wound healing. Open symbols indicate experiments performed in PSS rather than serum-containing medium but otherwise under conditions corresponding to the filled symbols. Data were compared by repeated measures two-way ANOVA (panel A), unpaired two-tailed Student's t-test (panel C) or linear regression analysis (panel D). *P<0.05, ***P<0.001 vs. NBCn1 KO without acetazolamide or as indicated.

FIG. 15. Messenger RNA transcripts for known and putative Na⁺,HCO₃⁻-cotransporters of the Slc4-family are detected in arteries and primary explants from 129/SvJ WT and NBCn1 KO mice. Gel images (representative of three experiments on independent tissue preparations) showing the results of RT-PCR analyses. ‘Positive control’ represents reactions performed on tissue with known expression of the given transporter (NBCn1: kidney medulla, NBCe1: kidney cortex, NBCe2: choroid plexus, NDCBE: cerebral cortex, NCBE/NBCn2: choroid plexus, AE4: kidney cortex, BTR1: kidney medulla). For each sample, a parallel reaction without addition of reverse transcriptase produced no band (not shown) excluding amplification of genomic DNA. Bands of appropriate sizes were sequenced to confirm specificity.

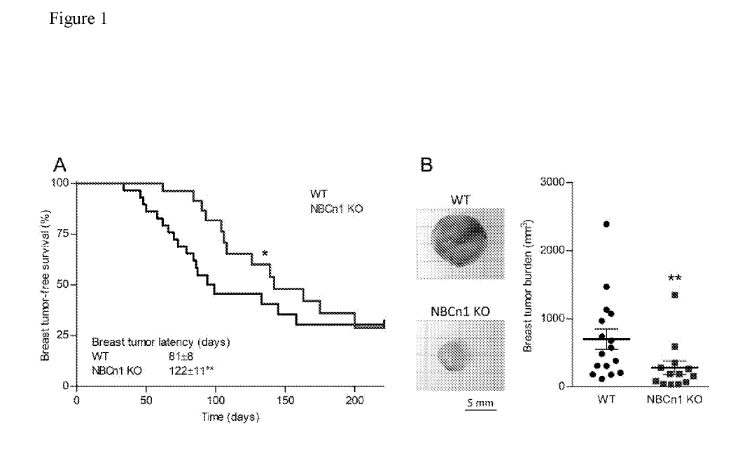

Despite detecting mRNA transcripts for multiple Na⁺,HCO₃⁻-cotransporters of the Slc4-family (NBCn1, NBCe1, NDCBE and BTR1) in arteries and VSMCs from 129/SvJ mice—which is in contrast to our previous findings from mesenteric, coronary and cerebral small arteries of NMRI mice where only NBCn1 was detected at mRNA level⁴—the Na⁺,HCO₃⁻-cotransport was completely abolished in carotid arteries from NBCn1 KO mice (see FIG. 1A-C of the printed article). The finding that NBCn1 is responsible for the Na⁺,HCO₃⁻-cotransport in carotid arteries is consistent with our previous findings from murine resistance-sized mesenteric and middle cerebral arteries.

FIG. 16. S0859 causes loss of cell viability in primary explants. Experiments were performed in CO₂/HCO₃⁻-containing physiological saline solutions (PSS). Data were compared by repeated measures 2-way ANOVA. ***P<0.001.

FIG. 17. Predicted antigenic determinants in extracellular loop 3 of NBCn1. Based on the calculated antigenic index, we selected two overlapping peptides (NBCn1_EL3h1.1 and NBCn1_EL3h2.1) that together cover the regions of high estimated antigenicity.

FIG. 18. Titers of serum samples collected from rabbits immunized with KLH-conjugated peptides corresponding to the predicted extracellular loop 3 of human NBCn1. A+B. Binding of sera from rabbits injected with KLH-conjugated NBCr1_EL3h1.1 (A) or NBCr1_EL3h2.1 (B) to the corresponding BSA-conjugated peptides evaluated by ELISA. The anti-BSA antibody was used as positive control and pre-immune serum served as negative control. Each measurement was made in duplicate from both of the rabbits injected with the respective KLH-conjugated peptide. B1, B2, B3 and B4 refer to serum samples separated from blood drawn at week 7, 8, 10 and 11, respectively.

FIG. 19. The polyclonal antibodies raised against extracellular loop 3 of NBCn1 completely inhibit Na⁺,HCO₃⁻-cotransport activity in MCF-7 human breast cancer cells. A+B. Representative pHᵢ traces in response to NH₄⁺-prepulses and the subsequent recovery with and without the raised antibodies (A) and 30 μM S0859 (B). To improve clarity, the pHᵢ recovery phases (marked by the dotted gray squares) are displayed at twice the magnification of the initial parts of the traces and only the control curve is shown for the period prior to washout of NH₄Cl. The illustrated experiments were performed in the presence of CO₂/HCO₃⁻. The antibodies were added (at 1:100 dilution) at the time of NH₄Cl addition and S0859 was added at the time of NH₄Cl washout and included in all subsequent solutions until the end of the experiment. C+D. Relative initial rates of Na⁺-dependent pHᵢ recovery after intracellular acid-loading with NH₄⁺-prepulses. Equivalent inhibition of net acid extrusion by anti-NBCn1_EL3h2.1 (C, n=11-20) and 30 μM S0859 (D, n=5-6) in the presence of CO₂/HCO₃⁻ supports that the developed antibody completely inhibits Na⁺,HCO₃⁻-cotransport in these cells. This finding is further supported by the finding that anti-NBCn1_EL3h2.1 had no effect in the absence of CO₂/HCO₃⁻ (C, n=6) and the finding from previous studies that siRNA-mediated down-regulation of NBCn1 inhibits net acid extrusion to the same extent as S0859. The remaining net acid-extrusion in the presence of S0859 has been found sensitive to the Na⁺/H⁺-exchange inhibitor 5-(N-ethyl-N-isopropyl)amiloride (Lauritzen et al., 2010). The data regarding S0859 in panels B and D are from (Steinkamp et al., 2015). Data were compared by one-way ANOVA followed by Dunnet's post-tests (experiments with CO₂/HCO₃⁻ in panel C) or by unpaired, two-tailed Student's t-test (panel D and experiments without CO₂/HCO₃⁻ in panel C). *P<0.05, ***P<0.001, NS: not significantly different vs. control under similar CO₂/HCO₃⁻ conditions.

### DETAILED DESCRIPTION OF THE INVENTION

The present invention relates to agents, which are capable of inhibiting the activity of the Na⁺,HCO₃-cotransporter NBCn1 (SLC4A7). The present inventors have surprisingly found that inhibition of NBCn1 mediated HCO₃⁻ transport is associated with reduced growth of breast tumors and increased survival. The invention, therefore, specifically relates to the use of such agent in medicine, in particular in the treatment of cancer, such as breast cancer. NBCn1 is also involved in migration of smooth muscle cells, which is important in restenosis and atherosclerosis; cf. example 2. Therefore, the invention also relates to the use of such agent in the treatment of atherosclerosis and/or restenosis. The agent of the invention may be any suitable agent capable of inhibiting NBCn1 activity and in a preferred embodiment, the agent is an antibody.

Definitions

Antibodies: An antibody according to the invention is a polypeptide or protein capable of recognising and binding an antigen comprising at least one antigen binding site. Said antigen binding site preferably comprises at least one complementarity determining region (CDR). The antibody may be a naturally occurring antibody, a fragment of a naturally occurring antibody or a synthetic antibody.

Naturally occurring antibody: The term “naturally occurring antibody” refers to heterotetrameric glycoproteins capable of recognising and binding an antigen and comprising two identical heavy (H) chains and two identical light (L) chains inter-connected by disulfide bonds. Each heavy chain comprises a heavy chain variable region (abbreviated herein as V_{H}) and a heavy chain constant region (abbreviated herein as C_{H}). Each light chain comprises a light chain variable region (abbreviated herein as V_{L}) and a light chain constant region (abbreviated herein as C_{L}). The V_{H} and V_{L} regions can be further subdivided into regions of hypervariability, termed complementarity determining regions (CDRs), interspersed with regions that are more conserved, termed framework regions (FRs). Antibodies may comprise several identical heterotetramers.

Antigen: Molecule comprising at least one epitope. The antigen may for example be a polypeptide, polysaccharide, protein, lipoprotein or glycoprotein.

Epitope: An epitope is a determinant capable of specific binding to an antibody. Epitopes may for example be comprised within polypeptides, polysaccharide, proteins, lipoproteins or glycoproteins. Epitopes usually consist of chemically active surface groupings of molecules such as amino acids or sugar side chains and usually have specific three dimensional structural characteristics, as well as specific charge characteristics. Epitopes may be conformational or non-conformational, wherein binding to the former but not the latter is lost in the presence of denaturing solvents. Epitopes may be continuous or discontinuous, wherein a discontinuous epitope is a conformational epitope on a protein antigen which is formed from at least two separate regions in the primary sequence of the protein.

Monoclonal Antibody: Monoclonal antibodies (Mab's) are populations of antibodies, wherein every antibody molecule is similar and thus recognises and binds the same epitope. Monoclonal antibodies are in general produced by a host cell line and frequently by a hybridoma cell line. Methods of making monoclonal antibodies and antibody-synthesizing hybridoma cells are well known to those skilled in the art. Antibody producing hybridomas may for example be prepared by fusion of an antibody producing B lymphocyte with an immortalized B-lymphocyte cell line. Monoclonal antibodies according to the present invention may for example be prepared by the standard somatic cell hybridization technique of Kohler and Milstein, Nature 256:495 (1975) or as described in Antibodies: A Laboratory Manual, By Ed Harlow and David Lane, Cold Spring Harbor Laboratory Press, 1988. Said monoclonal antibodies may be derived from any suitable mammalian species, however frequently the monoclonal antibodies will be rodent antibodies for example murine or rat monoclonal antibodies. [symbol] It is preferred that the antibodies according to the present invention are monoclonal antibodies or derived from monoclonal antibodies.

Polyclonal antibodies: Polyclonal antibodies is a population of antibodies comprising a mixture of different antibody molecules recognising and binding a specific given antigen, hence polyclonal antibodies may recognise different epitopes within said antigen. In general polyclonal antibodies are purified from serum of an animal, preferably a mammal, which previously has been immunized with the antigen.5 Polyclonal antibodies may for example be prepared by any of the methods described in Antibodies: A Laboratory Manual, By Ed Harlow and David Lane, Cold Spring Harbor Laboratory Press, 1988.

Antibody or Antigen Binding Fragment

An antibody or antigen binding fragment thereof is provided which is capable of inhibiting cellular NBCn1-mediated Na⁺-dependent HCO₃⁻ transport. An antibody or antigen binding fragment thereof is also provided which specifically recognizes and binds an extracellular polypeptide region of human NBCn1. In a preferred embodiment, the extracellular polypeptide region comprises or consists of a sequence selected from any one of SEQ ID NO: 5, 6, 7, 8, 9, 10, 11, 12, 13, 14, 15 or a fragment thereof. In one particular embodiment, the extracellular polypeptide region comprises or consists of a human sequence selected from any one of SEQ ID NO: 14, 15 or a fragment thereof, such as a fragment of 5-25, such as 10-25, such as 10-20 amino acids thereof. In one embodiment, the antibody or antigen binding fragment thereof is capable of inhibiting cellular NBCn1-mediated Na⁺-dependent HCO₃⁻ transport.

The antibody according to the present invention may be any polypeptide or protein capable of recognising and binding an antigen in a polypeptide region of human NBCn1, preferably an extracellular polypeptide region. Preferably, the antibody is capable of specifically binding said antigen. By the term “specifically binding” is meant binding with at least 10 times higher affinity to the antigen than to a non-specific antigen (e.g. BSA). Typically, the antibody binds with an affinity corresponding to a K_{D} of about 10⁻⁷ M or less, such as about 10⁻⁸ M or less, such as about 10⁻⁹ M or less, for example about 10⁻¹⁰ M or less, or even about 10⁻¹¹ M or even less, when measured as apparent affinities based on 10₅₀ values.

Preferably said antibody is a naturally occurring antibody or a functional homologue thereof. A naturally occurring antibody is a heterotetrameric glycoprotein capable of recognising and binding an antigen comprising two identical heavy (H) chains and two identical light (L) chains inter-connected by disulfide bonds. Each heavy chain comprises or preferably consists of a heavy chain variable region (abbreviated herein as V_{H}) and a heavy chain constant region (abbreviated herein as C_{H}). Each light chain comprises, or preferably consists of, a light chain variable region (abbreviated herein as V_{L}) and a light chain constant region (abbreviated herein as C_{L}). The V_{H} and V_{L} regions can be further subdivided into regions of hypervariability, termed complementarity determining regions (CDRs), interspersed with regions that are more conserved, termed framework regions (FRs). Each V_{H} and V_{L} comprises and preferably consists of three CDRs and four FRs, arranged from amino-terminus to carboxy-terminus in the following order: FR1, CDR1, FR2, CDR2, FR3, CDR3, FR4.

The naturally occurring antibody may also be a heavy-chain antibody (HCAbs) as produced by camelids (camels, dromedaries and llamas). HCAbs are homodimers of heavy chains only, devoid of light chains and the first constant domain (Hamers-Casterman et al., 1993).

Another possibility is New or Nurse Shark Antigen Receptor (NAR) protein, which exists as a dimer of two heavy chains with no associated light chains. Each chain is composed of one variable (V) and five constant domains. The NAR proteins constitute a single immunoglobulin variable-like domain (Greenberg, A. S., Avila, D., Hughes, M., Hughes, A., McKinney, E. C. & Flajnik, M. F. (1995) Nature (London) 374, 168-173.) which is much lighter than an antibody molecule.

Naturally occurring antibodies according to the invention may consist of one heterotetramer or they may comprise several identical heterotetramers. Thus, the naturally occurring antibody according to the invention may for example be selected from the group consisting of IgG, IgM, IgA, IgD and IgE. The subunit structures and three-dimensional configurations of these different classes of immunoglobulins are well known.

Naturally occurring antibodies according to the invention may be antibodies of a particular species, for example the antibody may be a murine, a rat, a rabbit, a goat, a sheep, a chicken, a donkey, a camelid or a human antibody. The antibody according to the invention may however also be a hybrid between antibodies from several species, for example the antibody may be a chimeric antibody, such as a humanised antibody.

The antibody according to the invention may be a monoclonal antibody, such as a naturally occurring monoclonal antibody or it may be polyclonal antibodies, such as naturally occurring polyclonal antibodies.

The antibody may be any protein or polypeptide containing an antigen binding site, such as a single polypeptide, a protein or a glycoprotein. Preferably, the antigen binding site comprises at least one CDR, or more preferably a variable region.

Thus the antigen binding site may comprise a V_{H} and/or V_{L}. In an antibody, the V_{H} and V_{L} together may contain the antigen binding site, however, either one of the V_{H} or the V_{L} may comprise an antigen binding site. In particular, the CDRs may identify the specificity of the antibody and accordingly it is preferred that the antigen binding site comprises one or more CDRs, preferably at least 1, more preferably at least 2, yet more preferably at least 3, even more preferably at least 4, yet more preferably at least 5, even more preferably 6 CDRs. It is preferable that the antigen binding site comprises at least one CDR3, more preferably at least the CDR3 of the heavy chain.

The antibody may for example be an antigen binding fragment of an antibody, preferably an antigen binding fragment of a naturally occurring antibody, a heterospecific antibody, a single chain antibody or a recombinant antibody. An antibody according to the invention may comprise one or more antigen binding sites. Naturally occurring heterotetrameric antibodies comprises two antigen binding sites.

Heterospecific Antibodies

The antibody according to the invention may also be a “heterospecific antibody”, such as a bispecific antibody. A bispecific antibody is a protein or polypeptide, which comprises two different antigen binding sites with different specificities. For example, the bispecific antibody may recognise and bind to two different antigens or it may recognise and bind to two different epitopes within the same antigen. The term “heterospecific antibody” is intended to include any protein or polypeptide, which has more than two different antigen binding sites with different specificities. For example, the heterospecific antibody may recognise and bind to three different antigens or it may recognise and bind to different epitopes on the same antigen. Accordingly, the invention includes, but is not limited to, bispecific, trispecific, tetraspecific, and other multispecific antibodies, which are directed to extracellular polypeptide fragments of human NBCn1.

Bispecific antibodies may for example be prepared starting from monoclonal antibodies, for example by fusing two hybridomas in order to combine their specificity, by chemical crosslinking or using recombinant technologies.

For example the V_{H} and V_{L} of two different antibodies (1 and 2) may be linked by recombinant means to form “cross-over” chains V_{H}1-V_{L}2 and V_{H}2-V_{L}1, and then dimerised to reassemble both antigen-binding sites (see WO 94/09131). Bispecific antibodies may also be prepared by linking two single chain antibodies with different specificity genetically as for example described in WO 94/13806. Also, two antigen binding fragments of an antibody may be linked.

Human and Humanised Antibodies

It is not always desirable to use non-human antibodies for human therapy, and accordingly, the antibody of the invention may be a human antibody or a humanised antibody.

Thus, the antibody according to the invention may be a human or a humanised antibody. A human antibody as used herein is an antibody, which is obtained from a system using human immunoglobulin sequences. Human antibodies may for example be antibodies isolated from an animal (e.g., a mouse) that is transgenic or transchromosomal for human immunoglobulin genes or a hybridoma prepared therefrom. Human antibodies may also be isolated from a host cell transformed to express the antibody, e.g., from a transfectoma. Human antibodies may also be isolated from a recombinant, combinatorial human antibody library.

Human antibodies have variable and constant regions derived from human germline immunoglobulin sequences. In certain embodiments, however, such recombinant human antibodies can be subjected to in vitro mutagenesis or in vivo somatic mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

A human antibody is preferably at least 90%, more preferably at least 95%, even more preferably at least 96%, 97%, 98%, or 99% identical in amino acid sequence to the amino acid sequence encoded by a wild type human immunoglobulin gene.

Said transgenic of transchromosomal animal may contain human immunoglobulin gene miniloci that encode unrearranged human heavy (p and/or y) and K light chain immunoglobulin sequences. Furthermore, the animal may contain one or more mutations that inactivate the endogenous heavy and light chain loci. Examples of such animals are described in Lonberg, N. et al. (1994) Nature 368 (6474):856-859 and WO 02/43478.

The antibody according to the invention may be a chimeric antibody, i.e. an antibody comprising regions derived from different species. The chimeric antibody may for example comprise variable regions from one species of animal and constant regions from another species of animal. For example, a chimeric antibody can be an antibody having variable regions which derive from a mouse monoclonal antibody and constant regions which are human. Such antibodies may also be referred to as humanised antibodies.

Thus, the antibody according to the invention may also be a humanised antibody, which is encoded partly by sequences obtained from human germline immunoglobulin sequences and partly from other sequences. Said other sequences are preferably germline immunoglobulins from other species, more preferably from other mammalian species. In particular a humanised antibody may be an antibody in which the antigen binding site is derived from an immunoglobulin from a non-human species, preferably from a non-human mammal, e.g. from a mouse or a rat, whereas some or all of the remaining immunoglobulin-derived parts of the molecule is derived from a human immunoglobulin. The antigen binding site from said non-human species may for example consist of a complete V_{L} or V_{H} or both or one or more CDRs grafted onto appropriate human framework regions in V_{L} or V_{H} or both. Thus, in a humanized antibody, the CDRs can be from a mouse or rat monoclonal antibody and the other regions of the antibody are of human origin.

Recombinant Antibodies

The antibody according to the present invention may also be a recombinant antibody, i.e. an antibody prepared, expressed, created or isolated by recombinant means.

Single Chain Antibodies

Naturally occurring antibodies are heterotetramers. However, the antibody according to the present invention may also be a single polypeptide comprising one or more antigen binding sites. Such antibodies are also referred to as “single chain antibodies”. Thus the antibody according to the present invention may also be a single chain antibody.

Single chain antibodies may comprise the two domains of the Fv fragment, V_{L} and V_{H}. To obtain such single chain antibodies the genes encoding the V_{L} and V_{H} may be joined, using recombinant methods. Usually they are separated by a synthetic linker, for example a linker of 5 to 100, such as of 5 to 50, for example of 10 to 25 amino acids. Said linker may either connect the N-terminus of the V_{H} with the C-terminus of the V_{L}, or vice versa. This enables production of a single protein chain in which the V_{L} and V_{H} regions pair to form monovalent antibody-like molecules (also known as single chain antibodies or single chain Fv (scFv); see e.g., Bird et al. (1988) Science 242:423-426; and Huston et al. (1988) Proc. Natl. Acad. Sci. USA 85:5879-5883).

The single chain antibody may also be a divalent antibody, e.g. a single peptide chain comprising two V_{H} and two V_{L} regions, which may each be linker by linker. The single chain antibody may also be a multivalent antibody, e.g. a single peptide chain comprising multiple V_{H} and multiple V_{L} regions, which may each be linker by linker. The V_{H} and V_{L} regions may be identical or different, yielding monospecific or heterospecific antibodies, respectively.

Said V_{H} and said V_{L} may be a naturally occurring V_{H} or V_{L}, or a synthetic V_{H} and V_{L} comprising at least one antigen binding site. Preferably said V_{H} and V_{L} are naturally occurring V_{H} and V_{L}.

Antigen Binding Fragments of Antibodies

Antigen binding fragments of antibodies are fragments of antibodies retaining the ability to specifically bind to an antigen. Preferably, said fragment is an antigen binding fragment of a naturally occurring antibody.

It has been shown that the antigen-binding function of an antibody can be performed by fragments of a full-length antibody. Examples of antigen binding fragments of naturally occurring antibodies include for example (i) a Fab fragment, a monovalent fragment consisting of the V_{L}, V_{H}, C_{L} and CH_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody or (v) a dAb fragment (Ward et al., (1989) Nature 341:544-546), which consists of a V_{H} domain. Fab fragments may be prepared by papain digestion. F(ab′)₂ fragments may be prepared by pepsin treatment.

The antigen binding fragment of an antibody preferably comprise at least one complementarity determining region (CDR) or more preferably a combination of two or more isolated CDRs which may optionally be joined by a synthetic linker.

A further example of an antigen binding fragment of an antibody is binding-domain immunoglobulin fusion proteins comprising (i) an antigen binding site fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The antigen binding site can be a heavy chain variable region or a light chain variable region. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

The antigen binding fragment of an antibody may also be diabodies, which are small antibody fragments with two antigen-binding sites. Diabodies preferably comprises a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad Sci. USA 90: 6444-6448 (1993).

Method of Producing Antibody

In general, methods of producing antibodies are well-known to those of skill in the art. Antibodies can be produced using antigenic epitope-bearing peptides or polypeptides derived from NBCn1. Antigenic epitope-bearing peptides and polypeptides of the present invention preferably contain a sequence of at least four, or between 5 and 100, such as between 10 and 100, such as between 15 and 80, for example between 15 and 70, such as between 15 and 60, such as between 15 and 60, such as between 15 and 40, such as between 15 and 30 amino acids contained within NBCn1, such as SEQ ID NO: 1 or 2 or 14 and/or 15. However, peptides or polypeptides comprising a larger portion of NBCn1, for example containing from 100 amino acids and any length up to and including the entire amino acid sequence of NBCn1 are also useful as antigenic epitope-bearing peptides or polypeptides for producing antibodies of the present invention.

The antibodies of the present invention are preferably produced using peptides or polypeptides comprising a region of an extracellular loop of NBCn1 or a fragment thereof, such as at least four, or between 5 and 100, such as between 10 and 100, such as between 15 and 80, for example between 15 and 70, such as between 15 and 60, such as between 15 and 60, such as between 15 and 40, such as between 15 and 30 amino acids thereof. In one embodiment, the antigenic epitope-bearing peptides or polypeptides comprise or consists of:

a) EL2, human and mouse:
(SEQ ID NO: 5)
SPVITFGGLLGEATEGRISAIESLFGASLT
b) EL3, human:
(SEQ ID NO: 6)
KLFDLGETYAFNMHNNLDKLTSYSCVCTEPPNPSNETLAQWKKDNITAHN
ISWRNLTVSECKKLRGVFLGSACGHHGP
C) EL3, mouse:
(SEQ ID NO: 7)
KLFHLGEIYAFNMHNNLDELTSYTCVCAEPSNPSNETLELWKRKNITAYS
VSWGNLTVSECKTFHGMFVGSACGPHGP
d) EL4, human:
(SEQ ID NO: 8)
PSPKLHVPEKFEPTHPERGWIISPLGDNPW
e) EL4, mouse:
(SEQ ID NO: 9)
PSPKLHVPEKFEPTDPSRGWIISPLGDNPW
f) EL5, human and mouse:
(SEQ ID NO: 10)
SISHVNSLKVESECSAPGEQPKFLGIREQR
g) Human:
(SEQ ID NO: 11)
MERFRLEKKLPGPDEEAVVDLGKTSSTVNTKFEKEELESHRAVYIGVHVP
FSKESRRRHRHRGHKHHHRRRKDKESDKEDGRESPSYDTPSQRVQFILGT
EDDDEEHIPHDLFTEMDELCYRDGEEYEWKETARWLKFEEDVEDGGDRWS
KPYVATLSLHSLFELRSCILNGTVMLDMRASTLDEIADMVLDNMIASGQL
DESIRENVREALLKRHHHQNEKRFTSRIPLVRSFADIGKKHSDPHLLERN
GEGLSASRHSLRTGLSASNLSLRGESPLSLLLGHLLPSSRAGTPAGSRCT
TPVPTPQNSPPSSPSISRLTSRSSQESQRQAPELLVSPASDDIPTVVIHP
PEEDLEAALKGEEQKNEENVDLTPGILASPQSAPGNLDNSKSGEIKGNGS
GGSRENSTVDFSKVDMNFMRKIPTGAEASNVLVGEVDFLERPIIAFVRLA
PAVLLTGLTEVPVPTRFLFLLLGPAGKAPQYHEIGRSIATLMTDEIFHDV
AYKAKDRNDLLSGIDEFLDQVTVLPPGEWDPSIRIEPPKSVPSQEKRKIP
VFHNGSTPTLGETPKEAAHHAGPELQRTGRLFGGLILDIKRKAPFFLSDF
KDALSLQC
h) Human:
(SEQ ID NO: 12)
ATVLSISHVNSLKVESECSAPGEQPKFLGIREQRVT
i) Human:
(SEQ ID NO: 13)
DRIKLFGMPAKHQPDLIYLRYVPLWKVHIFTVIQLTC
j) NBCn1_EL3h1.1:
(SEQ ID NO: 14)
[Hz]-HNNLDKLTSYSCVCTEPPNPSNETLAQWKKDNITA-
amide
k) NBCn1_EL3h2.1:
(SEQ ID NO: 15)
[Hz]-LAQWKKDNITAHNISWRNLTVSECKKLRGVFLGSA-
amide.

However, any fragment of any of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15 may be also be used, for example a fragment comprising at least four amino acids, such as between 5 and 75, such as between 10 and 75, such as between 15 and 70, for example between 15 and 60, such as between 15 and 60, such as between 15 and 50, such as between 15 and 40, such as between 15 and 30 amino acids thereof. In a preferred embodiment, the antibodies are produced using peptides comprising an extracellular polypeptide region of NBCn1 and preferably, the extracellular polypeptide region comprises or consists of a sequence selected from any one of SEQ ID NO: 14 and/or 15 or a fragment thereof, such as a fragment of 5-25, such as 10-25, such as 10-20 amino acids thereof.

In a preferred embodiment, the antigenic epitope-bearing peptides or polypeptides are human, such as SEQ ID NO: 5, 6, 8, 10, 11, 12, 13, 14 and 15.

It is desirable that the amino acid sequence of the epitope-bearing peptide is selected to provide substantial solubility in aqueous solvents (i.e., the sequence includes relatively hydrophilic residues, while hydrophobic residues are preferably avoided). Moreover, amino acid sequences containing proline residues may also be desirable for antibody production.

Recombinant antibodies of the invention may for example be produced using a synthetic library or by phage display, preferably using the extracellular domains of NBCn1 to generate antibodies against extracellular domains of NBCn1. Since selection of recombinant antibodies from phage display libraries and synthetic libraries do not build on immunogenicity principles, but solely on binding ability, it is capable of providing antibodies that bind to a more diverse array of epitopes. For example, antibodies binding to synthetic peptides corresponding to surface exposed regions of NBCn1 can be isolated. Next, antibodies binding to cells with high expression levels of NBCn1 may be isolated. The first step may follow any conventional selection protocol (e.g. Mandrup O A, Friis N A, Lykkemark S, Just J and Kristensen P. A novel heavy domain antibody library with functionally optimized complementarity determining regions. PLoS One 8: e76834, 2013), and the second step could utilize any selection procedures (e.g. Sorensen M D, Agerholm I E, Christensen B, Kolvraa S and Kristensen P. Microselection—affinity selecting antibodies against a single rare cell in a heterogeneous population. J Cell Mol Med 14: 1953-1961, 2010; or Sorensen M D and Kristensen P. Selection of antibodies against a single rare cell present in a heterogeneous population using phage display. Nat Protoc 6: 509-522, 2011; or Sorensen M D, Melchjorsen C J, Mandrup O A and Kristensen P. Raising antibodies against circulating foetal cells from maternal peripheral blood. Prenat Diagn 33: 284-291, 2013.). Each selection step produces individual bacterial colonies from which monoclonal (phage) antibodies can be produced. The monoclonal phage antibodies are then typically tested in conventional cell ELISA, where antibody binding is compared between cells expressing high amounts of NBCn1 (e.g., breast cancer cells isolated from WT mice) and cells with no or very low NBCn1 expression (e.g., breast cancer cells isolated from NBCn1 knockout mice). In addition, the antibodies can be tested for binding to purified proteins. Following selection, the top monoclonal phage antibody fragments can be cloned into expression vectors, which comprise the constant regions of the IgG or different expression tags.

Recombinant antibodies may be produced in microbial host organisms, such as bacteria, yeast or cell cultures of cells derived from multicellular organisms. Frequently, Escherichia coli is useful as host organism. Frequently, recombinant antibodies are fragments of naturally occurring antibodies comprising at least one antigen binding site, such as a Fab fragment, a Fv fragment or the recombinant antibody is a scFV.

Recombinant antibodies may be identified using various systems, such as phage display or ribosome display. In a preferred embodiment, the present invention relates to methods of producing antibodies targeting extracellular regions of NBCn1 using phage display. The starting point of phage display is usually a library of antibodies, such as single chain antibodies or fragments of naturally occurring antibodies expressed by a phage. Various different kinds of phages are suitable for use in phage display, e.g. M13, fd filamentous phage, T4, T7 or λ phage. Phagemids may also be used, but that usually requires use of a helper phage. Typically, the library comprises in the range of 10⁷ to 10¹⁵, such as 10⁹ to 10¹¹ different phages. The antibodies may be either of native or immune origin. The antibodies of the library may be fused to a phage coat protein (e.g. g3p or g8p) in order to ensure display on the surface. Thus, the antibody (fragment) may be encoded by a nucleic acid sequence, which is cloned upstream or downstream of a nucleic acid encoding a phage coat protein, which is operably linked to a suitable promoter.

The genomic information coding for antibody e.g. for the antibody variable domains may be obtained from B cells of nonimmunised or immunised donors using recombinant DNA technology to amplify the VH and VL gene segments and cloning into an appropriate phage. Synthetic libraries may be prepared by rearranging VH and VL gene segments in vitro and/or by introducing artificial sequences into VH and VL gene segments. For example synthetic libraries may be prepared using a VH and VL gene framework, but introducing into this artificial complementarity determining regions (CDRs), which may be encoded by random oligonucleotides.

The library may also be different libraries, which are then combined in the host cell. Thus, one library may comprise heavy chain sequences, such as the heavy chain Fv fragment or Fab fragment or VH and the other light chain sequences, such as the light chain Fv fragment or Fab fragment VL.

Typically, several rounds of selection, e.g. 2, 3, 4, 5 or 5, such as 2 to 5 or 2 to 4 or 2 to 3 rounds of selection are performed. This may be done by immobilising the antigen, contacting the antigen with the phage and isolation of bound phages. The antigen may be immobilised on any suitable solid surface, such as a plastic surface, beads (such as magnetic beads)

In one aspect, the present invention relates to a method of selecting antibodies, said method comprising the steps of
a) Providing an antibody library, preferably a bacteriophage library, which may comprise in the range of 10⁷ to 10¹⁵, such as 10⁹ to 10¹¹, different phages
b) Providing one or more NBCn1 antigens, preferably an antigen of an extracellular region of NBCn1,
c) Contacting said antibody library with said one or more NBCn1 antigens, and
d) Selecting antibodies, which bind said one or more NBCn1 antigens.

The method may comprise additional rounds of selection, such as 2, 3, 4, 5, 5 rounds of selection or more, where a library consisting of the selected antibodies in step d) are contacted with an NBCn1 antigen and again selecting antibodies, which bind the NBCn1 antigen. Thus, in one embodiment, the method comprises repeating steps a)-d) once or twice or more, preferably 1, 2, 3 times, where the antibody library in step a) of additional rounds corresponds to the antibodies selected in step d) of the previous round.

The antibodies of the library are in one embodiment selected for their ability to bind peptides or polypeptide fragments of NBCn1 as described elsewhere herein, in particular extracellular fragments (e.g. a polypeptide region comprising a sequence selected from any one of SEQ ID NO: 5-10 or SEQ ID NO: 14-15 or a fragment thereof), or the full-length NBCn1, preferably natively folded NBCn1. In a preferred embodiment, the antigens are immobilized on a solid surface, for example on solid beads.

The provided NBCn1 antigen may be provided as epithelial organoids, for example, epithelial organoids of breast cancer tissue. Thus, in one embodiment, the antigens are provided as epithelial organoids and suitable antibodies are selected by their binding ability to antigens presented on these epithelial organoids. For example, specific antibodies can be identified based on the different binding abilities to epithelial organoids of breast cancers from NBCn1 KO and WT mice.

Polyclonal antibodies to recombinant protein or isolated from natural sources can be prepared using methods well-known to those of skill in the art. See, for example, Green et al., “Production of Polyclonal Antisera,” in Immunochemical Protocols (Manson, ed.), pages 1 to 5 (Humana Press 1992), and Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995). The immunogenicity of a polypeptide can be increased through the use of an adjuvant, such as alum (aluminum hydroxide) or Freund's complete or incomplete adjuvant. Polypeptides useful for immunization also include fusion polypeptides, such as fusions of or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an antibody specific for a polypeptide according to the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310 (1990).

In one aspect of the invention, a method is provided for producing an antibody specifically recognizing and binding an extracellular polypeptide region of human NBCn1, wherein said antibody is capable of inhibiting cellular NBCn1-mediated Na⁺-dependent HCO₃⁻ transport, said method comprising the steps of

a) administering to an animal, preferably a mammal, such as horses, cows, dogs, chicken, rats, mice, rabbits, guinea pigs, goats, or sheep, an extracellular polypeptide fragment of human NBCn1 (e.g. as defined by any of SEQ ID NO: 3-10 or 14-15) or a homolog at least 90% identical thereto, such as at least 91, 92, 93, 94, 95, 96, 97 or at least 98% or 99% identical thereto,

b) isolating antibodies from said mammal,

c) testing whether antibodies are capable of inhibiting cellular NBCn1-mediated Na⁺-dependent HCO₃⁻ transport and/or testing whether such antibodies are capable of specifically recognizing and binding an extracellular polypeptide region of human NBCn1 and

d) selecting antibodies capable of inhibiting cellular NBCn1-mediated Na⁺-dependent HCO₃⁻ transport and/or selecting antibodies capable of specifically recognizing and binding an extracellular polypeptide region of human NBCn1.

Alternatively, monoclonal antibodies specific for polypeptides according to the present invention can be generated. Rodent monoclonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.1 2.6.7 (John Wiley & Sons 1991) [“Coligan”], Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice or rabbits with a composition comprising a gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an antibody specific for polypeptides according to the present invention of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1 2.7.12 and pages 2.9.1 2.9.3; Baines et al., “Purification of Immunoglobulin G (IgG),” in Methods in Molecular Biology, Vol. 10, pages 79 104 (The Humana Press, Inc. 1992)).

For particular uses, it may be desirable to prepare fragments of antibodies specific for extracellular regions of NBCn1. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an F_{c} fragment directly. These methods are described, for example, by Goldenberg, U.S. Pat. No. 4,331,647, Nisonoff et al., Arch Biochem. Biophys. 89:230 (1960), Porter, Biochem. J. 73:119 (1959), Edelman et al. and Coligan, both in Methods in Enzymology Vol. 1, (Academic Press 1967).

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Nat'l Acad. Sci. USA 69:2659 (1972). Alternatively, the variable chains can be linked by an intermolecular disulfide bond or cross-linked by chemicals such as glutaraldehyde (see, for example, Sandhu, Crit. Rev. Biotech. 12:437 (1992)).

The Fv fragments may comprise V_{H} and V_{L} chains, which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97 (1991) (also see, Bird et al., Science 242:423 (1988), Ladner et al., U.S. Pat. No. 4,946,778, Pack et al., Bio/Technology 11:1271 (1993), and Sandhu, supra).

As an illustration, a scFV can be obtained by exposing lymphocytes to polypeptide in vitro, and selecting antibody display libraries in phage or similar vectors (for instance, through use of immobilized or labeled protein or peptide). Genes encoding polypeptides having potential polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides, which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the sequences disclosed herein to identify proteins which bind to.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106 (1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an antibody specific for a polypeptide according to the present invention may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986), Carter et al., Proc. Nat'l Acad. Sci. USA 89:4285 (1992), Sandhu, Crit. Rev. Biotech. 12:437 (1992), Singer et al., J. Immun. 150:2844 (1993), Sudhir (ed.), Antibody Engineering Protocols (Humana Press, Inc. 1995), Kelley, “Engineering Therapeutic Antibodies,” in Protein Engineering: Principles and Practice, Cleland et al. (eds.), pages 399 434 (John Wiley & Sons, Inc. 1996), and by Queen et al., U.S. Pat. No. 5,693,762 (1997).

Polyclonal anti-idiotype antibodies can be prepared by immunizing animals with antibodies or antibody fragments specific for a polypeptide according to the present invention, using standard techniques. See, for example, Green et al., “Production of Polyclonal Antisera,” in Methods In Molecular Biology: Immunochemical Protocols, Manson (ed.), pages 1 12 (Humana Press 1992). Also, see Coligan at pages 241 to 247. Alternatively, monoclonal anti-idiotype antibodies can be prepared using antibodies or antibody fragments specific for a polypeptide according to the present invention as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J. Gen. Virol. 77:1875 (1996).

Analysis of Antibody

The antibodies or antigen binding fragments provided herein specifically recognize and bind human NBCn1, in particular an extracellular polypeptide region of human NBCn1, such as a sequence selected from any one of SEQ ID NO: 5-15 or a fragment thereof. Relevant fragments are described elsewhere herein.

Such antibodies and antigen binding fragment thereof can be produced by methods described elsewhere herein. Suitable antibodies and antigen binding fragment thereof can be tested in a number of different assays. In particular, the binding abilities of an isolated antibody or antigen binding fragment thereof can be determined in an ELISA assay. Thus, in one embodiment, the antibody or antigen binding fragment thereof provided herein is capable of displacing one or more reference antibodies in a competitive ELISA assay.

Moreover, suitable antibodies and antigen binding fragment thereof can be analysed by immunoblotting, typically where isolated organoid membranes are incubated with the candidate antibody as primary antibody, and following washing detected by incubating with a detectable secondary antibody, which is typically conjugated to horseradish peroxidase for detection.

Suitable antibodies and antigen binding fragment thereof can also be analysed by Immunohistochemistry. For example paraffin-embedded sections of breast tissue can be used and incubated with the candidate antibodies for NBCn1, and antigen binding can subsequently be detected by incubation with a detectable secondary antibody, which is typically conjugated to horseradish peroxidase.

The function of suitable antibodies or antigen binding fragment thereof may also be determined on the basis of their ability to inhibit Na⁺-dependent HCO₃⁻ transport. Thus, in one embodiment, the antibody or antigen binding fragment thereof provided herein is capable of inhibiting cellular NBCn1-mediated Na⁺-dependent HCO₃⁻ transport. One approach could be to test the ability to inhibit NBCn1-mediated Na⁺-dependent HCO₃⁻ transport in cells, for example in cancer cells, such as epithelial breast cancer organoids. NBCn1 is involved in maintaining cellular pH and in particular in avoiding low pH in cancer cells. Therefore, the function of antibodies and antigen binding fragments thereof in recognizing and binding NBCn1 and inhibiting cellular Na⁺-dependent HCO₃⁻ transport can be determined by measuring intracellular pH in human cells and for example mice cells from NBCn1 KO vs. WT mice. Intracellular pH can also be determined in human cancer cell lines, such as MCF7. In particular, the intracellular pH may be determined in organoids from human and/or mice NBCn1 KO vs. WT, preferably in epithelial breast cancer organoids. Antibodies or antigen binding fragments thereof, for which the intracellular pH is decreased when supplied to cells or organoids, are functional as NBCn1 inhibitors, and are consequently suitable for use in medicine, in particular in anticancer therapy as described elsewhere herein.

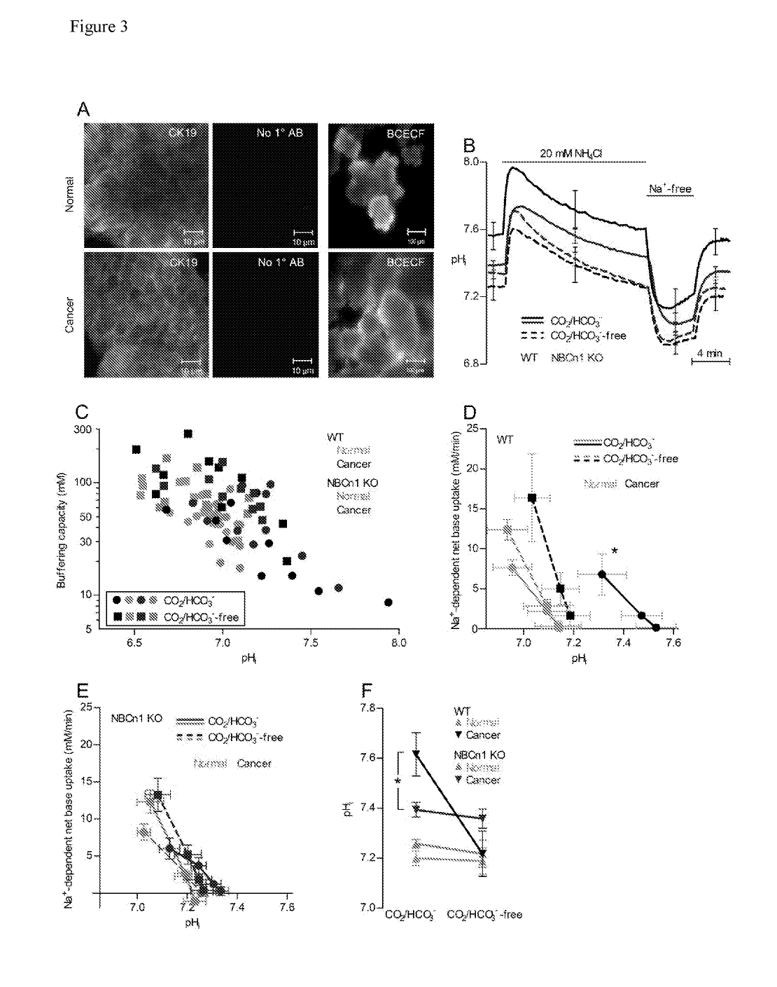

Intracellular pH (pHᵢ) can be determined as described in the example. In particular, pHᵢ can be determined as recovery of pHᵢ from acidosis in isolated organoids loaded with BCECF-AM. Epifluorescence is typically measured with a camera-based fluorescence imaging system during alternating excitation at approximately 485 and 440 nm. The 485/440 BCECF fluorescence ratio can be converted to pHᵢ using the high-[K⁺] nigericin calibration technique. Intracellular acidification can be induced with the NH₄⁺ prepulse technique (traces are shown in FIG. 3B), and acid-base transport activities calculated as the product of the pHᵢ recovery rate and the buffering capacity of that same organoid. Intracellular buffering capacity can be calculated based on the change in pHᵢ upon washout of NH₄Cl with the assumption that NH₃ is in equilibrium across cell membranes.

Medical Use and Treatment

The antibodies and compositions of the present invention are particularly useful in medicine and in therapeutic methods. In one aspect, the invention relates to an antibody of the present invention or antigen binding fragment thereof for use in medicine. In particular, the invention provides an antibody of the present invention or antigen binding fragment thereof for use in the treatment of cancer, in particular breast cancer. In fact, the antibody or antigen binding fragment thereof may be used for the treatment of any hyperproliferative disorder, including cancers, hyperproliferative/inflammatory skin conditions, such as psoriasis and other eczemas, and hyperproliferative disorders of the stomach and gastrointestinal tract.

The antibody or antigen binding fragment thereof may also be used in the treatment of atherosclerosis or restenosis.

In one aspect, the invention relates to the use of an antibody of the present invention or antigen binding fragment thereof in the manufacture of a medicament. In particular, the invention provides the use of an antibody of the present invention or antigen binding fragment thereof in the manufacture of a medicament for the treatment of any hyperproliferative disorder, preferably cancer, in particular breast cancer, or for the treatment of atherosclerosis or restenosis.

The invention also provides a method for treatment of a clinical condition, such as any hyperproliferative disorder, preferably cancer, in particular breast cancer, or atherosclerosis or restenosis, said method comprising administering a therapeutically effective amount of an antibody of the present invention or antigen binding fragment thereof to a subject in need thereof.

The uses, medicaments and methods of treatment are preferably intended for mammalian subjects and in particular human subjects.

The uses, medicaments and methods of treatment can be directed towards any cancer and in a preferred embodiment, the uses, medicaments and methods are directed to the treatment of breast cancer, atherosclerosis and/or restenosis. In particular, the treatment can be directed to those having a triple-negative breast cancer, which refers to breast cancers that do not express the genes for estrogen receptor (ER), progesterone receptor (PR) and Her2/neu. These cancers are difficult to treat since most available chemotherapies target one of the three receptors. However, in another embodiment, the medical uses and methods of treatment of the invention are directed towards HER2-positive breast cancers. Overexpression of HER2 in breast cancer cells is associated with a strong increase in the expression of NBCn1. In yet another embodiment, however, the medical uses and methods of treatment of the invention are directed towards estrogen receptor-positive breast cancers.

The treatment can be applied at any stage of a breast cancer and can also be used for prophylactic treatment of breast cancer. Thus, the treatment according to the present invention involves both prophylactic treatment as well as curative treatment, prevention and amelioration of symptoms associated with a clinical condition, such as cancer, in particular breast cancer.

The uses, medicaments and methods of treatment of the present invention may in addition to an antibody of the present invention or antigen binding fragment thereof involve at least one additional agent. For example, an additional agent could be an anticancer agent, in particular an agent suitable for use in breast cancer, such as a Her2 antibody. Other such suitable additional agents includes regulators of ion transport, such as Na⁺,H⁺-exchangers (NHEs), monocarboxylic acid transporters (MCTs) and proton pump inhibitors (PPIs).

When the treatment concerns atherosclerosis and/or restenosis, the additional agent may be chosen from active agents suitable for the treatment of atherosclerosis and/or restenosis. For example, statins (HMG-CoA reductase inhibitors) could be used as such additional agents, including Atorvastatin, Cerivastatin, Fluvastatin, Lovastatin, Mevastatin, Pitavastatin, Pravastatin, Rosuvastatin and/or Simvastatin.

The uses, medicaments and methods of treatment could be combined with chemotherapy and/or radiotherapy. The uses, medicaments and methods of treatment may also be used before or after surgical therapy.

Inhibition of Na⁺-Dependent HCO₃⁻ Transport

Generally, the present invention provides methods for inhibiting Na⁺-dependent HCO₃⁻ transport mediated by NCBn1. Thus, in one aspect, a method is provided for inhibiting cellular NBCn1-mediated Na⁺-dependent HCO₃⁻ transport, said method comprising providing a sufficient amount of an antibody of the present invention or an antigen-binding fragment thereof to target cells. This method is in one embodiment an in vitro method. The target cells are preferably a population of cancer cells, in particular a population of breast cancer cells, such as epithelial breast cancer.

Administration

In general, suitable methods of administering antibodies are well-known in the art. Thus, any suitable route of administration may be employed for providing a mammal, especially a human, with an effective dose of an antibody or antigen binding fragment thereof as provided herein. For example, oral, rectal, vaginal, topical, parenteral, ocular, pulmonary, nasal, and the like may be employed. Other examples of administration include sublingual, intravenous, intramuscular, intrathecal, subcutaneous, cutaneous and transdermal administration. In one embodiment the administration comprises inhalation, injection or implantation. The administration of the compound according to the present invention can result in a local (topical) effect or a body-wide (systemic) effect. In a preferred embodiment, the antibody or antigen binding fragment thereof is administered by intravenous injection/infusion.

Dosage forms include tablets, troches, dispersions, suspensions, solutions, capsules, creams, ointments, aerosols, and the like. Preferably compounds of the invention are administered orally or intravenously. The effective dose employed of antibody or antigen binding fragment thereof may vary depending on the particular compound, the mode of administration, the condition being treated and the severity of the condition being treated. Such dosage may be ascertained readily by a person skilled in the art.

In one embodiment the antibody or antigen binding fragment thereof of the present invention is administered at a dosage of from about 0.1 milligram to about 100 milligram per kilogram of body weight. For most large mammals, the total dosage is from about 1.0 milligrams to about 1000 milligrams, preferably from about 1 milligram to about 50 milligrams. In the case of a 70 kg adult human, the total dose will generally be from about 1 milligram to about 350 milligrams. For a particularly potent compound, the dosage for an adult human may be as low as 0.1 mg. The dosage regimen may be adjusted within this range or even outside of this range to provide the optimal therapeutic response.

The weekly dosage of antibody or antigen binding fragment thereof is preferably 4-8 mg/kg bodyweight. However, weekly dosage may vary over the course of treatment. Thus, an effective dose of antibody or antigen binding fragment thereof may be administered as an initial loading dose of 4-8 mg/kg bodyweight and subsequent doses of 1-5 mg/kg bodyweight, where e.g. the initial loading dose is administered in week 1, and the subsequent doses are administered weekly in the following weeks.

A complete course of treatment should preferably result in a total dose of 20-300 mg/kg bodyweight; more preferred 50-200 mg/kg bodyweight, such as about 110 mg/kg bodyweight regardless of the dosing regimen employed

The effective dose is preferably infused intravenously, for example by infusion in 5-180 minutes, preferably between 10 and 120 minutes, such as between 30 and 90 minutes.

Composition

The present invention relates to an antibody or antigen binding fragment thereof specifically recognizing and binding an extracellular polypeptide region of human NBCn1 for use in medicine. When used in medicine, the antibody or antigen binding fragment thereof is usually provided as a composition, which in addition to the active agent, i.e. said antibody or antigen binding fragment thereof, comprises one or more additional components. Thus, in one aspect, the present invention relates to a composition, such as a pharmaceutical composition, comprising at least one antibody or antigen binding fragment thereof specifically recognizing and binding an extracellular polypeptide region of human NBCn1, as described elsewhere herein. The composition may comprise a combination of two or more such antibodies.

The pharmaceutical composition also preferably comprises a pharmaceutically acceptable excipient or carrier. The compositions may be prepared for parenteral administration, particularly in the form of liquid solutions or suspensions in aqueous physiological buffer solutions; for oral administration, particularly in the form of tablets or capsules; or for intranasal administration, particularly in the form of powders, nasal drops, or aerosols.Compositions for other routes of administration may be prepared as desired using standard methods.

Formulations for parenteral administration may contain as common excipients (i.e., pharmaceutically acceptable carriers) sterile water or saline, polyalkylene glycols such as polyethylene glycol, oils of vegetable origin, hydrogenated naphthalenes, and the like.ln particular, biocompatible, biodegradable lactide polymer, lactide/glycolide copolymer, or polyoxethylene-polyoxypropylene copolymers are examples of excipients for controlling the release of a compound of the invention in vivo.Other suitable parenteral delivery systems include ethylene-vinyl acetate copolymer particles, osmotic pumps, implantable infusion systems, and liposomes.Formulations for inhalation administration may contain excipients such as lactose, if desired.lnhalation formulations may be aqueous solutions containing, for example, polyoxyethylene-9-lauryl ether, glycocholate and deoxycholate, or they may be oily solutions for administration in the form of nasal drops.If desired, the compounds can be formulated as gels to be applied intranasally. Formulations for parenteral administration may also include glycocholate for buccal administration

The composition may also comprise at least one additional active agent, such as another agent suitable for the treatment of cancer, in particular breast cancer, such as an additional anti breast cancer agent, a Na⁺,H⁺-exchanger (NHE), a monocarboxylic acid transporter (MCT) or a proton pump inhibitor (PPI).

### EXAMPLES

The present example shows that disruption of NBCn1 (Slc4a7) delays murine breast cancer development and establishes NBCn1 as a target for anti-cancer therapy. It is shown that NBCn1 maintains pHᵢ of breast cancer tissue in a neutral to alkaline range that facilitates cancer development and progression.

### Example 1

Methods

Mouse Model and Tumor Induction

NBCn1 KO mice were generated and backcrossed for at least 10 generations with WT C57BL/6j mice. Breast cancer was induced using a well-established model of breast carcinogenesis by implanting medroxyprogesterone acetate pellets (MPA, 50 mg, 90 days release; Innovative Research of America, FL, USA) subcutaneously in 6 weeks old female mice, and treating them with 1 mg dimethyl benz(a)anthracene (DMBA, in 100 μL cottonseed oil) by gavage at 9, 10, 12 and 13 weeks of age. Hormone pellets were replaced 90 and 180 days after first implantation unless tumors were detected prior to these dates. The treated mice were examined for tumor development by thorough bi-weekly palpations.

Microdialysis Sampling and Analyses

Mice were anaesthetized with an intraperitoneal injection of ketamine and xylazine on day 14 after first tumor detection. Microdialysis probes (CMA 20 Elite, CMA Microdialysis AB, Sweden) with 4 mm membrane length were placed in breast tumors and matched normal breast tissue of the same mice guided by a steel needle and split tubing. The probes were perfused by Harvard Pump 33 dual syringe pumps (Harvard Apparatus, MA, USA) at a rate of 0.5 μL/min. From each probe, 6 μL dialysate was collected and analyzed for [glucose], [lactate], [pyruvate] and [glycerol] using a CMA 600 Microdialysis Analyzer (Sweden).

Tumor Latency, Growth Rate and Aggressiveness Score

Tumor latency was defined as the time from last oral gavage with DMBA to first tumor detection. At the end of the microdialysis procedure, mice were euthanized and tumor size(s) measured using calipers to determine tumor growth rate during the first 14 days after tumor detection. Total tumor burden was determined as the sum of individual tumor volumes (V) calculated as V=W²×L×π/6, where W is tumor width and L is tumor length. A composite “aggressiveness score” was calculated as the ratio between tumor burden and tumor latency.

Histopathology

Excised tumor parts and matched normal breast tissue were fixed for 30-60 minutes in 4% neutral-buffered formaldehyde (VWR, Denmark), paraffin embedded and cut to 3 μm thick sections. Deparaffinized and rehydrated slides were stained with hematoxylin and eosin. The histopathology of the breast tumors was evaluated and categorized by an experienced pathologist blinded for genotype.

Breast Epithelial Organoids

Epithelial organoids were isolated from primary breast cancer and matched normal breast tissue: Tissue samples were cut into 1 mm large pieces in phosphate-buffered saline (PBS, containing (in mM): 154.2 Na⁺, 4.1 K⁺, 140.6 Cl⁻, 8.1 HPO₄²⁻, and 1.5 H₂PO₄⁻; pH 7.4) and transferred to T25 culture flasks containing advanced DMEM/F12 culture medium (Life Technologies, Denmark) supplemented with 10% fetal bovine serum (Biochrom AG, Germany)and a final concentration of 450 IU/mL collagenase type 3 (Worthington Biochemical Corporation, NJ, USA). The culture flasks were then transferred to a shaking incubator (at ˜60 revolutions per minute) and left for 4 hours in an atmosphere of 5% CO₂ at 37° C. Next, portions of the tissue suspensions were transferred to Eppendorf tubes and organoids were allowed to sediment for 20 minutes in the incubator without shaking. The epithelial nature of the breast organoids was confirmed by staining with the epithelial cell marker CK-19 and the myofibroblast marker α-SMA (FIG. 3A). To avoid culture-induced changes in cell function or protein expression patterns, all procedures were performed on freshly isolated organoids directly without cell culture.

Intracellular pH Measurements

Recovery of pHᵢ from acidosis was studied in freshly isolated organoids loaded for 20 minutes with 5 μM BCECF-AM. Epifluorescence was collected at 510 nm with a camera-based EasyRatioPro fluorescence imaging system (Photon Technology International, NJ, USA) during alternating excitation at 485 and 440 nm. The 485/440 BCECF fluorescence ratio was converted to pHᵢ using the high-[K⁺] nigericin calibration technique. Intracellular acidification was induced with the NH₄⁺ prepulse technique (traces are shown in FIG. 3B), and acid-base transport activities calculated as the product of the pHᵢ recovery rate and the buffering capacity. Assuming that NH₃ is in equilibrium across cell membranes, intracellular intrinsic buffering capacities were calculated from the change in pHᵢ upon washout of NH₄Cl in absence of CO₂/HCO₃⁻.

Contribution from CO₂/HCO₃⁻ to intracellular buffering capacity was calculated using the equation β_{CO}_{₂}_{/HCO}_{₃}_{^{−}}=2.3×[HCO₃⁻]ᵢ. NH₄Cl was initially washed out into a Na⁺-free solution, and the rate of Na⁺-dependent net base uptake was calculated based on the increase in pHᵢ recovery rate upon subsequent re-addition of bath Na⁺. The CO₂/HCO₃⁻-containing salt solution used for pHᵢ recordings had the following composition (in mM): 140 Na⁺, 4 K⁺, 1.6 Ca²⁺, 1.2 Mg²⁺, 122 Cl⁻, 24 HCO₃²⁻, 1.2 SO₄²⁻, 1.18 H₂PO₄⁻, 10 HEPES, 5.5 glucose, and 0.03 EDTA. In Na⁺-free solutions, Na⁺ was substituted with an equimolar amount of N-methyl-D-glucammonium (NMDG⁺), except for NaHCO₃, which was replaced with choline-HCO₃. In HCO₃-containing solutions at pH 6.8, [HCO₃⁻] was reduced to 6 mM by substitution with Cl⁻. In HCO₃⁻-free solutions, HCO₃⁻ was substituted with an equimolar amount of Cl⁻. HCO₃⁻-containing solutions were aerated with a gas mixture of 5% CO₂ balance air, HCO₃⁻-free solutions were gassed with atmospheric air (nominally CO₂-free); pH was adjusted to 7.4 or 6.8 at 37° C. All solutions contained 5 mM probenecid to inhibit cellular extrusion of BCECF by the organic anion transporter.

Antibodies

Primary antibodies against the following proteins were purchased from Cell Signaling Technology (MA, USA): Akt (#9272, diluted 1:200), Ser473 phospho-Akt (#4060, diluted 1:200), Thr172 phospho-AMPKα (#2535, diluted 1:1000), ERK1/2 (#9102, diluted 1:200), Thr202+Tyr204/Thr185+Tyr187 phospho-ERK1/2 (#9101, diluted 1:200), Tyr1221+1222 phospho-ErbB2 (#2243, diluted 1:200), and PARP-1 (#9542, diluted 1:500). Anti-NHE1 (#sc-136239, diluted 1:500) and anti-MCT4 (#sc-50329, diluted 1:2000) antibodies were from Santa Cruz Biotechnology (TX, USA), anti-MCT1 (#AB3538P, diluted 1:500) from Millipore (Denmark), anti-β-actin (#A5441, diluted 1:5,000) from Sigma-Aldrich (Denmark), anti-p150^{Glued} (#610473, diluted 1:1,000) from BD Transduction Laboratories (Denmark) and anti-CK-19 (#ab15463, diluted 1:100), anti-α-SMA (#ab112022, 1:200), anti-Ki-67 (#ab16667, diluted 1:50) and anti-pHH₃ (#ab5176, diluted 1:800) from Abcam (UK). The anti-NBCn1 antibody, generously provided by Dr. Jeppe Praetorius, Aarhus University, was diluted 1:600, and the anti-AMPKα1 antibody (43), kindly donated by Dr. Grahame Hardie, University of Dundee, was diluted 1:10,000. Anti-mouse (#A1293, diluted 5,000), anti-rabbit (#A3937, diluted 1:5,000) and anti-sheep (#A5187, diluted 1:5,000) alkaline phosphatase-conjugated secondary antibodies were from Sigma-Aldrich. The horseradish peroxidase conjugated anti-rabbit secondary antibody (#7074, diluted 1:1000 (for Ki-67) or 1:2000 (for pHH₃)) was from Cell Signaling Technology and the Alexa Fluor® 488 coupled goat anti-rabbit secondary antibody (#A-11008) from Life Technologies.

Immunoblotting

Organoids isolated from primary breast cancer and matched normal breast tissue were snap frozen in liquid N₂, stored at −80° C. and lysed. Protein concentrations were determined for each sample with the RC DC protein assay (Bio-Rad Laboratories, Denmark). Concentrations of extracted protein were equalized with double-distilled H₂O and samples mixed with NuPAGE LDS Sample Buffer (Invitrogen, #NP0007) and dithiothreitol (DTT). Equal amounts of protein were loaded onto each lane of precast NuPAGE 10% Bis-Tris gels (NOVEX by Life Technologies, #NP0302BOX), separated by sodium dodecyl sulphate-polyacrylamide gel electrophoresis (SDS-PAGE) under denaturing and reducing conditions using a NOVEX system and NuPAGE MOPS SDS Running Buffer (NOVEX by Life Technologies, #NP0001). Benchmark protein ladder (Invitrogen, #10747-012) was used for identification of protein sizes. The separated proteins were transferred to nitrocellulose membranes (Invitrogen, #LC2000) in an XCel II Blot Module (Invitrogen, #E19051) using NuPAGE Transfer Buffer (NOVEX by Life Technologies, #NP0006). The nitrocellulose membranes were stained with Ponceau S Solution (Sigma-Aldrich, #P7170-1L), blocked in blocking buffer (5% nonfat dry milk in TBST: 0.01 M Tris/HCl, 0.15 M NaCl, 0.1% Tween 20) for 1 hour at 37° C. and incubated overnight at 4° C. with primary antibodies diluted in blocking buffer. The membranes were washed 1×15 minutes and 3×5 minutes in TBST and incubated for 1-2 hours at room temperature with alkaline phosphatase-conjugated secondary antibodies diluted in blocking buffer. The membranes were washed in TBST and developed using BCIP/NBT Phosphatase Substrate (KPL, MD, USA; #50-81-08). The membranes were scanned and the intensity of the bands quantified using UN-SCAN-IT 6.1 (Silk Scientific, UT, USA). Individual band intensities were normalized to the average level for that membrane and expressed relative to the mean expression level for the normal breast tissue from WT mice.

Immunohistochemistry and Whole-Mount Immunofluorescence

Mitotic activity was evaluated based on immunohistochemical detection of Ki-67 and pHH₃ expression. Excised breast tissue was fixed in neutral-buffered 4% formaldehyde for 30-60 minutes, paraffin-embedded and cut to 3 μm thick sections. Sections were heated at 80° C. for 1 hour, then deparaffinized in xylene and rehydrated by graded alcohol rinses. Endogenous peroxidase activity was blocked by incubation in 3% H₂O₂ for 20 minutes. Heat-induced antigen retrieval was performed by microwave heating at 600 Win 10 mM citrate buffer (pH 6.0) for 2×10 minutes followed by blocking of non-specific sites with 1% bovine serum albumin (Ki-67) or 10% goat serum (pHH₃) in PBS. Following incubation with primary antibodies (diluted in PBS with 1% bovine serum albumin and 0.1% Triton X-100) for 24 hours (Ki-67) or overnight (pHH₃) at 5° C., the preparations were washed vigorously and incubated with species-matched secondary antibodies for 60 minutes at room temperature. Color development was achieved using 3,3′-diaminobenzidine (DAB; DAKO, Denmark), and slides were counterstained with hematoxylin. Ki-67 and pHH₃ labeling indices were calculated as the fraction of cells positive for Ki-67 and pHH₃, respectively. Only glandular structures were evaluated in the normal breast tissue. Cell counts were obtained using ImageJ software (Rasband; NIH, MD, USA) by investigators blinded for NBCn1 genotype.

Expression of CK-19 and α-SMA was evaluated by whole-mount immunofluorescence imaging.

Statistics

Data are expressed as mean±SEM, unless otherwise specified. Single variables were compared between groups by unpaired two-tailed Student's t-test. One-sample t-tests were performed to determine if the means of single distributions were different from hypothetical values. We used regular or repeated measures two-way ANOVA followed by Bonferroni post-tests to evaluate the effects of two variables (e.g., carcinogenesis and NBCn1 genotype) on the measured variable. Survival curves were compared by Gehan-Breslow-Wilcoxon test. Linear relationships (typically after log-transformation) were studied using least-squares linear regression analyses. A probability value (P-value) smaller than 0.05 was considered statistically significant; n equals number of mice. Statistical analyses were performed using GraphPad Prism 5.02 software.

Results

We investigated susceptibility of NBCn1 KO mice to breast cancer development after subcutaneous implantation of medroxyprogesterone acetate (MPA) pellets and treatment with 7,12-dimethylbenz(α)anthracene (DMBA) by gavage.

KO of NBCn1 Delays Breast Cancer Development and Inhibits Tumor Growth

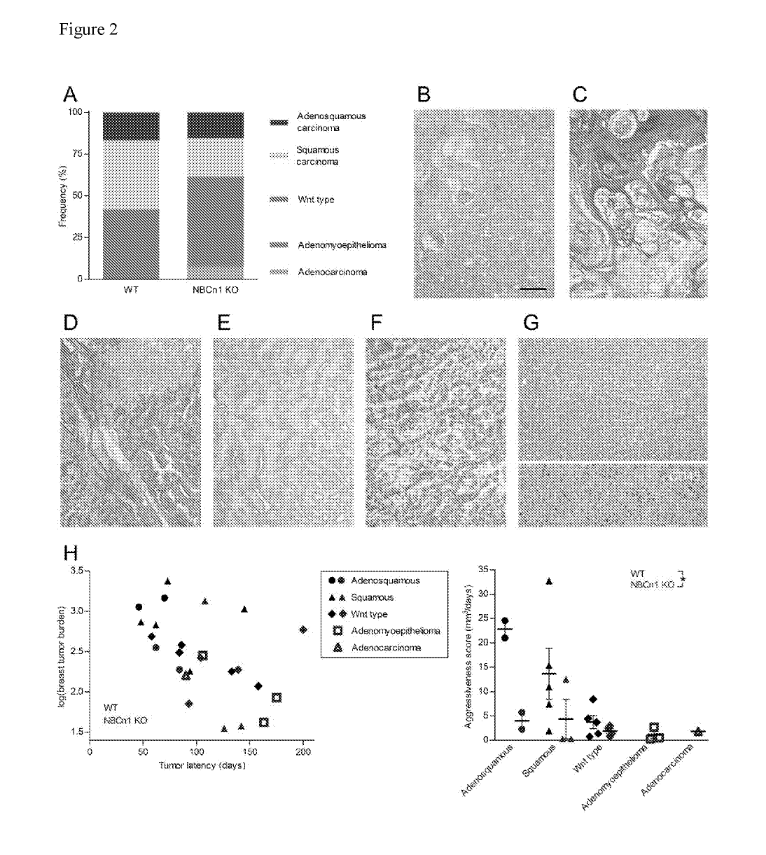

After completion of the chemical induction procedure, wild type (WT) mice developed breast cancer with an average latency of 83±8 days (FIG. 1A). In NBCn1 KO mice, tumor latency was increased to 125±9 days (FIG. 1A, P<0.01, unpaired two-tailed Student's t-test). At the end of the observation period, when the mice were 312 days old, a similar proportion of NBCn1 KO and WT mice had developed detectable breast tumors (FIG. 1A). We evaluated growth rates of breast tumors by determining tumor burden 14 days after first tumor detection (FIG. 1B). The lower detection limit by palpation is tumors 3-4 mm in diameter and is not expected to depend on NBCn1 genotype. By this approach, tumor growth rate was found ˜65% lower in NBCn1 KO than WT mice (FIG. 1B). Two mice were censored and excluded from further analysis because they developed lymphomas (FIG. 2G) before breast tumors were detectable.

Breast Tumors in NBCn1 KO Mice are Histopathologically Distinct from Tumors in WT Mice

The histopathology of breast tumors developed in NBCn1 KO mice was more diverse than in WT mice (FIG. 2A). All breast tumors detected in WT mice were squamous carcinomas (FIG. 2B), adenosquamous carcinomas (FIG. 2C) or Wnt type tumors (FIG. 2D) whereas these tumor subtypes made up around 80% of breast tumors in NBCn1 KO mice (FIG. 2A). The remaining breast tumors in NBCn1 KO mice were adenomyoepitheliomas (FIG. 2E) or adenocarcinomas (FIG. 2F). Histology and anti-CD45 immunohistochemical staining of the developed lymphomas are shown in FIG. 2G.

To determine whether the effects of NBCn1 KO on tumor development and growth correlated with histopathology, we calculated for each histopathological tumor subtype an aggressiveness score defined as the ratio between tumor burden (at day 14 after first tumor detection) and tumor latency. As shown in FIG. 2H, NBCn1 KO reduced the aggressiveness score for the three tumor types observed in both WT and NBCn1 KO mice (P<0.001, two-way ANOVA), and the additional tumor types observed only in NBCn1 KO mice had low aggressiveness scores (FIG. 2H). These findings suggest that NBCn1 KO inhibits tumor aggressiveness also after correction for histopathology.

KO of NBCn1 Inhibits Cellular Net Acid Extrusion and Causes Intracellular Acidification

We investigated the mechanisms of net acid extrusion using organoids freshly isolated from primary breast cancer and matched normal breast tissue. As shown in FIG. 3A, breast organoids were dominated by cells positive for the epithelial cell marker cytokeratin (CK)19 and showed low abundance of cells positive for the myofibroblast marker smooth muscle α-actin (α-SMA). The average pHᵢ recordings shown in FIG. 3B illustrate that cellular net acid extrusion in organoids from breast cancer tissue was largely Na⁺-dependent both in the presence (88±4% in WT and 92±3% in NBCn1 KO, n=8) and in the absence (94±4% in WT and 101±5% in NBCn1 KO, n=8-9) of CO₂/HCO₃⁻.

Intracellular intrinsic buffering capacity was increased in organoids from breast cancer compared to normal breast tissue but not systematically affected by NBCn1 KO (FIG. 3C). Based on pHᵢ recovery rates and intracellular buffering capacities, we calculated net Na⁺-dependent acid-base transport activities as functions of pHᵢ (FIG. 3D,E). Cellular net acid extrusion increased approximately linearly when pHᵢ was decreased in both normal and malignant breast tissue (FIG. 3D,E). In breast cancer tissue from WT mice, net acid extrusion was strongly CO₂/HCO₃⁻-dependent as demonstrated by high transport activities at alkaline pHᵢ (i.e., above 7.2) only in the presence of CO₂/HCO₃⁻ (FIG. 3D). This CO₂/HCO₃⁻-dependent transport activity was not observed in breast cancer tissue from NBCn1 KO mice (FIG. 3E) nor in normal breast tissue from WT or NBCn1 KO mice (FIG. 3D,E) where net acid extrusion was almost entirely CO₂/HCO₃⁻-independent. In the absence of CO₂/HCO₃⁻, net acid extrusion was very similar in normal breast tissue and breast cancer tissue whether from WT or NBCn1 KO mice (FIG. 3D,E). These findings demonstrate that NBCn1 was responsible for the upregulated net acid extrusion in the breast cancer tissue whereas no major change in Na⁺/H⁺-exchange activity occurred during breast carcinogenesis.

Steady-state pHᵢ of cancer tissue can be surprisingly high even in an acidic environment, often resulting in an inverted pH gradient (i.e., with pHᵢ>pHₒ) across the plasma membrane. Indeed, we observed that pHᵢ in breast cancer organoids from WT mice was higher than the pH (7.4) of the bath solution when investigated in the presence of CO₂/HCO₃⁻ (pHᵢ=7.62±0.09; P<0.05 vs. 7.4, one-sample t-test). Furthermore, steady-state pHᵢ in the presence of CO₂/HCO₃⁻ was lower in breast cancer tissue from NBCn1 KO than WT mice (FIG. 3F) demonstrating a critical role for NBCn1 in steady-state pHᵢ control. In accordance, whereas steady-state pHᵢ in breast cancer organoids from WT mice decreased dramatically (˜0.4 units) upon omission of CO₂/HCO₃⁻,steady-state pHᵢ in breast cancer organoids from NBCn1 KO mice was similar in the presence and absence of CO₂/HCO₃⁻ (FIG. 3F). When pHₒ was reduced to 6.8, the differences in steady-state pHᵢ between breast cancer organoids from WT and NBCn1 KO mice and their patterns of CO₂/HCO₃⁻-dependency persisted (FIG. 3F). Steady-state pHᵢ in the normal breast tissue was lower than in the breast cancer tissue (FIG. 3F) and—as expected for most normal cells—was below the pH of the bath solution (for WT: pHᵢ=7.20±0.03, for NBCn1 KO: pHᵢ=7.26±0.02,for both: P<0.001 vs. 7.4, one-sample t-tests). Also, in contrast to the CO₂/HCO₃⁻-dependency of pHᵢ in breast cancer tissue from WT mice, steady-state pHᵢ in normal breast tissue from both WT and NBCn1 KO mice was unaffected by omission of CO₂/HCO₃⁻ (FIG. 3F).

NBCn1 is Strongly Upregulated During Breast Carcinogenesis

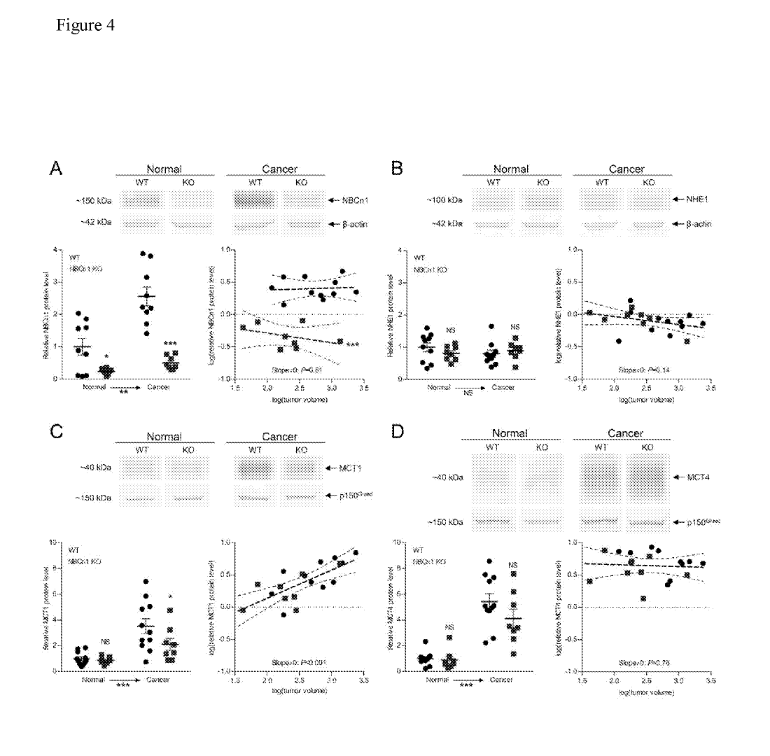

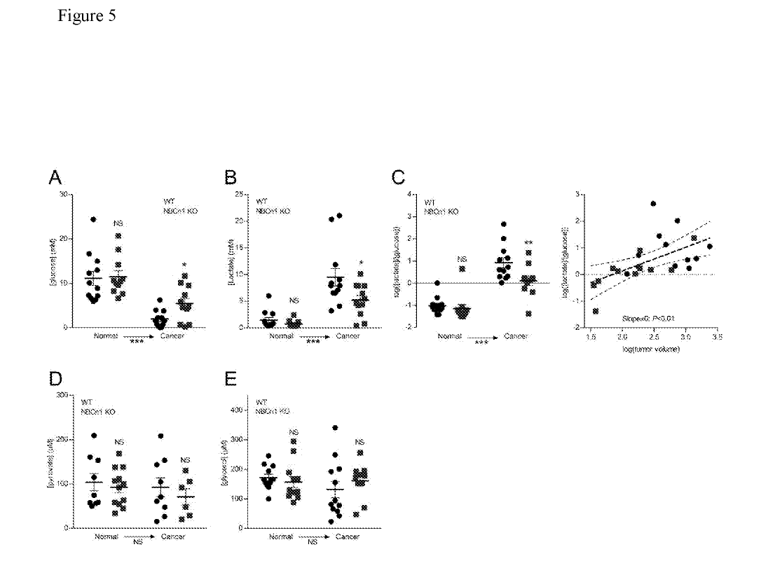

In WT mice, upregulation of Na⁺,HCO₃⁻-cotransport during breast carcinogenesis was associated with a 2.5-fold higher NBCn1 protein expression in organoids from breast cancer tissue compared to normal breast tissue (FIG. 4A, left panel). No correlation was observed between NBCn1 expression level and tumor size (FIG. 4A, right panel), suggesting that the increase in NBCn1 expression is an early event in carcinogenesis. Consistent with the lack of effect of carcinogenesis on the rate of net Na⁺-dependent acid extrusion in the absence of CO₂/HCO₃⁻ (FIG. 3D,E), protein expression of the Na⁺/H⁺-exchanger NHE1 (Slc9a1) was similar in organoids from breast cancer and normal breast tissue whether from WT or NBCn1 KO mice (FIG. 4B).

Protein expression of monocarboxylate transporters MCT1 (Slc16a1) and MCT4 (Slc16a3) was substantially upregulated during breast carcinogenesis (FIG. 4C,D) consistent with the known roles of monocarboxylate transporters in elimination of intracellular lactate generated through glycolytic metabolism. Upregulation of MCT1 expression was smaller in tumors from NBCn1 KO than WT mice (FIG. 4C, left panel) whereas expression of MCT4 was not significantly different in tumors from NBCn1 KO and WT mice (FIG. 4D, left panel). These differences between MCT1 and MCT4 expression were reflected in their relation to tumor size: while MCT1 expression increased as a function of tumor size (FIG. 4C, right panel), expression of MCT4 was tumor size independent (FIG. 4D, right panel).

Glycolytic Metabolism is Reduced in Breast Cancer of NBCn1 KO Mice

To assess tumor metabolism in vivo, we implanted microdialysis probes in breast tumors and matched normal breast tissue in the same mice. In breast tumors of WT mice, we found reduced concentrations of glucose ([glucose]) and increased concentrations of lactate ([lactate]) compared to normal breast tissue (FIG. 5A,B) consistent with accelerated glycolytic metabolism. In breast tumors of NBCn1 KO mice, [glucose] was higher and [lactate] lower than in breast tumors of WT mice (FIG. 5A,B). Combining these findings, we find that the [lactate]/[glucose] ratio is lower in breast tumors of NBCn1 KO than WT mice (FIG. 5C, left panel) supporting that glycolysis in breast tumors of NBCn1 KO mice is inhibited compared to tumors of WT mice. The [lactate]/[glucose] ratio increased as a function of increasing breast tumor size (FIG. 5C, right panel) consistent with higher flow of glucose to lactate in medium- and larger-sized breast tumors relative to small breast tumors. We observed no differences in the concentrations of pyruvate ([pyruvate]) or glycerol ([glycerol]) between breast tumors and matched normal breast tissue or between tissue from NBCn1 KO and WT mice (FIG. 5D,E).

Cell Signaling is Modified by Breast Carcinogenesis and NBCn1 KO

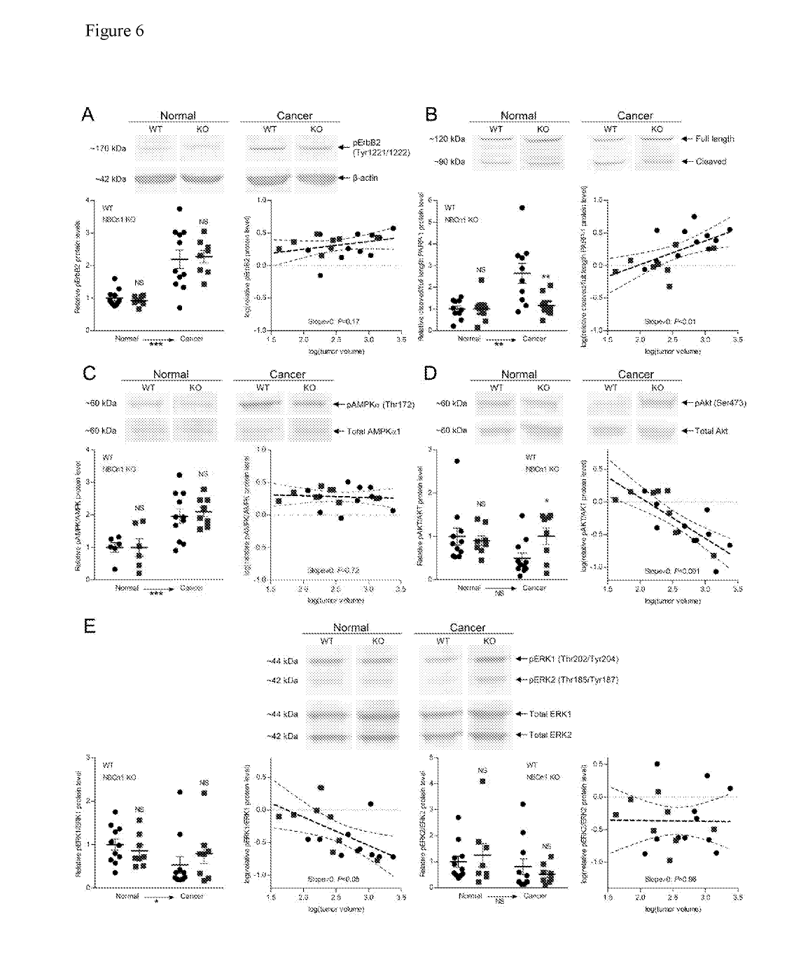

Intracellular signaling is fundamentally altered in many types of cancer. We tested here whether tumors from NBCn1 KO mice differed from tumors from WT mice in their intracellular signaling profile. Overexpression of an N-terminally truncated ErbB2 receptor has previously been shown to cause expressional upregulation of NBCn1 in cultured MCF-7 human breast cancer cells; and using isolated organoids, we find that the level of phosphorylated ErbB2 is increased in breast cancer tissue compared to matched normal breast tissue (FIG. 6A, left panel). No difference in ErbB2 phosphorylation was observed between breast tumors from NBCn1 KO and WT mice (FIG. 6A, left panel) and the level of ErbB2 phosphorylation was independent of tumor size (FIG. 6A, right panel).

The cleaved fraction of poly ADP-ribose polymerase (PARP)-1 is a measure of programmed cell death. Consistent with increased apoptosis in the harsh tumor microenvironment, it was found that proteolytic PARP-1 cleavage was increased in breast cancer tissue compared to normal breast tissue (FIG. 6B, left panel). The cleaved fraction of PARP-1 was lower in tumors from NBCn1 KO than WT mice (FIG. 6B, left panel) and increased as a function of tumor size (FIG. 6B, right panel). AMPK, AKT and Erk1/2 signaling is prominent in many types of cancer where it is thought to link energy sensing to e.g., cell metabolism, proliferation and cell death. AMPK phosphorylation was increased in breast tumor tissue compared to normal breast tissue but did not differ between NBCn1 KO and WT mice (FIG. 6C, left panel). No correlation between the level of AMPK phosphorylation and tumor size was observed (FIG. 6C, right panel). AKT phosphorylation was reduced in breast tumors from WT mice compared to normal breast tissue (FIG. 6D, left panel). This reduction in AKT phosphorylation did not occur during breast carcinogenesis in NBCn1 KO mice (FIG. 6D, left panel), however, when AKT phosphorylation was plotted as a function of tumor volume, a significant negative correlation between tumor size and AKT phosphorylation was observed independently of NBCn1 genotype (FIG. 6D, right panel). In WT mice, ERK1 phosphorylation was decreased in breast cancer tissue compared to normal breast tissue, whereas no significant change in ERK2 phosphorylation was seen (FIG. 6E). The level of ERK1 phosphorylation in the breast cancer tissue decreased when tumor size increased independently of NBCn1 genotype (FIG. 6E, second panel). No correlation between tumor volume and ERK2 phosphorylation was seen (FIG. 6E, fourth panel).

Cell Proliferation is Reduced in Breast Cancer of NBCn1 KO Compared to WT Mice

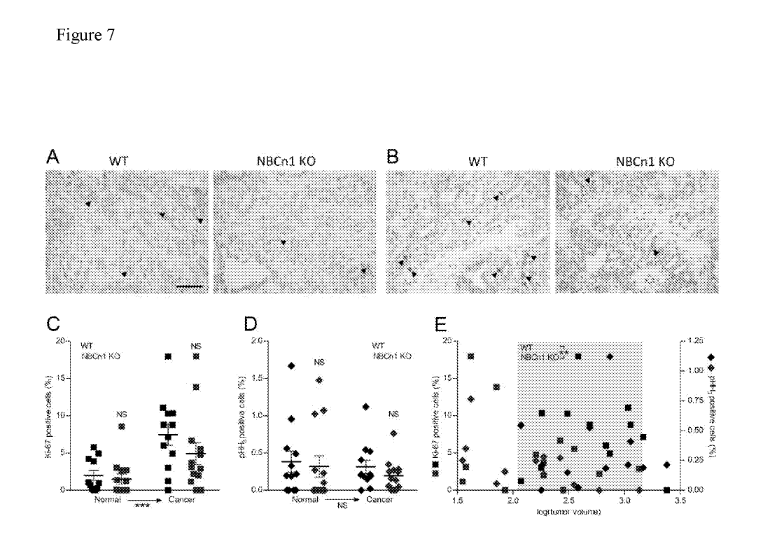

To assess the proliferative activity in breast cancer and normal breast tissue, we performed immunohistochemical staining for Ki-67 and Ser10 phosphorylated histone H₃ (pHH₃). Representative images are shown in FIGS. 7A (Ki-67) and 7B (pHH₃). The fraction of cells positive for Ki-67 increased substantially during breast carcinogenesis (FIG. 7C) whereas the fraction of cells positive for pHH₃ was similar in breast cancer and normal breast tissue (FIG. 7D). In the smallest breast tumors (<100 mm³) from NBCn1 KO mice, the Ki-67 and pHH₃ labeling indices were highly variable, whereas they were consistently low in medium- to large-sized breast tumors (FIG. 7E). Due to tumor size heterogeneity between WT and NBCn1 KO mice, the overall Ki-67 and pHH₃ labeling indices did not differ between breast cancer tissue from NBCn1 KO and WT mice (FIGS. 7C and D). However, in the range of tumor sizes covered by both genotypes (i.e., in the size interval between the smallest breast tumor from WT mice and the largest breast tumor from NBCn1 KO mice; shaded area in FIG. 7E, n=8-10), the fraction of cells positive for Ki-67 (3.5±0.8% in tumors from NBCn1 KO compared to 8.2±1.5% in tumors from WT mice) and pHH₃ (0.12±0.05% in tumors from NBCn1 KO compared to 0.36±0.11% in tumors from WT mice) was reduced by approximately 60% (FIG. 7E).

Taken together, the data demonstrate that NBCn1 KO inhibits cell proliferation when tumor size is taken into account (FIG. 7E). Since the effect of NBCn1 KO is most noticeable in medium- to large-sized breast tumors (FIG. 7E), which have higher glycolytic activities (FIG. 5C, right panel) and consequent higher rates of intracellular acid loading, our data are consistent with the interpretation that NBCn1 is required for neutralizing acidic waste products from tumor metabolism.

Discussion

The present example demonstrates a causal link between NBCn1 and breast cancer development and growth. The data show that disruption of NBCn1 expression results in a 50% longer tumor latency period and a 65% reduction in breast cancer growth rate. In breast cancer tissue from NBCn1 KO mice, CO₂/HCO₃⁻-dependent net acid extrusion is abolished (FIG. 3D,E), steady-state pHᵢ is lower (FIG. 3F), glycolytic activity is inhibited (FIG. 5) and cell proliferation is reduced (FIG. 7) compared to breast cancer tissue from WT mice. Although histopathologically different from typical human breast carcinomas (FIG. 2), the breast cancer tissue in the investigated murine model accurately resembles human breast cancer tissue in terms of acid-base transporter expression, Na⁺,HCO₃⁻-cotransport dependency and steady-state pHᵢ regulation.

These results illustrate the potential of inhibiting breast cancer development by targeting cancer cell metabolism and/or acidic waste product elimination. Previously, the Na⁺/H⁺exchanger NHE1 has been mentioned in this connection. NHE1 is important for pHᵢ regulation in many cancer types (Loo S Y, Chang M K, Chua C S, Kumar A P, Pervaiz S, Clement M V. NHE-1: a promising target for novel anti-cancer therapeutics. Curr. Pharm. Des 2012; 18:1372-1382), but does not play a predominant role for overall pHᵢ regulation in the current murine breast cancer model (FIG. 3) or in human breast cancer biopsies. In human and murine (FIG. 3) breast cancer tissue, Na⁺/H⁺-exchange activity apparently contributes to global pHᵢ regulation mainly at very acidic pHᵢ levels. At neutral pHᵢ conditions, NHE1 may still play a role for pH control in local restricted intracellular or extracellular spaces or have transport-independent effects such as cytoskeletal anchoring.

By plotting protein expression levels and metabolic values as functions of tumor volume (FIG. 4-7), we take into account that functional and expressional changes may occur during breast cancer progression. Due to the relation between breast cancer histopathology and tumor growth rate (FIG. 2H), tumor volume becomes in part a surrogate measure for histopathology. Nevertheless, a distinct pattern arises where expression of some proteins (e.g., NHE1 and pERK2) is unaltered in cancer compared to normal tissue, whereas the expression of other proteins changes during breast carcinogenesis and is either unrelated (e.g., NBCn1, MCT-4, pErbB2 and pAMPK), positively related (MCT-1 and cleaved PARP-1) or negatively related (pAKT and pERK1) to tumor volume; FIGS. 4 and 6). These findings imply that while expression of some proteins (e.g., NBCn1) changes early in carcinogenesis, the change in expression of other proteins occurs later when the tumors start expanding. The upregulation of NBCn1 early in carcinogenesis, however, supports its essential role in breast cancer development.

NBCn1 expression in cultured MCF-7 breast cancer cells can be upregulated by enhanced signaling downstream of ErbB2 receptors. This mechanism may also contribute to NBCn1 upregulation in the current model of breast carcinogenesis because ErbB2 phosphorylation was increased in breast cancer compared to normal breast tissue (FIG. 6A). Consistent with this notion, the levels of ErbB2 phosphorylation and NBCn1 expression were both independent of tumor volume (FIGS. 4A and 6A, right panels). Still, the pronounced effect of NBCn1 inhibition across multiple histopathological subtypes (FIG. 2) supports a general importance of NBCn1 in breast carcinogenesis as described for human breast cancer tissue where upregulated NBCn1 expression and Na⁺,HCO₃⁻-cotransport activity are not restricted to tumors with particular receptor (i.e., HER2/ErbB2 or estrogen) expression profiles. Further supporting that Na⁺,HCO₃⁻-cotransport is important across multiple cancer types and conditions, a recent study found comparable levels of Na⁺-dependent HCO₃⁻-transport in multiple cultured cancer cell lines irrespective of oxygenation status (Hulikova A, Harris A L, Vaughan-Jones R D, Swietach P. Regulation of intracellular pH in cancer cell lines under normoxia and hypoxia. J Cell Physiol 2013; 228:743-752.).

Tumor cell proliferative activity was assessed by immunohistochemical staining for Ki-67 and pHH₃ (FIG. 7). The Ki-67 and pHH₃ labeling indices correlate inversely with prognosis for human cancer patients. Thus, the lower Ki-67 and pHH₃ labeling indices in tumors from NBCn1 KO mice support the implications of NBCn1 inhibition for breast cancer development and progression. It is well-accepted that glycolytic conversion of glucose to lactic acid increases with tumor size (FIG. 5). Based on the effect of NBCn1 KO on proliferation particularly in medium- to larger-sized tumors (FIG. 7E), it appears that NBCn1 could permit rapid tumor growth by facilitating acidic waste product elimination in tumors relying on high glycolytic activity.

The difference in histopathology between breast tumors from WT and NBCn1 KO mice is intriguing. The slower cancer development and growth (FIG. 1) and lower proliferative activity (FIG. 7E) in NBCn1 KO mice could provide time for less aggressive tumor types to develop. These less aggressive tumor types (e.g., adenomyoepitheliomas) would typically not be seen in WT mice because earlier development of more aggressive tumor types results in early euthanasia.

In conclusion, the present example shows that NBCn1 is causally related to breast cancer development. Genetic disruption of NBCn1 prolongs breast tumor latency, slows tumor growth and inhibits tumor cell proliferation. In congruence, NBCn1 mediates the upregulated cellular net acid extrusion in breast cancer tissue and maintains the alkaline steady-state pHᵢ and inverted pH gradient across the plasma membrane. Also, compared to breast tumors in WT mice, the metabolic profile and protein expression pattern of breast tumors in NBCn1 KO mice are more similar to those of normal breast tissue. These findings establish NBCn1 as a target for anti-cancer therapy.

### Example 2

Na⁺,HCO₃⁻-cotransporter NBCn1 increases pHᵢ gradients, filopodia and migration of smooth muscle cells and promotes arterial remodeling

Abstract

Aims: Arterial remodeling can cause luminal narrowing and obstruct blood flow. We tested the hypothesis that cellular acid-base transport mediated by NBCn1 facilitates proliferation and migration of vascular smooth muscle cells (VSMCs) and enhances remodeling of conduit arteries.

Methods and Results: Na⁺,HCO₃⁻-cotransport via NBCn1 (Slc4a7) mediates net acid extrusion and controls steady-state intracellular pH (pHᵢ) in VSMCs of mouse carotid arteries and primary aortic explants. Carotid arteries undergo hypertrophic inward remodeling in response to partial or complete ligation in vivo but the increase in media area and thickness and reduction in lumen diameter are attenuated in arteries from NBCn1 knockout compared to wild-type mice. Wth CO₂/HCO₃⁻ present, gradients for pHᵢ (˜0.2 units magnitude) exist along the axis of VSMC migration in primary explants from wild-type but not NBCn1 knockout mice. Knockout or pharmacological inhibition of NBCn1 also reduces filopodia and lowers initial rates of VSMC migration after scratch-wound infliction. Interventions to reduce H⁺-buffer mobility (omission of CO₂/HCO₃⁻ or inhibition of carbonic anhydrases) reestablish axial pHᵢ gradients, filopodia, and migration rates in explants from NBCn1 knockout mice. Omission of CO₂/HCO₃⁻ also lowers global pHᵢ and inhibits proliferation in primary explants.

Conclusions: Under physiological conditions (i.e., with CO₂/HCO₃⁻ present), NBCn1-mediated HCO₃⁻ uptake raises VSMC pHᵢ and promotes filopodia, VSMC migration, and hypertrophic inward remodeling. Probabaly axial pHᵢ gradients enhance VSMC migration whereas global acidification inhibits VSMC proliferation and media hypertrophy after carotid artery ligation. These findings support a key role of NBCn1 mediated acid-base transport in development of occlusive artery disease.

Background

Occlusive artery disease is a leading cause of human morbidity and mortality. Remodeling of conduit arteries typically occurs in response to altered hemodynamic forces. Although inward remodeling may serve important homeostatic functions, it can limit blood flow and contribute to ischemia.

Structural remodeling of arteries requires cell migration and possibly hyperplasia or hypertrophy. Ion transport across the plasma membrane modifies cell migration, proliferation, and growth. In addition to cell volume—which is controlled by multiple ion transport mechanisms—acid-base transporters regulate intracellular pH (pHᵢ) and local extracellular pH (pHₒ). Recent studies suggest that local pH can change cell-matrix interactions (e.g., due to modifications of integrin binding), extracellular matrix degradation (e.g., by activation of matrix metalloproteinases), cytoskeletal dynamics (e.g., through cofilin-dependent changes in actin polymerization), and focal adhesion remodeling (e.g., due to changes in talin-actin interactions). As a result, local acid-base status may fundamentally modify tissue structure.

Investigations based on cultured non-vascular cells support that the ubiquitous Na⁺/H⁺-exchanger NHE1 (Slc9a1) promotes cell division and migration. NHE1-dependent pHₒ and pHᵢ gradients along the axis of migration and predominant NHE1 expression at the leading edge of motile cells have been demonstrated. Studies of pulmonary and systemic resistance arteries and vascular smooth muscle cells (VSMCs) support the relevance of these findings for structural development and remodeling of the vascular wall. Yet, because effects of NHE1 on cell migration and cytoskeletal arrangement are at least partly independent of ion translocation, it remains controversial to what extent NHE1 modifies tissue structure via changes in pH. It should also be noted that most previous studies proposing a predominant role of NHE1 for subcellular pH regulation and cell migration were performed in absence of CO₂/HCO₃⁻ where effects of Na⁺/H⁺-exchange will likely be overestimated.

In the wall of mouse mesenteric, coronary, and cerebral resistance arteries, omission of CO₂/HCO₃⁻ has dramatic effects on pHᵢ. With CO₂/HCO₃⁻ present, Na⁺,HCO₃⁻-cotransport mediated via NBCn1 (Slc4a7) dominates net acid extrusion in the near-neutral pHᵢ range, whereas Na⁺/H⁺-exchange activity is important for acid extrusion primarily during pronounced intracellular acidification. Molecular mechanisms of global pHᵢ regulation have been studied in detail for VSMCs, but mechanisms of pH regulation in subcellular compartments and local restricted spaces remain to be investigated. Although there is evidence for NBCn1 expression in rat and mouse aortas, the significance of NBCn1 for conduit artery function and structure is unresolved. NHE1 and NBCn1 mediate corresponding acid-base transport functions when the CO₂/HCO₃⁻-buffer is equilibrated but it is still unclear whether they share activation patterns, play comparable roles for local pH regulation, and contribute similarly to cell signaling. Other than providing substrate for acid-base transporters, CO₂ and HCO₃⁻ are important intracellular and extracellular buffers that increase the apparent H⁺ mobility. It is plausible that CO₂/HCO₃⁻-dependent processes modify relative acid-base transport activities at different subcellular locations (e.g., at the leading compared to rear end of migrating cells) and additionally contribute to dissipating pH gradients by facilitating diffusion of acid-base equivalents within the intra- and extracellular compartments. In the current study, we tested the hypothesis that local pHᵢ in VSMCs is controlled by the interplay between NBCn1-mediated Na⁺,HCO₃⁻-cotransport and mobile H⁺ buffers to coordinate and promote cellular migration and proliferation that are key processes in conduit artery remodeling.

Methods

NBCn1 knockout (KO) mice were generated and maintained on the 129/SvJ genetic background. The experiments conformed to guidelines from Directive 2010/63/EU of the European Parliament on protection of animals used for scientific purposes and were approved by the Danish Animal Experiments Inspectorate (2012-15-2934-00286).

In Vitro Analyses

Primary explants were produced from thoracic aortas and common carotid arteries of NBCn1 KO and wild-type (WT) mice. To best maintain the cell morphology and phenotype of native VSMCs, cultured cells were never passaged but investigated directly in primary explants.

Using wide-field fluorescence microscopy, global pHᵢ was recorded in arteries and primary explants loaded with the pH-sensitive fluorophore BCECF. Recordings of pHᵢ in subcellular domains were performed by confocal fluorescence microscopy of cells loaded with the pH-sensitive fluorophore carboxy SNARF-1. Intracellular buffering capacities were calculated from changes in pHᵢ upon washout of NH₄Cl. No difference in total intracellular buffering capacity (β) was seen between primary explants from NBCn1 KO and WT mice (β_{WT}=15±2 mM, β_{KO}=17±2 mM, n=8; P=0.45, unpaired two-tailed Student's t-test) evaluated at pHᵢ 6.65-6.80.

Expression of transcripts coding for Na⁺,HCO₃⁻-cotransporters was investigated by reverse transcription and polymerase chain reaction. Experiments without addition of reverse transcriptase were performed to control for genomic amplification.

Rates of wound healing in primary explants were calculated from time-lapse images captured at 5-minute intervals after scratch infliction. Experiments were performed in Minimum Essential Medium with 10% fetal calf serum or—to avoid binding of the non-selective Na⁺,HCO₃⁻-cotransport inhibitor S0859 to medium or serum components—in physiological saline solutions (PSS). Cell size was estimated based on cell density in confluent areas. Total filopodia length was determined relative to the length of the migrating front (i.e., twice the scratch length). Cell proliferation was evaluated (a) by counting the relative number of cells dividing during six hours time-lapse imaging and (b) as the proportion of cells positive for the thymidine analogue bromodeoxyuridine (BrdU) after six hours incubation.

The PSS was composed of (in mM) 134 Na⁺, 4 K⁺, 1.6 Ca²⁺, 1.2 Mg²⁺, 111 Cl⁻, 24 HCO₃⁻, 1.2 SO₄²⁻, 1.18 H₂PO₄⁻, 10 HEPES, 5.5 glucose, 0.03 EDTA. In Na⁺-free solutions, Na⁺ was substituted with an equimolar amount of N-methyl-D-glucammonium (NMDG⁺), except for NaHCO₃, which was replaced with choline-HCO₃. In HCO₃⁻-free solutions, HCO₃⁻ was replaced with an equimolar amount of Cl⁻. HCO₃⁻-containing solutions were aerated with a gas mixture of 5% CO₂/balance air, whereas HCO₃⁻-free solutions were gassed with air (i.e., 21% O₂/balance N₂). All solutions were titrated to pH 7.4 at 37° C.

In Vivo Experiments

NBCn1 KO and WT mice were anaesthetized with isoflurane and the right common carotid artery dissected free of surrounding tissue. A ligature was placed around the common carotid artery near the bifurcation (complete ligation) or around the internal and external carotid arteries above the occipital artery but below the superior thyroid artery (partial ligation). In sham-operated animals, the carotid artery was surgically isolated but no ligature was placed. The degree of remodeling was analyzed four weeks after the surgical procedure.

Statistics

Data are given as mean±SEM; n equals number of mice. P<0.05 was considered statistically significant. Single variables were compared between two groups by paired or unpaired two-tailed Student's t-test and between more than two groups by one-way ANOVA followed by Bonferroni post-tests. Regular or repeated measures two-way ANOVA was performed to compare effects of two variables on a third variable and followed by Bonferroni post-tests. Statistical tests were performed using Microsoft Excel 2010 or GraphPad Prism 5.02 software.

Results

Global pHᵢ and Slc4 transcripts in Conduit Arteries

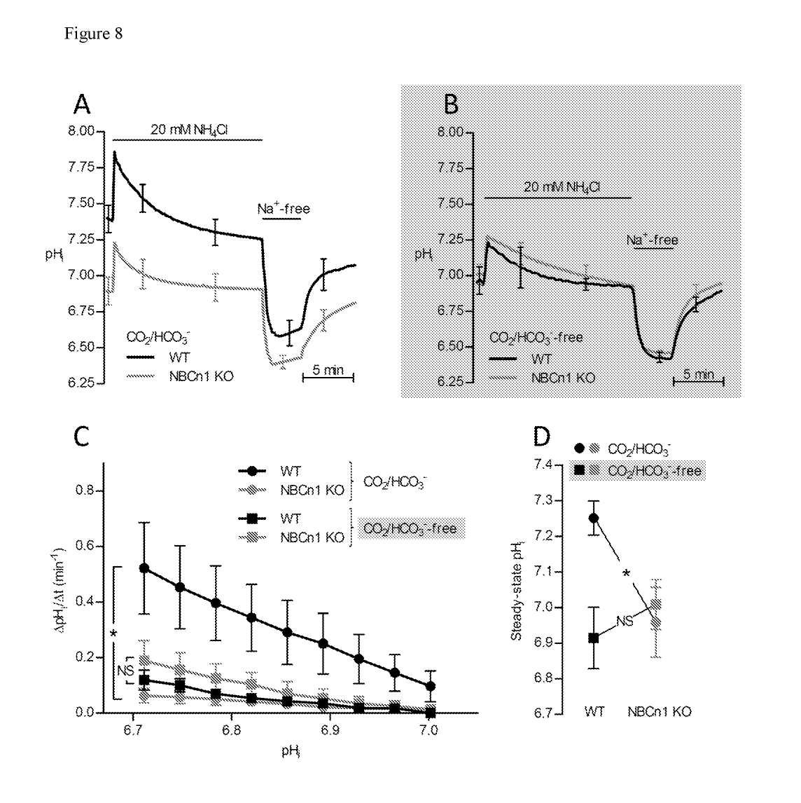

We evaluated pHᵢ regulatory function after intracellular acid loading with the NH₄⁺-prepulse technique. Washout of NH₄Cl caused intracellular acidification followed by gradual recovery towards resting pHᵢ levels (FIG. 8A,B). Acid-base transporters are regulated by pH and to allow for meaningful comparison of transporter activities under different experimental conditions, we plotted pHᵢ recovery rates as function of pHᵢ (FIG. 8C). Na⁺,HCO₃⁻-cotransport activity was prominent in VSMCs of carotid arteries from WT mice: the pHᵢ recovery rate from intracellular acidification increased as function of decreasing pHᵢ and was higher in presence than absence of CO₂/HCO₃ ⁻ when compared at equivalent pHᵢ levels (FIG. 8A-C).All experiments were performed at pHₒ 7.4. The critical role of Na⁺,HCO₃⁻-cotransport for pHᵢ regulation in VSMCs of mouse conduit arteries is consistent with previous results from rat and mouse resistance arteries.

As shown in Supplemental FIG. 8, we detected mRNA transcripts for the Na⁺,HCO₃⁻-cotransporters NBCn1, NBCe1 (Slc4a4), NDCBE (Slc4a8) and BTR1 (Slc4a11, weak bands) in segments of aorta, carotid and mesenteric arteries of 129/SvJ WT mice. NBCn1 mRNA transcripts were absent in arteries from NBCn1 KO mice whereas expression of the other Slc4-family transporters appeared unaltered or increased (FIG. 16).

Despite expression of transcripts for multiple Na⁺,HCO₃⁻-cotransporters, NBCn1 functionally predominated net acid extrusion during intracellular acidification as evidenced by the abolished Na⁺,HCO₃⁻-cotransport activity in carotid arteries from NBCn1 KO mice (FIG. 8A-C). Omission of CO₂/HCO₃⁻ strongly attenuated the Na⁺-dependent pHᵢ recovery rate from intracellular acidification in carotid arteries from WT mice but had no effect on the pHᵢ recovery rate in carotid arteries from NBCn1 KO mice (FIG. 8A-C) even at low pHᵢ where the contribution from CO₂/HCO₃⁻ to intracellular buffering is minor.

Steady-state pHᵢ in carotid arteries depended on NBCn1-mediated HCO₃⁻-transport (FIG. 8D): in presence of CO₂/HCO₃⁻ at pHₒ 7.4, steady-state pHᵢ of VSMCs was ˜0.3 lower in arteries from NBCn1 KO than WT mice (FIG. 8A,D). In the absence of CO₂/HCO₃⁻—with pHₒ maintained at 7.4—we observed no difference in steady-state pHᵢ between carotid arteries from NBCn1 KO and WT mice (FIG. 8B,D).

Taken together, these findings show that NBCn1 is responsible for the Na⁺,HCO₃⁻-cotransport and required for normal pHᵢ regulation in VSMCs of carotid arteries.

Remodeling of Carotid Arteries

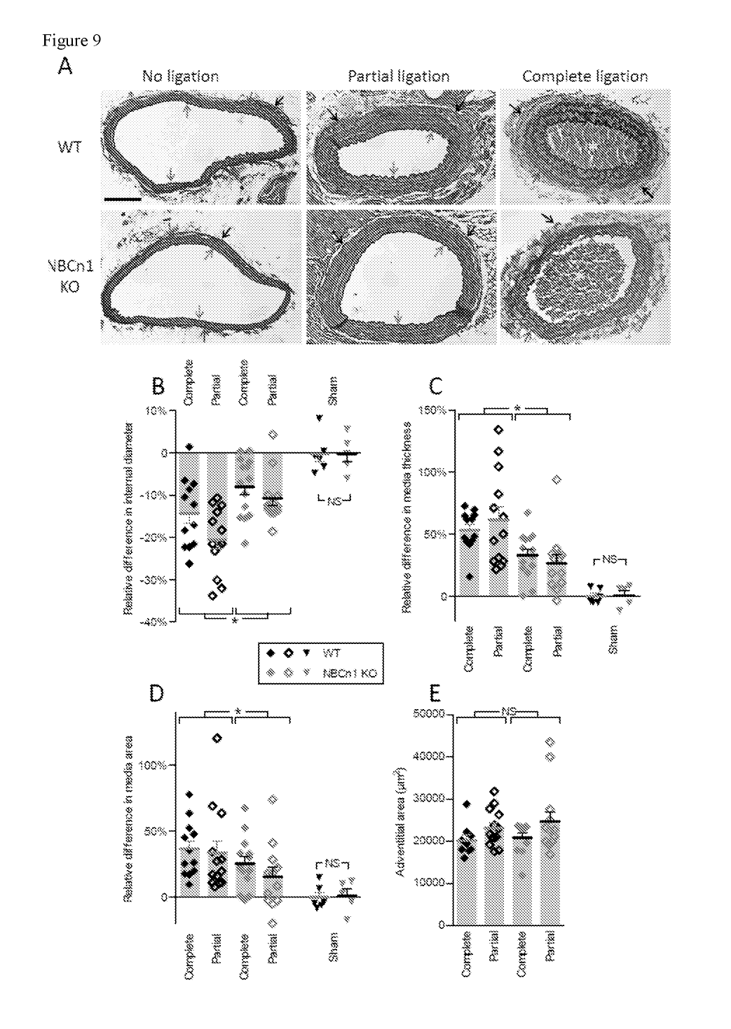

We studied morphological changes of carotid arteries in response to altered shear stress in vivo following complete or partial unilateral ligation. Compared to the complete ligation model—where carotid blood flow is completely obstructed—the partial ligation model employed here results in ˜90% reduction in carotid blood flow and limits thrombosis and endothelial cell denudation. In 129/SvJ WT mice, significant hypertrophic inward remodeling was observed after ligation (FIG. 9). Representative images are shown in FIG. 9A. The reduction in lumen diameter (FIG. 9B) and increase in media thickness (FIG. 9C) and area (FIG. 9D) were all attenuated in carotid arteries from NBCn1 KO compared to WT mice. Whereas non-ligated arteries had very little adherent adventitia, artery ligation caused significant adventitial thickening (FIG. 9A). The adventitial cross-sectional area was larger after partial than complete ligation but did not differ between arteries from NBCn1 KO and WT mice (FIG. 9E). Thus, the consequences of NBCn1 KO for arterial structure were confined to the media. The remodeling responses observed after complete or partial ligation were equivalent in nature (FIG. 9). These findings demonstrate that NBCn1 plays a central role for structural adaptations in the arterial wall in response to hemodynamic disturbances under in vivo conditions.

Global pHᵢ and Slc4 Transcripts in Primary VSMCs

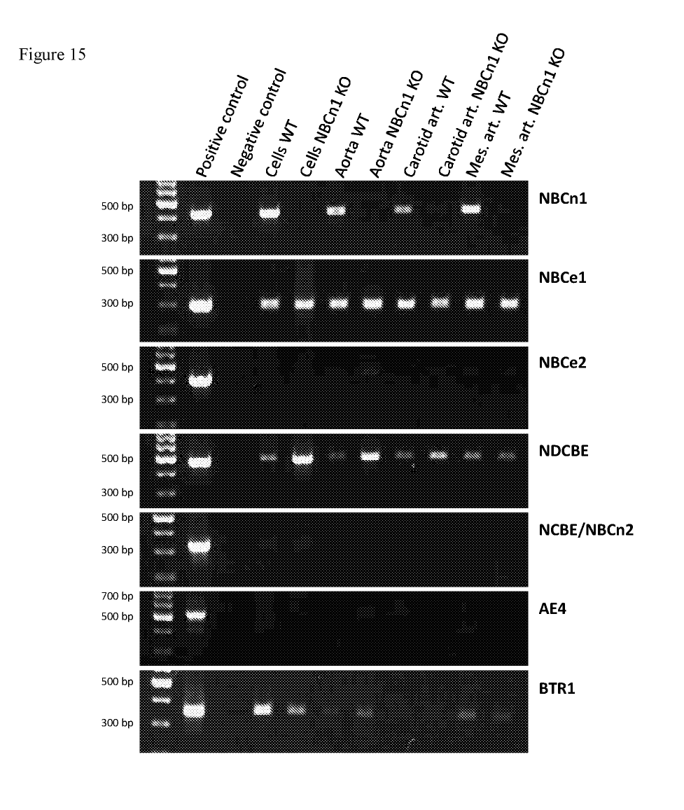

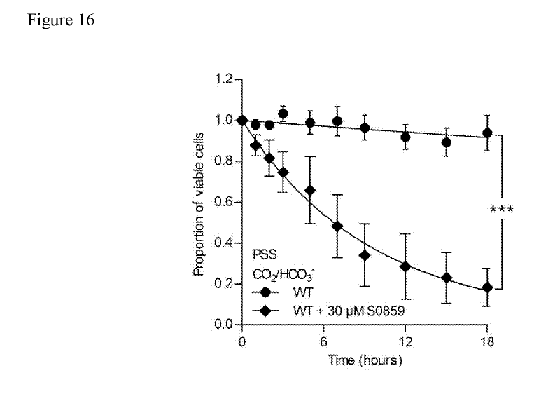

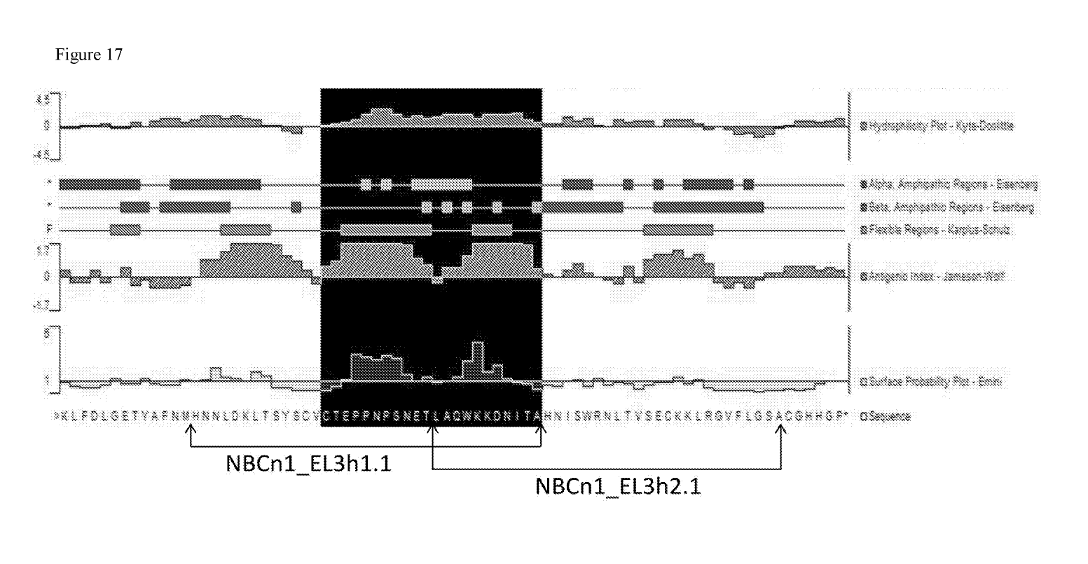

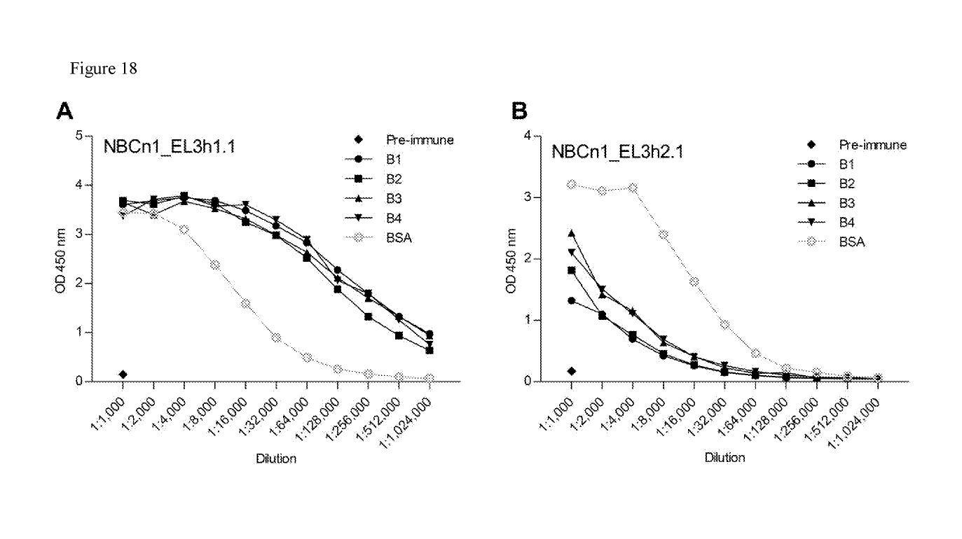

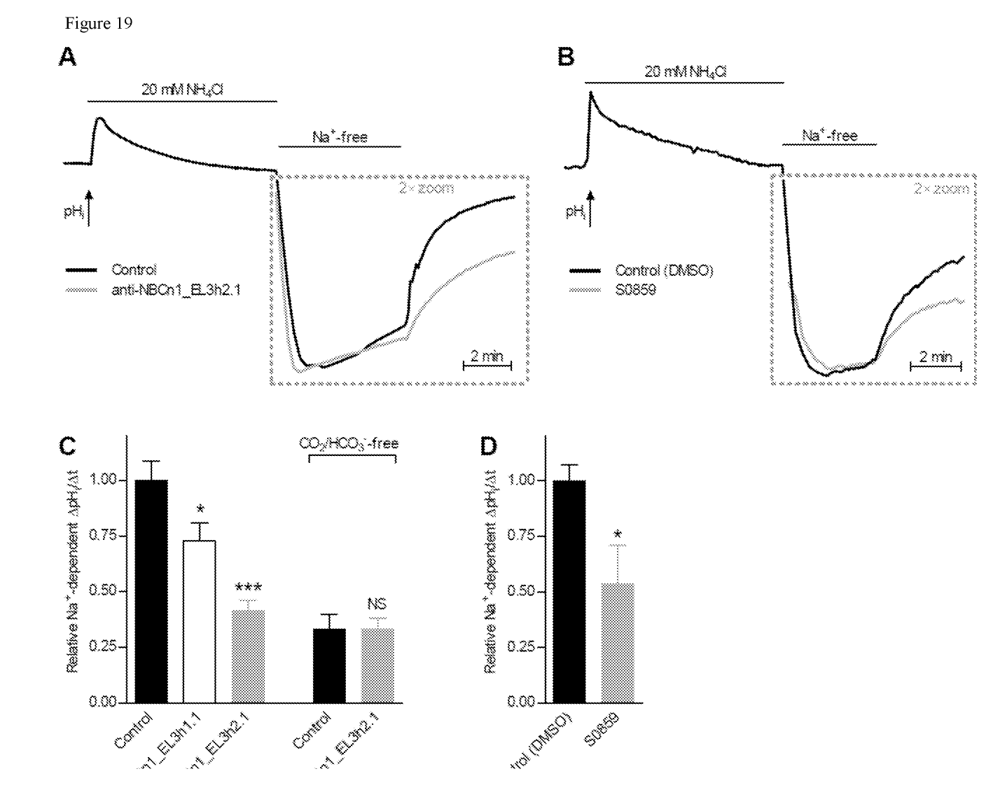

To study the contribution of NBCn1 to cellular proliferation and migration, we next produced primary explants of VSMCs from aortas of NBCn1 KO and WT mice. We reduced phenotypic effects of cell culture by performing all subsequent analyses directly on primary explants without subcultivation. The mRNA expression profile for Na⁺,HCO₃⁻-cotransporters in primary explants from NBCn1 KO and WT mice largely resembled the profile in isolated arteries (FIG. 15).

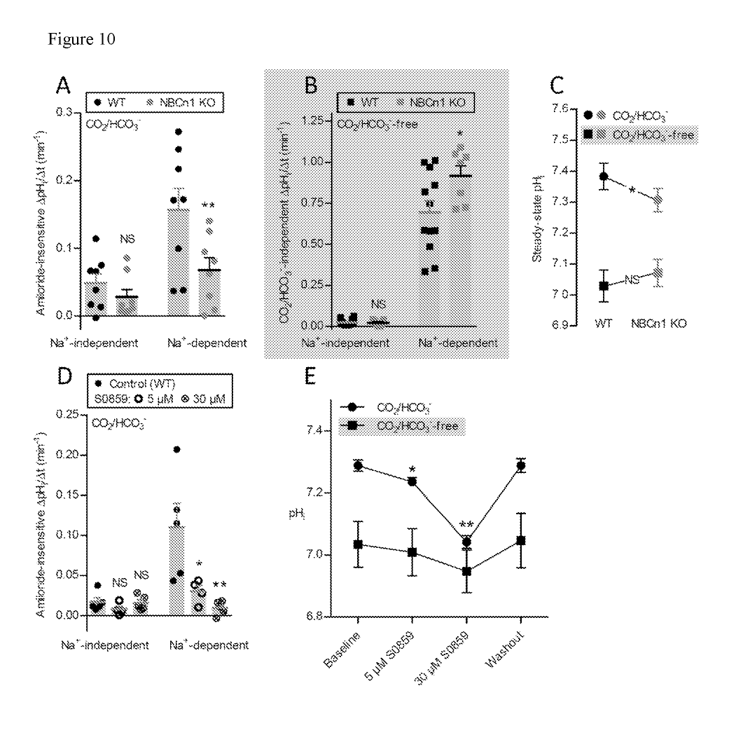

Na⁺,HCO₃⁻-cotransport activity—evaluated as the amiloride-insensitive, Na⁺-dependent pHᵢ recovery rate—contributed substantially to pHᵢ regulation in primary explants from WT mice (FIG. 10A). In explants from NBCn1 KO mice, Na⁺,HCO₃⁻-cotransport activity was ˜60% lower (FIG. 10A). Consistent with our previous findings from mesenteric arteries, Na⁺/H⁺-exchange activity—evaluated as the Na⁺-dependent, CO₂/HCO₃⁻-independent pHᵢ recovery rate—was ˜30% higher in explants from NBCn1 KO compared to WT mice (FIG. 10B). In mesenteric arteries, the increase in Na⁺/H⁺-exchange activity occurs without changes in NHE1 expression.

Global steady-state pHᵢ was only marginally reduced (by less than 0.1) in explants from NBCn1 KO compared to WT mice, reaching statistical significance only in paired tests where experiments were matched by experimental day (FIG. 10C). In contrast, omission of CO₂/HCO₃⁻ reduced steady-state pHᵢ substantially in explants from both NBCn1 KO and WT mice (FIG. 10C).

We previously showed that the non-selective Na⁺,HCO₃⁻-cotransport inhibitor S0859 does not affect cellular net acid extrusion in isolated arteries. Because S0859 inhibits Na⁺,HCO₃⁻-cotransport in cultures of MCF7 breast cancer cells, has no effect in breast cancer slices or freshly isolated breast cancer organoids, and shows unusually strong protein binding, we concluded that the inability of S0859 to inhibit Na⁺,HCO₃⁻-cotransport in arteries is explained by poor penetration into tissue preparations.Consistent with this interpretation, we show here that addition of S0859 concentration-dependently attenuates Na⁺,HCO₃⁻-cotransport activity (FIG. 10D) and steady-state pHᵢ (FIG. 10E) in primary aortic explants. The effect of 5 μM S0859 on global Na⁺,HCO₃⁻-cotransport activity (FIG. 10D) and steady-state pHᵢ (FIG. 10E) was roughly comparable to the difference between explants from WT and NBCn1 KO mice (FIG. 10A,C).

Together these findings demonstrate that NBCn1 plays a prominent—although not exclusive—role for Na⁺,HCO₃⁻-cotransport in primary explants and that Na⁺/H⁺-exchange activity is upregulated in explants from NBCn1 KO mice.

Wound Healing

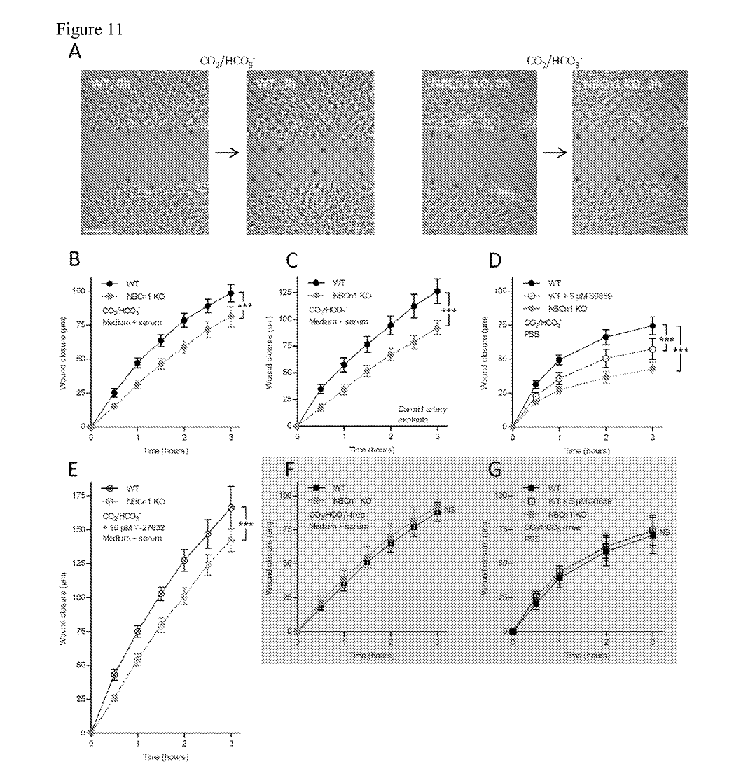

We studied VSMC migration in primary aortic and carotid explants after scratch infliction. Time-lapse movies were made to show the wound healing process and representative images of the wound healing process are shown in FIG. 11A. VSMCs from NBCn1 KO mice healed scratch-wounds slower than VSMCs from WT mice when studied in CO₂/HCO₃⁻-containing serum-supplemented medium (FIG. 4B,C) or PSS (FIG. 11D). The initial rate of wound healing was reduced by 40-60% in explants from NBCn1 KO compared to WT mice when investigated in presence of CO₂/HCO₃⁻ (FIG. 11B-D), whereas no effect of NBCn1 KO was observed on the rate of wound healing in absence of CO₂/HCO₃⁻ (FIG. 11F,G). Here and in subsequent experiments, pHₒ was kept constant at 7.4 when switching between different buffer solutions. Application of 5 μM S0859 inhibited wound healing in the presence of CO₂/HCO₃⁻ (FIG. 11D) but not in its nominal absence (FIG. 11G). These findings demonstrate a key role of Na⁺,HCO₃⁻-cotransport via NBCn1 for wound healing.

We have previously shown that KO of NBCn1 inhibits rho-kinase-dependent signaling in VSMCs of mouse resistance arteries. A number of studies have suggested that rho-kinase activity enhances cell migration, although there are also studies showing an inverse relationship between rho-kinase activity and cell migration. In primary aortic explants, application of 10 μM rho-kinase inhibitor Y-27632 stimulated wound healing without affecting the relative difference between NBCn1 KO and WT mice (FIG. 11E) supporting that altered rho-kinase activity does not explain the slower rate of wound healing in explants from NBCn1 KO mice.

Proliferation of Isolated VSMCs

In addition to altered migration, a change in proliferative activity can alter the rate of wound healing. We investigated proliferation of VSMCs based on (a) the number of cell divisions observed in time-lapse recordings and (b) incorporation of thymidine-analogue BrdU.

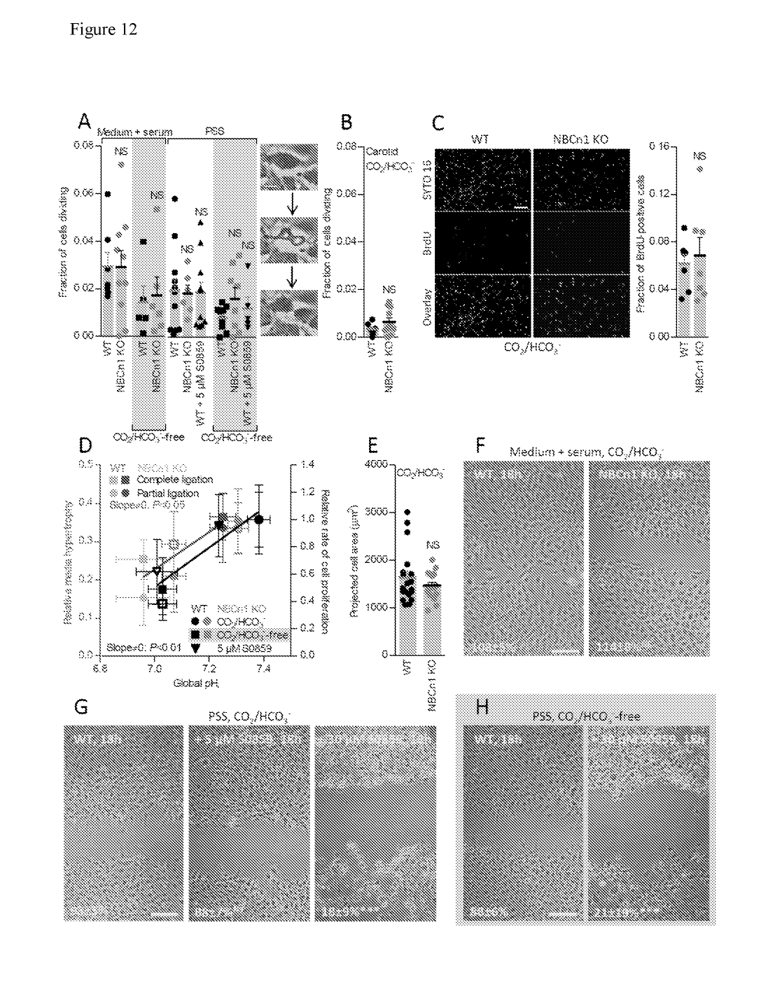

Based on time-lapse imaging, we found a similar proportion of VSMCs from NBCn1 KO and WT mice undergoing mitosis during six hours observation in serum-containing culture medium (FIG. 12A,B). Although the relative number of mitoses was reduced when VSMCs were investigated in PSS, we still saw no effect of NBCn1 KO or 5 μM S0859 under these conditions (FIG. 12A). The unaltered proliferative activity of VSMCs from NBCn1 KO mice in vitro was confirmed by BrdU incorporation experiments: the relative number of cells containing recently synthesized DNA material after six hours incubation was not significantly different between explants from NBCn1 KO and WT mice (FIG. 12C). As expected, the relative number of BrdU-positive cells after a six-hour incubation was approximately twice the relative number of cells undergoing mitosis during the same time period (FIG. 12A,C). In contrast to the similar proliferative activities in explants from NBCn1 KO and WT mice and in the presence of 5 μM S0859, VSMC proliferation was significantly lower (P<0.01, two-way ANOVA) when explants were investigated in absence than presence of CO₂/HCO₃⁻ (FIG. 12A).

It is generally proposed that an alkaline global pHᵢ stimulates cell proliferation and growth. When effects of NBCn1 KO, S0859, and CO₂/HCO₃⁻ omission on VSMC proliferation (FIG. 12A) and media hypertrophy (FIG. 9D) were related to the global pHᵢ measured in primary explants (FIG. 10C,F) and arteries (FIG. 8D), respectively, we found a significant positive correlation (FIG. 12D) supporting that low global pHᵢ inhibits VSMC proliferation.

Cell Density in Primary Explants

Cell size is relevant for tissue structure and related to VSMC phenotype. Furthermore, due to its role in ion translocation, NBCn1 may contribute to local and/or global volume regulation. We measured average projected cell areas based on cell densities in confluent areas of primary explants investigated in serum-supplemented medium. As shown in FIG. 12E, no significant difference was seen in projected cell areas between aortic explants from NBCn1 KO and WT mice.

VSMC Cell Death

Increased cell death would provide an alternative explanation for the decelerated wound healing in explants from NBCn1 KO mice and in explants treated with S0859. However, even after prolonged periods of investigation, cell integrity and morphology were typically well-maintained in aortic explants from NBCn1 KO and WT mice (FIG. 12F) and during treatment with 5 μM S0859 (FIG. 12G). The number of cells with normal morphology after 18 hours of time-lapse imaging relative to immediately after scratch infliction was also similar (P=0.40; unpaired, two-tailed Student's t-test) in carotid explants from NBCn1 KO (91±3%, n=11) and WT mice (96±4%, n=8). In contrast, treatment with 30 μM S0859 caused severe morphological changes over a period of 18 hours (FIG. 12G and Supplemental FIG. 9). Importantly, the morphological effects of 30 μM S0859 were independent of HCO₃⁻ transport, since similar effects were observed in the absence of CO₂/HCO₃⁻ (FIG. 12H). These findings show that 30 μM S0859 has effects on viability, which are not explained by actions on Na⁺,HCO₃⁻-cotransport, whereas inhibition of Na⁺,HCO₃⁻-cotransport does not affect cell survival under the conditions of the assay.

Local pH Regulation in Migrating VSMCs

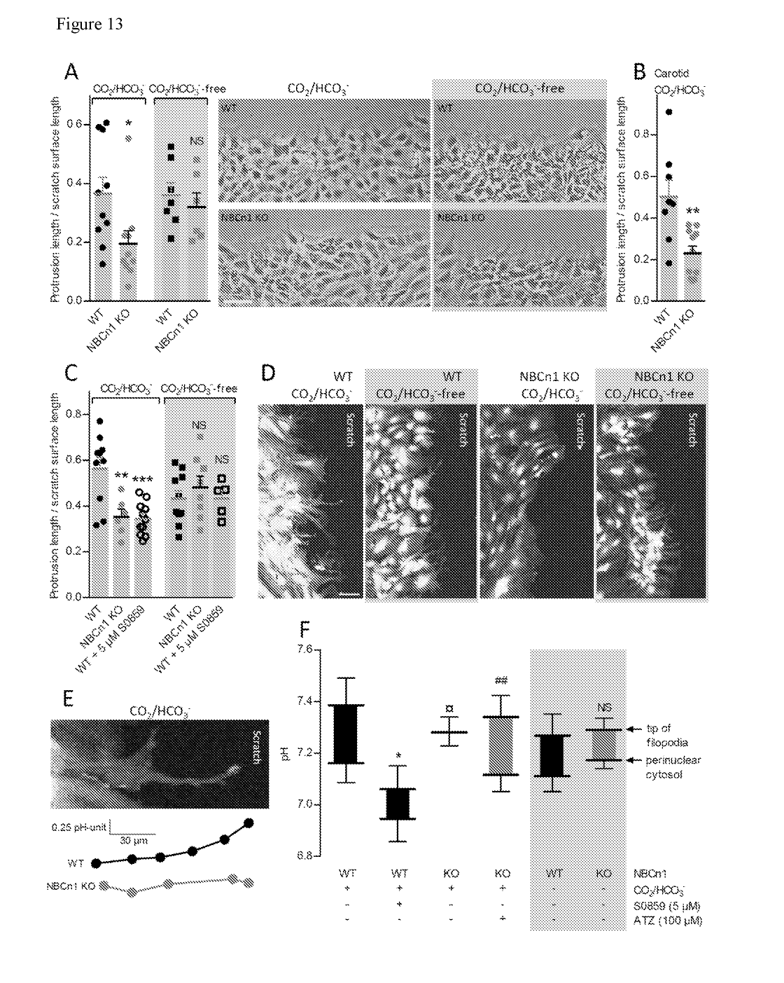

Migrating primary VSMCs were characterized by cell protrusions extending into the scratch-wound (FIG. 13A). The presence of filopodia was substantially reduced in explants from NBCn1 KO compared to WT mice when investigated in serum-supplemented medium (FIG. 13A,B) or PSS (FIG. 13C) containing CO₂/HCO₃⁻. When applied to CO₂/HCO₃⁻-containing PSS, 5 μM S0859 reduced the presence of filopodia in explants from WT mice (FIG. 13C). Under CO₂/HCO₃⁻-free conditions, the presence of filopodia was similar in VSMCs from NBCn1 KO and WT mice and unaffected by 5 μM S0859 (FIG. 13A,C).

Previous studies from non-vascular cell types propose that increased acid extrusion at the leading edge of migrating cells creates an axial pHᵢ gradient, which can facilitate actin polymerization, filopodia formation, and cell migration. To investigate pH gradients within cells, we measured pHᵢ -differences between the tip of filopodia and the general (perinuclear) cytosol two hours after scratch infliction. These experiments—performed in PSS—confirmed that whereas VSMCs from WT mice showed a similar abundance of filopodia in presence (FIG. 13D, first panel) and absence (FIG. 13D, second panel) of CO₂/HCO₃⁻,VSMCs from NBCn1 KO mice had substantially more or longer filopodia when incubated in absence than presence of CO₂/HCO₃⁻ (FIG. 13D, two right panels). With the intention of investigating local pHᵢ dynamics in the region of the filopodia, we incubated primary explants in absence of CO₂/HCO₃⁻ for two hours after wound infliction to allow for filopodia formation. We then investigated pHᵢ after an additional 15 minutes with or without CO₂/HCO₃⁻ or in the presence of pharmacological inhibitors (FIG. 13E,F). In explants from WT mice, pHᵢ at the tip of filopodia was ˜0.2 higher than in the perinuclear cytosol (FIG. 13F). In presence of CO₂/HCO₃⁻,this pHᵢ gradient was absent in explants from NBCn1 KO mice (FIG. 13E,F). Addition of 5 μM S0859 strongly acidified the migrating VSMCs and in particular shifted pHᵢ at the tip of filopodia to very low levels (FIG. 13F). Taken together, these findings support that under physiological conditions Na⁺,HCO₃⁻-cotransport via NBCn1 is required for establishing axial pHᵢ gradients that facilitate filopodia formation.

In absence of CO₂/HCO₃⁻, we detected axial pHᵢ gradients in explants from both NBCn1 KO and WT mice (FIG. 13F). This finding suggests that CO₂/HCO₃⁻-independent acid-base transport can establish pHᵢ gradients in VSMCs but only under artificial conditions without CO₂/HCO₃⁻. We hypothesized that lower concentrations of mobile intracellular buffers in absence of CO₂/HCO₃⁻ reduce the effective intracellular H⁺ mobility and thereby permit CO₂/HCO₃⁻-independent acid-base transporters to establish pHᵢ gradients. Consistent with this hypothesis, we found that 100 μM of the cell-permeable carbonic anhydrase inhibitor acetazolamide—which has been shown to reduce the apparent intracellular H⁺ mobility in cardiomyocytes—reestablishes axial pHᵢ gradients in explants from NBCn1 KO mice in presence of CO₂/HCO₃⁻ (FIG. 13F).

Role of Carbonic Anhydrases in Cell Migration and Filopodia

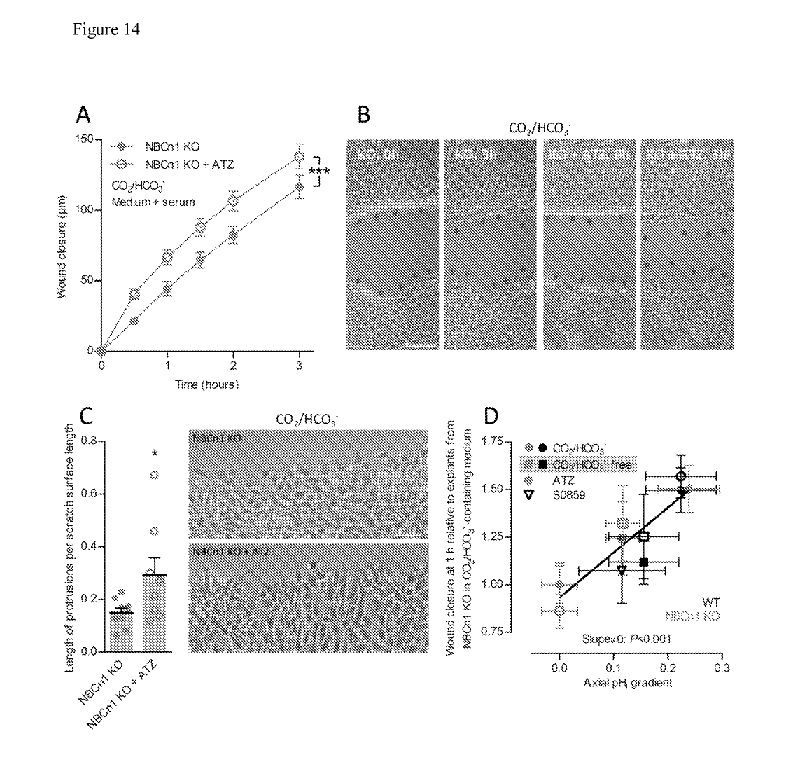

To test whether pHᵢ gradients in primary aortic explants facilitate migration, we next studied the effects of acetazolamide on wound healing in explants from NBCn1 KO mice. As shown in FIG. 14A, 100 μM acetazolamide increased the initial rate of wound healing. This effect is also illustrated in FIG. 14B and is of a magnitude approximately similar to the difference between aortic explants from NBCn1 KO and WT mice in presence of CO₂/HCO₃⁻ (FIG. 11A,B).

The effect of acetazolamide on wound healing in aortic explants from NBCn1 KO mice was paralleled by an increased abundance of filopodia (FIG. 14C). The ability of acetazolamide to reestablish axial pHᵢ gradients as well as filopodia and migration rates strongly supports their interdependency.

Correlation Between pHᵢ Gradients and Cell Migration

To further investigate the importance of the axial pHᵢ gradient, we plotted rates of wound healing as function of the size of the corresponding pHᵢ gradients. We reduced variation between different experimental series by expressing rates of wound healing relative to explants from NBCn1 KO mice in presence of CO₂/HCO₃⁻. As shown in FIG. 14D, there was a strong correlation between the axial pHᵢ gradient and the initial rate of wound healing.

Discussion

We show here that Na⁺,HCO₃⁻-cotransport via NBCn1 facilitates migration of VSMCs (FIG. 11) and remodeling of conduit arteries in response to reduced shear stress (FIG. 9). We demonstrate for the first time that migrating primary VSMCs display pHᵢ gradients along their axis of translocation, and we corroborate that these axial pHᵢ gradients promote filopodia formation and migration (FIGS. 13,14).

Regulation of pHᵢ is typically considered a homeostatic mechanism with the overall aim of maintaining constant pHᵢ despite changes in metabolism and extracellular acid-base status. The current study supports that pHᵢ changes can also serve signaling purposes and thus contribute to dynamic cell responses. By establishing pHᵢ gradients between the leading and rear end of migrating cells, NBCn1 likely coordinates the activity of pH sensitive proteins (e.g., talin and cofilin) that facilitate cell migration by modulating actin polymerization and focal adhesions. Although pHᵢ can fundamentally alter the function of almost all cellular proteins—and thus could appear too general for signaling purposes—the spatial restriction of pHᵢ demonstrated in the current study (FIG. 13E,F) allows for coordinated control of protein function specifically in a given region of the cell.

Whereas the role of Na⁺,HCO₃⁻-cotransporters for cell migration has been controversial, several studies—based primarily on non-vascular cells investigated without CO₂/HCO₃⁻—have implicated Na⁺/H⁺ exchanger NHE1 in cell migration. Because vascular cells rely heavily on CO₂/HCO₃⁻-dependent mechanisms, the use of CO₂/HCO₃⁻-containing solutions is critical for studies of pHᵢ regulation and its associated functional effects in the vascular system. In the current study, we investigated primary aortic and carotid artery explants. During cell culture, VSMCs are known to undergo phenotypic shift from a contractile (typical for healthy arteries) to a synthetic or proliferative phenotype that can also be observed during vascular remodeling and in atherosclerotic plaques. Our results suggest that CO₂/HCO₃⁻ modifies local VSMC pHᵢ by (a) changing relative acid-base transport activities at different subcellular locations and (b) increasing buffer mobility—and hence the effective intracellular H⁺ mobility—which can dissipate pHᵢ gradients (FIG. 13F). We propose that spatial differences in local expression and/or activity of acid-base transporters and carbonic anhydrases—relative to the associated cytosolic volume—contribute to the dynamic formation and dissipation of pH gradients. It is possible that metabolic acid production also varies spatially: in endothelial cells, for instance, filopodia are rich in glycolytic enzymes but devoid of mitochondria. This compartmentalization of metabolism and the associated production of acidic waste products may contribute to the formation of axial pHᵢ gradients.

It has been discussed whether Na⁺/H⁺-exchangers affect cell migration due to transport of acid-base equivalents or as a result of transport-independent functions (e.g., changes in intracellular signaling cascades or binding to cytoskeletal or extracellular matrix components). Since NBCn1 and NHE1 perform equivalent transport functions but are unlikely to mediate the same transport-independent effects, our finding that NBCn1 contributes to cell migration supports a role for transport of acid-base equivalents. The importance of acid-base transport is further supported by the inability of NBCn1 KO and 5 μM S0859 to interfere with cell migration in absence of CO₂/HCO₃⁻ (FIG. 11F,G) when the transport function of NBCn1 is already inhibited. Still, because interventions that interfere with acid-base transport activity may alter the ability of the transporters to link (e.g., via ezrin-radixin-moesin proteins) to the cytoskeleton, it cannot be excluded that disrupted binding to interacting proteins may contribute to the impaired cell migration after inhibition of NBCn1.

We show here that NBCn1 KO reduces media hypertrophy following carotid artery ligation in vivo (FIG. 9D). In apparent contrast, NBCn1 KO did not affect cell proliferation or cell size in vitro (FIG. 12A-E). This dissimilarity between in vivo and in vitro conditions may reflect limitations of 2-dimensional culture conditions compared to the complex 3-dimensional structure of the arterial wall, differences in the release of chemokines and growth factors caused by scratch wounds vs. altered flow conditions or the absence vs. presence of interactions with other cell types (e.g., immune cells). However, the larger NBCn1-dependent difference in global steady-state pHᵢ (FIG. 8D vs. 10C) and Na⁺,HCO₃⁻-cotransport activity (FIG. 8C vs. 10A) for carotid arteries compared to primary explants suggests that functional upregulation of other Na⁺,HCO₃⁻-cotransporters during cell culture can account for the difference in NBCn1-dependent hypertrophy and proliferation. In congruence, omission of CO₂/HCO₃⁻ and consequent inhibition all HCO₃⁻-dependent transport mechanisms in primary explants produced a greater pHᵢ decrease than KO of NBCn1 (FIG. 10C) and moreover inhibited VSMC proliferation in vitro (FIG. 12A). Together, our observations in the arterial wall as well as in primary explants show significant correlation between global VSMC pH cell proliferation, and media hypertrophy (FIG. 12D). This finding is in agreement with the recent finding that KO of NBCn1 inhibits cancer cell proliferation in primary breast carcinomas. Although it is difficult to extrapolate from 2-dimensional culture conditions to in vivo circumstances—and quantitative comparisons are at best speculative—the phenomenological congruence between our in vivo and in vitro observations supports a causative link between the decelerating effect of NBCn1 KO on wound healing in vitro and the reduced remodeling in vivo.

Pharmacological agents that selectively inhibit Na⁺,HCO₃⁻-cotransporters have been lacking, and although S0859 provides advantages compared to previously employed compounds (such as 4,4-diisothiocyanatostilbene-2,2′-disulphonate, DIDS), the current study shows that S0859 should be used with caution. We see convincing CO₂/HCO₃⁻-dependency of S0859 only in the low-μM range. At higher concentrations, deleterious effects of S0859 on cell morphology and survival (FIG. 12G,H) preclude meaningful conclusions regarding integrated cellular consequences of inhibiting Na⁺,HCO₃⁻-cotransport. The concentration required for complete inhibition of Na⁺,HCO₃⁻-cotransport is close to the concentration causing cell damage (compare FIG. 10D,E and FIG. 12G,H). These findings and previous evidence that S0859 binds to plasma and presumably extracellular connective tissue components limit the applicability of the compound to carefully controlled in vitro investigations of isolated cells.

In conclusion, we find that Na⁺,HCO₃⁻-cotransport mediated via NBCn1 plays a central role for VSMC acid extrusion and steady-state pHᵢ control in mouse conduit arteries. In particular, we demonstrate that NBCn1 creates pHᵢ gradients along the axis of migration, facilitates migration of VSMCs, and promotes remodeling of carotid arteries in response to altered shear stress. These findings provide new mechanistic insights on how transmembrane acid-base transport can modify remodeling of conduit arteries and identify NBCn1 as a novel target for occlusive artery disease, such as atherosclerosis and/or restenosis.

### Example 3

Methods

Development of Polyclonal Antibodies

Based on predicted antigenic determinants in extracellular loop 3 of human NBCn1 (FIG. 17), we selected two overlapping peptides covering the regions of high estimated antigenicity: NBCn1_EL3h1.1: [Hz]-HNNLDKLTSYSCVCTEPPNPSNETLAQWKKDNITA-amide (SEQ ID NO: 14) and NBCn1_EL3h2.1: [Hz]-LAQWKKDNITAHNISWRNLTVSECKKLRGVFLGSA-amide (SEQ ID NO: 15).

The hydrazine (Hz)-labelled peptides were synthesized and keyhole limpet hemocyanin (KLH)-conjugated by 21^{st} Century Biochemicals (MA, USA). The KLH-conjugated peptides in Complete Freund's Adjuvant were each injected into two specific pathogen-free New Zealand rabbits. The rabbits were boosted through subsequent injections of the same KLH-conjugated peptide in Incomplete Freund's Adjuvant after 2, 4, 6 and 8 weeks. Serum was collected from blood sampled before first immunization (pre-immune serum) and at week 7, 8, 10 and 11.

Binding Assays and Affinity Purification

The collected serum samples were tested for binding to the synthesized peptides conjugated to bovine serum albumin (BSA) based on enzyme-linked immunosorbent assay (ELISA) analysis. An anti-BSA antibody (A11133, Life Technologies) was used as positive control and pre-immune serum served as negative control. Flat-bottomed 96-well cell culture plates (734-2327, VWR) were coated overnight at 4° C. with BSA-conjugated peptide (diluted to 2 μg/mL in phosphate buffered saline (PBS); 60 μL per well) and then blocked with 15% fetal bovine serum (FBS; #S0115, Biochrom AG; 75 μL per well) in PBS for 1 hour at 37° C. Between each step, the plates were washed 4 times with 0.05% Tween-20 in PBS. Sera and anti-BSA antibody were diluted in PBS, added to wells in duplicates (50 μL per well), and incubated for 1 hour at 37° C. After washing, the plates were incubated with horseradish peroxidase-conjugated secondary goat anti-rabbit IgG (#70745, Cell Signalling; 50 μL per well) for 1 hour at 37° C. After further washing, the plates were developed by incubation with the horseradish peroxidase substrate 3,3′,5,5′-tetramethylbenzidine (002023, Life Technologies; 50 μL per well) for 30 minutes at 37° C. Reactions were stopped by addition of 100 μL 1 M H₂SO₄ to each well. Absorbances were measured at 450 nm on a PowerWave 340 microplate spectrophotometer (BioTek) using 620 nm readings as reference.

Sera with the highest titers were selected for affinity purification: we used pools of all 4 bleeds collected from rabbits injected with NBCn1_EL3h1.1 (B1-B4 in FIG. 18A) and pools of the last two bleeds collected from rabbits injected with NBCn1_EL3h2.1 (B3 and B4 in FIG. 18B). Affinity purification was performed by 21^{st} Century Biochemicals using the synthetic peptides immobilized on optimized affinity resin. Lyophilized antibodies were stored at −20° C. until use.

Functional Testing of Affinity-Purified Antibodies

The affinity-purified antibodies were tested functionally as inhibitors of Na⁺,HCO₃⁻-cotransport in MCF-7 human breast cancer cells. Because net acid extrusion from MCF-7 cells occurs through a combination of NBCn1-mediated Na⁺,HCO₃⁻-cotransport and NHE1-mediated Na⁺/H⁺-exchange (Lauritzen et al., 2010), we evaluated the degree of Na⁺,HCO₃⁻-cotransport inhibition by comparing the effect of the antibodies to that of the non-selective Na⁺,HCO₃⁻-cotransport inhibitor S0859 (Steinkamp et al., 2015, Larsen et al., 2012). Regulation of intracellular pH (pHᵢ) was also investigated in the absence of CO₂/HCO₃⁻.

MCF-7 cells (Lauritzen et al., 2010) were grown in RPMI-1640 medium (#61870-010, Gibco) supplemented with 6% FBS (#S0115, Biochrom AG), 1% penicillin/streptomycin (#15140-122, Gibco), 1 μg/mL puromycin (#P9620, Sigma), and 200 μg/mL G418 (#G8168, Sigma). Cells were maintained at 37° C. in an atmosphere of 5% CO₂ and used at passages 15-21. MCF-7 cells were seeded on cover glasses coated for 2 hours with 0.2% autoclaved gelatin (#G2625, Sigma) and investigated after 2-3 days. MCF-7 cells were loaded with 3 μM BCECF-AM (Invitrogen) for 20-30 minutes at 37° C. The preparations were mounted in a custom-built chamber on the stage of an Olympus IX70 or Nikon Diaphot 200 microscope and excited alternately at approximately 490 nm and 440 nm. Emission light was collected at 510 nm with CCD-based fluorescence imaging systems controlled through EasyRatioPro (Photon Technology International, NJ, USA) or VisiView (Visitron Systems, Germany) software.

Intracellular acidification was induced by the NH₄⁺-prepulse technique (Boron and De Weer, 1976): 20 mM NH₄Cl was added to the experimental bath solution for 15 minutes before it was washed out to a Na⁺-free solution in order to first evaluate the rate of Na⁺-independent net acid extrusion. Then, Na⁺ was returned to the bath solution to activate Na⁺-dependent net acid extrusion mechanisms. The BCECF fluorescence ratios were taken as a measure of pHᵢ. Na⁺-dependent pHᵢ recovery rates calculated over 30-second intervals were evaluated based on the initial increase in pHᵢ recovery rate after addition of extracellular Na⁺ and expressed relative to control conditions without antibody or S0859.

The affinity-purified anti-NBCn1 antibodies were solubilized in distilled water and added to the experimental solutions (at 1:100 dilution) from the time of NH₄Cl addition until the end of the experiment. S0859 was solubilized in DMSO and added to the bath solution at a concentration of 30 μM from the time of NH₄Cl washout until the end of the experiment. The final [DMSO] in the bath was kept below 0.1%.

The physiological saline solution used for pHᵢ recordings contained (in mM): 140 Na⁺, 4 K⁺, 1.6 Ca²⁺, 1.2 Mg²⁺, 122 Cl⁻, 24 HCO₃⁻, 1.2 SO₄²⁻. 1.18 H₂PO₄⁻, 10 HEPES, 5.5 glucose, 0.03 EDTA. In CO₂/HCO₃⁻-free solutions, HCO₃⁻ was replaced with equimolar amounts of Cl⁻. In Na⁺-free solutions, Na⁺ was replaced with equimolar amounts of N-methyl-D-glucammonium; except for NaHCO₃, which was replaced with choline-HCO₃. CO₂/HCO₃⁻-containing solutions were aerated with 5% CO₂/balance air, whereas CO₂/HCO₃⁻-free solutions were bubbled with nominally CO₂-free air. All solutions were adjusted to pH 7.4 at 37° C. Solutions contained 5 mM probenecid to inhibit BCECF extrusion by the organic anion transporter.

Statistics

Data are expressed as mean±SEM, n equals number of experiments. Data were compared by one-way ANOVA followed by Dunnett's post-test or by unpaired, two-tailed Student's t-test. P<0.05 was considered statistically significant. Statistical analyses were performed using GraphPad Prism 5.02 software.

Results

Sera from rabbits immunized with the KLH-conjugated peptides (corresponding to fragments of the extracellular loop 3 of human NBCn1, see FIG. 17) showed strong titers against the equivalent BSA-conjugated peptides (FIG. 18).

Biological Testing for Na⁺,HCO₃⁻-Cotransport Inhibitory Function

Extracellular application of 20 mM NH₄Cl to MCF-7 human breast cancer cells initially caused abrupt intracellular alkalinization (due to fast influx of NH₃) followed by gradual pHᵢ recovery towards resting levels (due to NH₄⁺ uptake and activation of base extrusion mechanisms; FIG. 19A,B). Upon washout of NH₄Cl from the experimental bath solution, NH₃ rapidly leaves the cells shifting the NH₄⁺⇄NH₃+H⁺ equilibrium to the right, liberating H⁺ and causing the desired intracellular acidification. We tested the ability of the MCF-7 cells to activate net acid extrusion mechanisms and recover pHᵢ from this intracellular acidification in the presence and absence of the affinity-purified anti-NBCn1_EL3h1.1 and anti-NBCn1_EL3h2.1 polyclonal antibodies.

As shown in FIG. 19A, we first evaluated the rate of Na⁺-independent pHᵢ recovery (under Na⁺-free extracellular conditions) and then the increase in pHᵢ recovery rate after Na⁺ was returned to the experimental bath solution. The anti-NBCn1_EL3h1.1 and anti-NBCn1_EL3h2.1 antibodies both inhibited Na⁺-dependent net acid extrusion from MCF-7 human breast cancer cells in the presence of CO₂/HCO₃⁻ (FIG. 19A,C). Most notably, the inhibition achieved with the affinity-purified anti-NBCn1_EL3h2.1 antibody (FIG. 19A,C) was of similar magnitude to that observed with 30 μM S0859 (FIG. 19B,D; see also (Larsen et al., 2012, Lauritzen et al., 2010, Steinkamp et al., 2015)) or following siRNA-mediated knockdown of NBCn1 (Lauritzen et al., 2010). Omission of CO₂/HCO₃⁻ under control conditions reduced the Na⁺-dependent pHᵢ recovery rate to the level seen with the anti-NBCn1_EL3h2.1 antibody in the presence of CO₂/HCO₃⁻ (FIG. 19C). This was also the case at low pHᵢ where CO₂/HCO₃⁻ contributes only marginally to intracellular buffering. Taken together, these findings are consistent with the anti-NBCn1_EL3h2.1 antibody completely inhibiting NBCn1-mediated Na⁺,HCO₃⁻-cotransport in MCF-7 cells.

The remaining net acid extrusion after inhibition of Na⁺,HCO₃⁻-cotransport in MCF-7 cells has previously been shown to be due to Na⁺/H⁺-exchange, as it is inhibited by the Na⁺/H⁺-exchange inhibitor 5-(N-Ethyl-N-isopropyl)amiloride or by siRNA-mediated knockdown of NHE1 (Lauritzen et al., 2010). In agreement, no effect of the anti-NBCn1_EL3h2.1 antibody was seen on the Na⁺-dependent pHᵢ recovery rate in the absence of CO₂/HCO₃⁻ (FIG. 19C).

Discussion

We developed polyclonal anti-NBCn1 antibodies against the predicted extracellular loop 3 of human NBCn1. Comparing the degree to which our newly developed anti-NBCn1 antibodies inhibit net acid extrusion to previously reported inhibitory effects of S0859 and siRNA-mediated knockdown of NBCn1 (Lauritzen et al., 2010, Larsen et al., 2012, Steinkamp et al., 2015), we conclude that the developed anti-NBCn1_EL3h2.1 antibody is able to completely inhibit NBCn1-mediated Na⁺,HCO₃⁻-cotransport during intracellular acidification of MCF-7 human breast cancer cells. This conclusion is further supported by the observation that omission of CO₂/HCO₃⁻ inhibited net acid extrusion to the same relative extent as addition of the anti-NBCn1_EL3h2.1 antibody in the presence of CO₂/HCO₃⁻. Furthermore, the anti-NBCn1_EL3h2.1 antibody did not affect Na⁺/H⁺-exchange activity evaluated as the Na⁺-dependent pHᵢ recovery rate in the absence of CO₂/HCO₃⁻.

In conclusion, our data demonstrate for the first time that it is possible to generate antibodies against extracellular loop 3 of human NBCn1 and that these antibodies have the capacity to fully inhibit NBCn1-mediated Na⁺,HCO₃⁻-cotransport. Considering the poor selectivity of S0859 (Heidtmann et al., 2015) (FIG. 12) and the inability of S0859 to inhibit Na⁺,HCO₃⁻-cotransport in tissue preparations (Steinkamp et al., 2015, Larsen et al., 2012), the development of inhibitory antibodies against NBCn1 opens up new therapeutic possibilities.

### REFERENCE LIST

Boron, W. F. & De Weer, P. 1976. Intracellular pH transients in squid giant axons caused by CO₂, NH₃, and metabolic inhibitors. J Gen Physiol, 67, 91-112.
Heidtmann, H., Ruminot, I., Becker, H. M. & Deitmer, J. W. 2015. Inhibition of monocarboxylate transporter by N-cyanosulphonamide S0859. Eur J Pharmacol, 762, 344-349.
Larsen, A. M., Krogsgaard-Larsen, N., Lauritzen, G., Olesen, C. W., Honoré Hansen, S., Boedtkjer, E., Pedersen, S. F. & Bunch, L. 2012. Gram-scale solution-phase synthesis of selective sodium bicarbonate co-transport inhibitor S0859: in vitro efficacy studies in breast cancer cells. Chem Med Chem, 7, 1808-1814.
Lauritzen, G., Jensen, M. B., Boedtkjer, E., Dybboe, R., Aalkjaer, C., Nylandsted, J. & Pedersen, S. F. 2010. NBCn1 and NHE1 expression and activity in ΔNErbB2 receptor-expressing MCF-7 breast cancer cells: Contributions to pH₁ regulation and chemotherapy resistance. Exp Cell Res, 316, 2538-2553.
Steinkamp, A.-D., Seling, N., Lee, S., Boedtkjer, E. & Bolm, C. 2015. Synthesis of N-cyano-substituted sulfilimine and sulfoximine derivatives of S0859 and their biological evaluation as sodium bicarbonate co-transport inhibitors. Med Chem Comm, 6, 2163-2169.

Sequences

SEQ ID NO: 1
Human NBCn1 DNA sequence variant a cloned from
breast cancer tissue. The underlined 5′-region
indicates a region, which differs from SEG ID
NO: 2.
atggaaagatttcgtctggagaagaagttacctggtcctgatgaagaag
ctgttgtggatcttggcaaaactagctcaactgtgaacaccaagtttga
aaaagaagaactagaaagtcatagagctgtatatattggtgttcacgtc
ccgtttagtaaagagagtcgtcggcgtcataggcatcgcggacacaaac
atcaccaccggagaagaaaagataaagaatcagataaagaagatggacg
ggaatctccttcttatgatacaccatcccagagagttcagtttatcctt
ggtactgaagatgatgatgaagaacatattccccatgatctcttcacgg
aaatggatgaactgtgttacagagatggagaagaatatgaatggaaaga
aactgctagatggctgaaatttgaagaggatgttgaagatggcggtgac
cgatggagtaaaccttatgtggcaactctctctttgcacagtctttttg
aactaaggagttgcatcctcaatggaacagtcatgctggatatgagagc
aagcactctagatgaaatagcagatatggtattagacaacatgatagct
tctggccaattagacgagtccatacgagagaatgtcagagaagctcttc
tgaagagacatcatcatcagaatgagaaaagattcaccagtcggattcc
tcttgttcgatcttttgcagatataggcaagaaacattctgaccctcac
ttgcttgaaaggaatggtattttggcctctccccagtctgctcctggaa
acttggacaatagtaaaagtggagaaattaaaggtaatggaagtggtgg
aagcagagaaaatagtactgttgacttcagcaaggttgatatgaatttc
atgagaaaaattcctacgggtgctgaggcatccaacgtcctggtgggcg
aagtagactttttggaaaggccaataattgcatttgtgagactggctcc
tgctgtcctccttacagggttgactgaggtccctgttccaaccaggttt
ttgtttttgttattgggtccagcgggcaaggcaccacagtaccatgaaa
ttggacgatcaatagccactctcatgacagatgagattttccatgatgt
agcttataaagcaaaagacagaaatgacctcttatctggaattgatgaa
tttttagatcaagtaactgtcctacctccaggagagtgggatccttcta
tacgcatagaaccaccaaaaagtgtcccttctcaggaaaagagaaagat
tcctgtgtttcacaatggatctacccccacactgggtgagactcctaaa
gaggccgctcatcatgctgggcctgagctacagaggactggacggcttt
ttggtggtttgatacttgacatcaaaaggaaagcaccttttttcttgag
tgacttcaaggatgcattaagcctgcagtgcctggcctcgattcttttc
ctatactgtgcctgtatgtctcctgtaatcacttttggagggctgcttg
gagaagctacagaaggcagaataagtgcaatagagtctctttttggagc
atcattaactgggattgcctattcattgtttgctgggcaacctctaaca
atattggggagcacaggtccagttctagtgtttgaaaaaattttatata
aattctgcagagattatcaactttcttatctgtctttaagaaccagtat
tggtctgtggacttcttttttgtgcattgttttggttgcaacagatgca
agcagccttgtgtgttatattactcgatttacagaagaggcttttgcag
cccttatttgcatcatattcatctacgaggctttggagaagctctttga
tttaggagaaacatatgcatttaatatgcacaacaacttagataaactg
accagctactcatgtgtatgtactgaacctccaaaccccagcaatgaaa
ctctagcacaatggaagaaagataatataacagcacacaatatttcctg
gagaaatcttactgtttctgaatgtaaaaaacttcgtggtgtattcttg
gggtcagcttgtggtcatcatggaccttatattccagatgtgctctttt
ggtgtgtcatcttgtttttcacaacattttttctgtcttcattcctcaa
gcaatttaagaccaagcgttactttcctaccaaggtgcgatcgacaatc
agtgattttgctgtatttctcacaatagtaataatggttacaattgact
accttgtaggagttccatctcctaaacttcatgttcctgaaaaatttga
gcctactcatccagagagagggtggatcataagcccactgggagataat
ccttggtggaccttattaatagctgctattcctgctttgctttgtacca
ttctcatctttatggatcaacaaatcacagctgtaattataaacagaaa
ggaacacaaattgaagaaaggagctggctatcaccttgatttgctcatg
gttggcgttatgttgggagtttgctctgtcatgggacttccatggtttg
tggctgcaacagtgttgtcaataagtcatgtcaacagcttaaaagttga
atctgaatgttctgctccaggggaacaacccaagtttttgggaattcgt
gaacagcgggttacagggctaatgatttttattctaatgggcctctctg
tgttcatgacttcagtcctaaagtttattccaatgcctgttctgtatgg
tgttttcctttatatgggagtttcctcattaaaaggaatccagttattt
gaccgtataaaattatttggaatgcctgctaagcatcagcctgatttga
tatacctccgttatgtgccgctctggaaggtccatattttcacagtcat
tcagcttacttgtttggtccttttatgggtgataaaagtttcagctgct
gcagtggtttttcccatgatggttcttgcattagtgtttgtgcgcaaac
tcatggacctgtgtttcacgaagagagaacttagttggcttgatgatct
tatgccagaaagtaagaaaaagaaagaagatgacaaaaagaaaaaagag
aaagaggaagctgaacggatgcttcaagatgatgatgatactgtgcacc
ttccatttgaagggggaagtctcttgcaaattccagtcaaggccctaaa
atatagtcctgataaacctgtgagtgtgaaaataagttttgaagatgaa
ccaagaaagaaatacgtggatgctgaaacttcattatagaattgaacca
agaggcattatacatatagatatatacatatgtaatgtgtgcgtatcat
gtcactatatataagaatattgtatgtcatgctgtttatgtgtgactac
cgggtttttaaaagtagt
SEQ ID NO: 2
Human NBCn1 DNA sequence variant b cloned from
breast cancer tissue. The underlined 5′-region
indicates a region, which differs from SEG ID
NO: 1.
atggaggctgatggggccggcgagcagatgagaccgctactcacccggg
gtcctgatgaagaagctgttgtggatcttggcaaaactagctcaactgt
gaacaccaagtttgaaaaagaagaactagaaagtcatagagctgtatat
attggtgttcacgtcccgtttagtaaagagagtcgtcggcgtcataggc
atcgcggacacaaacatcaccaccggagaagaaaagataaagaatcaga
taaagaagatggacgggaatctccttcttatgatacaccatcccagaga
gttcagtttatccttggtactgaagatgatgatgaagaacatattcccc
atgatctcttcacggaaatggatgaactgtgttacagagatggagaaga
atatgaatggaaagaaactgctagatggctgaaatttgaagaggatgtt
gaagatggcggtgaccgatggagtaaaccttatgtggcaactctctctt
tgcacagtctttttgaactaaggagttgcatcctcaatggaacagtcat
gctggatatgagagcaagcactctagatgaaatagcagatatggtatta
gacaacatgatagcttctggccaattagacgagtccatacgagagaatg
tcagagaagctcttctgaagagacatcatcatcagaatgagaaaagatt
caccagtcggattcctcttgttcgatcttttgcagatataggcaagaaa
cattctgaccctcacttgcttgaaaggaatggtattttggcctctcccc
agtctgctcctggaaacttggacaatagtaaaagtggagaaattaaagg
taatggaagtggtggaagcagagaaaatagtactgttgacttcagcaag
gttgatatgaatttcatgagaaaaattcctacgggtgctgaggcatcca
acgtcctggtgggcgaagtagactttttggaaaggccaataattgcatt
tgtgagactggctcctgctgtcctccttacagggttgactgaggtccct
gttccaaccaggthttgtttttgttattgggtccagcgggcaaggcacc
acagtaccatgaaattggacgatcaatagccactctcatgacagatgag
attttccatgatgtagcttataaagcaaaagacagaaatgacctcttat
ctggaattgatgaatttttagatcaagtaactgtcctacctccaggaga
gtgggatccttctatacgcatagaaccaccaaaaagtgtcccttctcag
gaaaagagaaagattcctgtgtttcacaatggatctacccccacactgg
gtgagactcctaaagaggccgctcatcatgctgggcctgagctacagag
gactggacggctttttggtggtttgatacttgacatcaaaaggaaagca
ccttttttcttgagtgacttcaaggatgcattaagcctgcagtgcctgg
cctcgattcttttcctatactgtgcctgtatgtctcctgtaatcacttt
tggagggctgcttggagaagctacagaaggcagaataagtgcaatagag
tctctttttggagcatcattaactgggattgcctattcattgtttgctg
ggcaacctctaacaatattggggagcacaggtccagttctagtgtttga
aaaaattttatataaattctgcagagattatcaactttcttatctgtct
ttaagaaccagtattggtctgtggacttcttttttgtgcattgttttgg
ttgcaacagatgcaagcagccttgtgtgttatattactcgatttacaga
agaggcttttgcagcccttatttgcatcatattcatctacgaggctttg
gagaagctctttgatttaggagaaacatatgcatttaatatgcacaaca
acttagataaactgaccagctactcatgtgtatgtactgaacctccaaa
ccccagcaatgaaactctagcacaatggaagaaagataatataacagca
cacaatatttcctggagaaatcttactgtttctgaatgtaaaaaacttc
gtggtgtattcttggggtcagcttgtggtcatcatggaccttatattcc
agatgtgctcttttggtgtgtcatcttgtttttcacaacattttttctg
tcttcattcctcaagcaatttaagaccaagcgttactttcctaccaagg
tgcgatcgacaatcagtgattttgctgtatttctcacaatagtaataat
ggttacaattgactaccttgtaggagttccatctcctaaacttcatgtt
cctgaaaaatttgagcctactcatccagagagagggtggatcataagcc
cactgggagataatccttggtggaccttattaatagctgctattcctgc
tttgctttgtaccattctcatctttatggatcaacaaatcacagctgta
attataaacagaaaggaacacaaattgaagaaaggagctggctatcacc
ttgatttgctcatggttggcgttatgttgggagtttgctctgtcatggg
acttccatggtttgtggctgcaacagtgttgtcaataagtcatgtcaac
agcttaaaagttgaatctgaatgttctgctccaggggaacaacccaagt
ttttgggaattcgtgaacagcgggttacagggctaatgatttttattct
aatgggcctctctgtgttcatgacttcagtcctaaagtttattccaatg
cctgttctgtatggtgttttcctttatatgggagtttcctcattaaaag
gaatccagttatttgaccgtataaaattatttggaatgcctgctaagca
tcagcctgatttgatatacctccgttatgtgccgctctggaaggtccat
attttcacagtcattcagcttacttgtttggtccttttatgggtgataa
aagtttcagctgctgcagtggtttttcccatgatggttcttgcattagt
gtttgtgcgcaaactcatggacctgtgthcacgaagagagaacttagtt
ggcttgatgatcttatgccagaaagtaagaaaaagaaagaagatgacaa
aaagaaaaaagagaaagaggaagctgaacggatgcttcaagatgatgat
gatactgtgcaccttccatttgaagggggaagtctcttgcaaattccag
tcaaggccctaaaatatagtgttgatccctcaattgttaacatatcaga
tgaaatggccaaaactgcacagtggaaggcactttccatgaatactgag
aatgccaaagtaaccagatctaacatgagtcctgataaacctgtgagtg
tgaaaataagttttgaagatgaaccaagaaagaaatacgtggatgctga
aacttcattatagaattgaaccaagaggcattatacatatagatatata
catatgtaatgtgtgcgtatcatgtcactatatataagaatattgtatg
tcatgctgtttatgtgtgactaccgggtttttaaaagtagtgtctggag
tttgtaatgagcaccgtggagactatgtatttaatgaaatgctctcttt
gaagtgaggtacatggttctt
SEQ ID NO: 3
Human NBCn1 Peptide sequence (variant a) cloned
from breast cancer tissue. The underlined N-
terminal region indicates where SEG ID NO: 3
differ from SEG ID NO: 4.
MERFRLEKKLPGPDEEAVVDLGKTSSTVNTKFEKEELESHRAVYIGVHV
PFSKESRRRHRHRGHKHHHRRRKDKESDKEDGRESPSYDTPSQRVQFIL
GTEDDDEEHIPHDLFTEMDELCYRDGEEYEWKETARWLKFEEDVEDGGD
RWSKPYVATLSLHSLFELRSCILNGTVMLDMRASTLDEIADMVLDNMIA
SGQLDESIRENVREALLKRHHHQNEKRFTSRIPLVRSFADIGKKHSDPH
LLERNGILASPQSAPGNLDNSKSGElKGNGSGGSRENSTVDFSKVDMNF
MRKIPTGAEASNVLVGEVDFLERPIIAFVRLAPAVLLTGLTEVPVPTRF
LFLLLGPAGKAPQYHEIGRSIATLMTDEIFHDVAYKAKDRNDLLSGIDE
FLDQVTVLPPGEWDPSIRIEPPKSVPSQEKRKIPVFHNGSTPTLGETPK
EAAHHAGPELQRTGRLFGGLILDIKRKAPFFLSDFKDALSLQCLASILF
LYCACMSPVITFGGLLGEATEGRISAIESLFGASLTGIAYSLFAGQPLT
ILGSTGPVLVFEKILYKFCRDYQLSYLSLRTSIGLVVTSFLCIVLVATD
ASSLVCYITRFTEEAFAALICIIFIYEALEKLFDLGETYAFNMHNNLDK
LTSYSCVCTEPPNPSNETLAQWKKDNITAHNISWRNLTVSECKKLRGVF
LGSACGHHGPYIPDVLFWCVILFFTTFFLSSFLKQFKTKRYFPTKVRST
ISDFAVFLTIVIMVTIDYLVGVPSPKLHVPEKFEPTHPERGWIISPLGD
NPWWTLLIAAIPALLCTILIFMDQQITAVIINRKEHKLKKGAGYHLDLL
MVGVMLGVCSVMGLPWFVAATVLSISHVNSLKVESECSAPGEQPKFLGI
REQRVTGLMIFlLMGLSVFMTSVLKFIPMPVLYGVFLYMGVSSLKGIQL
FDRIKLFGMPAKHQPDLIYLRYVPLWKVHIFTVIQLTCLVLLWVIKVSA
AAVVFPMMVLALVFVRKLMDLCFTKRELSWLDDLMPESKKKKEDDKKKK
EKEEAERMLQDDDDTVHLPFEGGSLLQIPVKALKYSPDKPVSVKISFED
EPRKKYVDAETSL*
SEQ ID NO: 4
Human NBCn1 Peptide sequence (variant b) cloned
from breast cancer tissue. The underlined N-
terminal region indicates where SEG ID NO: 3
differ from SEG ID NO: 4.
MEADGAGEQMRPLLTRGPDEEAVVDLGKTSSTVNTKFEKEELESHRAVY
IGVHVPFSKESRRRHRHRGHKHHHRRRKDKESDKEDGRESPSYDTPSQR
VQFILGTEDDDEEHIPHDLFTEMDELCYRDGEEYEWKETARWLKFEEDV
EDGGDRWSKPYVATLSLHSLFELRSCILNGTVMLDMRASTLDEIADMVL
DNMIASGQLDESIRENVREALLKRHHHQNEKRFTSRIPLVRSFADIGKK
HSDPHLLERNGILASPQSAPGNLDNSKSGEIKGNGSGGSRENSTVDFSK
VDMNFMRKIPTGAEASNVLVGEVDFLERPIAFVRLAPAVLLTGLTEVPV
PTRFLFLLLGPAGKAPQYHEIGRSIATLMTDEIFHDVAYKAKDRNDLLS
GIDEFLDQVTVLPPGEWDPSIRIEPPKSVPSQEKRKIPVFHNGSTPTLG
ETPKEAAHHAGPELQRTGRLFGGLILDIKRKAPFFLSDFKDALSLQCLA
SILFLYCACMSPVITFGGLLGEATEGRISAIESLFGASLTGIAYSLFAG
QPLTILGSTGPVLVFEKILYKFCRDYQLSYLSLRTSIGLVVTSFLCIVL
VATDASSLVCYITRFTEEAFAALICIIFIYEALEKLFDLGETYAFNMHN
NLDKLTSYSCVCTEPPNPSNETLAQWKKDNITAHNISWRNLTVSECKKL
RGVFLGSACGHHGPYIPDVLFWCVILFFTTFFLSSFLKQFKTKRYFPTK
VRSTISDFAVFLTIVIMVTIDYLVGVPSPKLHVPEKFEPTHPERGWIIS
PLGDNPWWTLLIAAIPALLCTILIFDQQITAVIINRKEHKLKKGAGYHL
DLLMVGVMLGVCSVMGLPWFVAATVLSISHVNSLKVESECSAPGEQPKF
LGIREQRVTGLMIFILMGLSVFMTSVLKFIPMPVLYGVFLYMGVSSLKG
IQLFDRIKLFGMPAKHQPDLIYLRYVPLWKVHIFTVIQLTCLVLLWVIK
VSAAAVVFPMMVLALVFVRKLMDLCFTKRELSWLDDLMPESKKKKEDDK
KKKEKEEAERMLQDDDDTVHLPFEGGSLLQIPVKALKYSVDPSIVNISD
EMAKTAQWKALSMNTENAKVTRSNMSPDKPVSVKISFEDEPRKKYVDAE
TSL*
SEQ ID NO: 5
EL2, human and mouse:
SPVITFGGLLGEATEGRISAIESLFGASLT
SEQ ID NO: 6
EL3, human:
KLFDLGETYAFNMHNNLDKLTSYSCVCTEPPNPSNETLAQWKKDNITAH
NISWRNLTVSECKKLRGVFLGSACGHHGP
SEQ ID NO: 7
EL3, mouse:
Unformatted residues are fully conserved, bold
strongly conserved, italicized weakly conserved,
and underlined without consensus between the mouse
and human sequence.
KLFHLGEIYAFNMHNNLDELTSYTCVCAEPSNPSNETLELWKRKNITAYS
VSWGNLTVSECKTFHGMFVGSACGPHGP
SEQ ID NO: 8
EL4, human: PSPKLHVPEKFEPTHPERGWIISPLGDNPW
SEQ ID NO: 9
EL4, mouse: PSPKLHVPEKFEPTDPSRGWIISPLGDNPW
SEQ ID NO: 10
EL5, human and mouse:
SISHVNSLKVESECSAPGEQPKFLGIREQR
SEQ ID NO: 11
human
MERFRLEKKLPGPDEEAVVDLGKTSSTVNTKFEKEELESHRAVYIGVHVP
FSKESRRRHRHRGHKHHHRRRKDKESDKEDGRESPSYDTPSQRVQFILGT
EDDDEEHIPHDLFTEMDELCYRDGEEYEWKETARWLKFEEDVEDGGDRWS
KPYVATLSLHSLFELRSCILNGTVMLDMRASTLDEIADMVLDNMIASGQL
DESIRENVREALLKRHHHQNEKRFTSRIPLVRSFADIGKKHSDPHLLERN
GEGLSASRHSLRTGLSASNLSLRGESPLSLLLGHLLPSSRAGTPAGSRCT
TPVPTPQNSPPSSPSISRLTSRSSQESQRQAPELLVSPASDDIPTVVIHP
PEEDLEAALKGEEQKNEENVDLTPGILASPQSAPGNLDNSKSGEIKGNGS
GGSRENSTVDFSKVDMNFMRKIPTGAEASNVLVGEVDFLERPIIAFVRLA
PAVLLTGLTEVPVPTRFLFLLLGPAGKAPQYHEIGRSIATLMTDEIFHDV
AYKAKDRNDLLSGIDEFLDQVTVLPPGEWDPSIRIEPPKSVPSQEKRKIP
VFHNGSTPTLGETPKEAAHHAGPELQRTGRLFGGLILDIKRKAPFFLSDF
KDALSLQC
SEQ ID NO: 12
human
ATVLSISHVNSLKVESECSAPGEQPKFLGIREQRVT
SEQ ID NO: 13
human
DRIKLFGMPAKHQPDLIYLRYVPLWKVHIFTVIQLTC
SEQ ID NO: 14
NBCn1_EL3h1.1:
[Hz]-HNNLDKLTSYSCVCTEPPNPSNETLAQWKKDNITA-amide
(SEQ ID NO: 14)
SEQ ID NO: 15
NBCn1_EL3h2.1:
[Hz]-LAQWKKDNITAHNISWRNLTVSECKKLRGVFLGSA-amide
(SEQ ID NO: 15).

1. An antibody or antigen binding fragment thereof specifically recognizing and binding an extracellular polypeptide region of human NBCn1.

2. The antibody or antigen binding fragment thereof according to claim 1, wherein said extracellular polypeptide region comprises or consists of a sequence selected from any one of SEQ ID NOS: 5, 6, 7, 8, 9, 10, 11, 12, 13, 14, and 15.

3. The antibody or antigen binding fragment thereof according to claim 1, wherein said extracellular polypeptide region comprises or consists of a sequence selected from SEQ ID NOS: 14 and 15.

4. The antibody or antigen binding fragment thereof according to claim 3, wherein said antibody or antigen binding fragment thereof is capable of inhibiting cellular NBCn1-mediated Na⁺-dependent HCO₃⁻ transport.

5. A method of selecting an antibody, said method comprising
a) providing an antibody library,
b) providing one or more antigens from an extracellular polypeptide region of human NBCn1,
c) contacting said antibody library with said one or more antigens, and
d) selecting antibodies, which bind said one or more antigens.

6. A method of producing an antibody specifically recognizing and binding an extracellular polypeptide region of human NBCn1, wherein said antibody is capable of inhibiting cellular NBCn1-mediated Na⁺-dependent HCO₃⁻-transport, said method comprising the steps of administering to an animal an extracellular polypeptide fragment of human NBCn1 or a homolog at least 98% identical thereto, isolating antibodies from said animal, testing whether antibodies are capable of inhibiting cellular NBCn1-mediated Na⁺-dependent HCO₃⁻ transport and selecting antibodies capable of inhibiting cellular NBCn1-mediated Na⁺-dependent HCO₃⁻ transport.

7. The method according to claim 6, wherein said extracellular polypeptide fragment is selected from the group consisting of SEQ ID NOS: 5, 6, 7, 8, 9, 10, 11, 12, 13, 14, and 15.

8. An antibody obtainable by a method of any one of claims 5 and 6.

9.-13. (canceled)

14. A method of treating breast cancer, atherosclerosis and/or restenosis comprising administering a pharmaceutically acceptable amount of an antibody as defined in claim 1 to a subject in need thereof.

15. A method of inhibiting cellular NBCn1-mediated Na⁺-dependent HCO₃⁻ transport, said method comprising providing a sufficient amount of an antibody as defined in claim 1 to target cells.

16. The method according to claim 14, wherein said breast cancer is a triple negative breast cancer, an estrogen receptor-positive breast cancer or a HER2-positive breast cancer.

17. The method according to claim 14, further comprising administering to said subject at least one additional agent.

18. The method according to claim 17, wherein said at least one additional agent is an additional anti breast cancer agent.

19. The method according to claim 18, wherein the additional anti breast cancer agent is an estrogen-receptor antibody or a HER2 antibody regulator of Na⁺,H⁺exchanger (NHE), a monocarboxylic acid transporter (MCT) or a proton pump inhibitor (PPI).
